(19) United States
(12) Patent Application Publication
(10) Pub. No.: US 2013/0101684 A1
(43) Pub. Date: Apr. 25, 2013

(54) Compounds, Compositions, And Methods For Reducing Or Eliminating Bitter Taste

(71) Applicants: Jane V. Leland, Wilmette, IL (US); Louise Slade, Morris Plains, NJ (US); David Hayashi, Chicago, IL (US); William P. Jones, Skokie, IL (US); Peter H. Brown, Glenview, IL (US); Joseph Gunnet, Flemington, NJ (US); Daniel Lavery, Princeton, NJ (US); Jessica Langer, Highland Park, NJ (US); Kambiz Shekdar, New York, NY (US)

(72) Inventors: Jane V. Leland, Wilmette, IL (US); Louise Slade, Morris Plains, NJ (US); David Hayashi, Chicago, IL (US); William P. Jones, Skokie, IL (US); Peter H. Brown, Glenview, IL (US); Joseph Gunnet, Flemington, NJ (US); Daniel Lavery, Princeton, NJ (US); Jessica Langer, Highland Park, NJ (US); Kambiz Shekdar, New York, NY (US)

(73) Assignee: Chromocell Corporation, North Brunswick, NJ (US)

(21) Appl. No.: 13/641,209

(22) PCT Filed: Apr. 15, 2011
(86) PCT No.: PCT/US11/32782

§ 371 Date: Dec. 21, 2012

Related U.S. Application Data
(60) Provisional application No. 61/324,362, filed on Apr. 15, 2010.
(60) Provisional application No. 61/324,359, filed on Apr. 15, 2010.
(60) Provisional application No. 61/324,351, filed on Apr. 15, 2010.
(60) Provisional application No. 61/324,346, filed on Apr. 15, 2010.
(60) Provisional application No. 61/324,345, filed on Apr. 15, 2010.

(51) Int. Cl. A23L 1/22; C07C 251/24; C07C 217/58; C07C 321/28; C07D 319/20; C07D 215/48; C07D 219/08; C07D 215/233; C07C 233/25; C07C 235/24; C07C 43/205; C07D 231/14

(52) U.S. Cl. CPC A23L 1/22083; C07C 43/2055; C07C 251/24; C07C 217/58; C07C 321/28; C07D 231/14; C07D 215/48; C07D 219/08; C07D 215/233; C07C 233/25; C07C 235/24; C07D 319/20
USPC 424/679; 568/644; 564/248; 514/788; 564/389; 548/374.1; 514/785; 546/79; 544/361; 546/104; 544/126; 544/363; 564/223; 564/175; 549/366; 514/784; 514/769; 514/777; 426/534; 426/535; 426/537

(57) ABSTRACT

The present invention provides edible compositions comprising a compound of the present invention, food products comprising such edible compositions and methods of preparing such food products. The present invention also provides methods of reducing the amount of NaCl in a food product, methods of reducing the sodium intake in a diet, and methods of reducing bitter taste in a food product.

### FIELD OF THE INVENTION

The present invention relates to flavor in edible compositions.

### BACKGROUND OF THE INVENTION

The sense of taste, e.g., in humans, can detect at least five traditional tastes: sweet, sour, salty, bitter, and umami (savory). Many nutritious substances including vegetables, foods, food ingredients and nutrients comprise bitter tastants and/or have a bitter taste. In addition, many pharmaceutical substances important to maintain or improve health comprise bitter tastants and/or have a bitter taste. While certain food products and consumer products have desirable bitter tastes, including coffee, beer and dark chocolate, in many contexts, consumers dislike such bitter tastes. For example, many consumers dislike the perception of certain bitter tastants and/or bitter taste and will avoid food or pharmaceutical products with an undesirable bitter tastant or bitter taste in favor of food or pharmaceutical products that have reduced levels of undesirable bitter tastants or that have reduced or that completely lack bitter taste. This aversion to products containing undesirable bitter tastants and/or having undesirable bitter taste may be caused by perception of bitter tastants and/or bitter taste mediated by activation of bitter receptors present in the oral cavity and/or in the gastrointestinal tract. In many cases, consumer dislike of bitter tastants and/or bitter taste prevents or hampers improvement of the nutritive quality and safety of foods as desired levels of nutrients or preservatives comprising bitter tastants and/or having bitter taste cannot be used. Also, dislike of or aversion to the bitter tastants or bitter taste of some pharmaceutical agents negatively impacts compliance with prescribed regimens for their use.

For instance, several additives, preservatives, emulsifiers and foodstuffs used in the production of food products comprise bitter tastants and/or have a bitter taste. While these additives, preservatives, emulsifiers and foodstuffs may affect the taste of a food product, they may also be important for improving the shelf life, nutritive quality, or texture of the food product. For example, the increasing trend of hypertension and cardiovascular disease has been attributed, in part, to the high sodium intake of the Western diet. Accordingly, substitution of sodium chloride with another salty tasting compound is desirable. The most common sodium chloride substitute is potassium chloride, which, to a portion of the population, is perceived as possessing a bitter taste in addition to its salty taste. The bitter taste of potassium chloride limits the extent to which it may be used to replace sodium chloride in foods without causing undesired bitter taste for the portion of the population sensitive to it.

Another common food additive, sodium lactate, has a broad antimicrobial action, is effective at inhibiting spoilage, and growth of pathogenic bacteria, and is commonly used in food products (e.g., meat and poultry products) to extend shelf life and increase food safety. Due to its sodium content, however, sodium lactate, can be undesirable as a preservative. Potassium lactate, which has similar antimicrobial properties, has been used in lieu of sodium lactate. However, potassium lactate is also associated with a bitter taste which limits the extent to which it may be used to replace sodium lactate in foods without causing undesired bitter taste.

In addition, the increasing incidence of obesity and diabetes has been attributed, in part, to the high sugar intake of many diets. Accordingly, substitution of sugar with another sweet tasting compound is desirable. Artificial and natural sugar substitutes that may be used to reduce sugar in foods are often associated with bitter taste which again limit the extent to which these may be used to replace sugar in foods without causing adverse bitter taste. For example, a common sugar substitute is Acesulfame K, which also has a bitter taste in addition to its sweet taste.

Without being limited by theory, bitter, sweet, and umami tastants and compounds typically elicit a taste response via G-protein coupled receptors, while salty and sour tastants and compounds are typically hypothesized to elicit a taste response via ion channels. Bitter taste receptors belong to the T2R (also referred to as TAS2R) family of G-protein coupled receptors that induce intracellular calcium concentration changes in response to a bitter tastant. T2R receptors act via gustducin, a taste-specific G-protein. There are at least twenty-five different members of the T2R family, suggesting that the perception of bitter taste is complex, involving several different tastant-receptor interactions. Compounds capable of modulating the activation and/or signaling of bitter taste receptors in the oral cavity and/or the gastrointestinal tract could be effective to allow desired usage levels of bitter tastants or bitter tasting substances in food and pharmaceutical products without resulting in consumer dislike of such products due to perception of the increased levels of bitter tastants or bitter tastes. In some instances, blockers or modulators of bitter taste receptors and bitter taste may reduce the perception of bitter tastants and/or bitter taste via the bitter taste receptors and/or taste transduction signaling machinery present in the oral cavity and/or the gastrointestinal tract.

Traditionally in food preparation and pharmaceuticals, bitter taste was masked using sweeteners and other tastants, including salt. In some cases, however, this is undesirable or insufficient because it can alter, mask, or interfere with other tastes/flavors/impressions (e.g., non bitter tastes or desired bitter tastes) in the food product. Additionally, this approach has rarely been able to completely mask the bitter taste present in such food products or pharmaceuticals. For that reason, compounds which reduce bitter taste instead of, or in addition to, masking agents are preferred.

It is, therefore, desirable to provide compounds that may be added to food products, consumer products and pharmaceuticals comprising bitter tastants or having a bitter taste to eliminate, modulate or reduce the perception of the bitter tastants or bitter taste, or to reduce the corresponding activation of the bitter receptors in the oral cavity and/or the gastrointestinal tract. Similarly, it is desirable to provide food products, consumer products, and pharmaceutical compositions comprising such compounds. It is also desirable to decrease the sodium intake of a subject using such compounds to eliminate, modulate or reduce the perception of bitter taste associated with salt substitutes. It is further desirable to decrease the sugar intake of a subject using such compounds to eliminate, modulate or reduce the perception of bitter taste associated with sugar substitutes.

### SUMMARY OF THE INVENTION

The present invention provides compounds that modulate bitter taste, edible compositions comprising such compounds, and methods of preparing such edible compositions. The present invention also provides methods of reducing the amount of sodium or sugar in an edible composition and methods of reducing bitter taste of a food product. The present invention further provides a method of reducing, modulating or eliminating the bitter taste of a food, consumer or pharmaceutical product in a subject. The present invention also provides a method of modulating, particularly reducing the activation of a bitter taste receptor.

### Edible Compositions

One aspect of the present invention provides edible compositions for reducing bitter taste of a bitter tastant. In some embodiments, the edible composition comprises a diphenyl-containing compound. In some embodiments, the diphenyl-containing compound is a compound having a molecular weight less than about 1000, 500, or 300 daltons. In certain embodiments, the diphenyl-containing compound is a compound of Formula (I), Formula (IIa), Formula (IIb), Formula (IIIb), Formula (IIIb′) or Formula (IIIb″) or Compounds 1-22 or a comestibly or biologically acceptable salt or derivative thereof or an enantiomer or diastereomer thereof.

In some embodiments, the edible composition comprises a pyrazole-containing compound. In some embodiments, the pyrazole-containing compound is a compound having a molecular weight less than about 1000, 500, or 300 daltons. In certain embodiments, the pyrazole-containing compound is a compound of Formula (IV), Formula (Va), Formula (Vb), Formula (VIa), Formula (VIb), Formula (VIa) or Formula (VIIb) or Compounds 23-36 or a comestibly or biologically acceptable salt or derivative thereof, or an enantiomer or diastereomer thereof.

In some embodiments, the edible composition comprises a hydroquinoline compound. In some embodiments, the hydroquinoline compound is a compound having a molecular weight less than about 1000, 500, or 300 daltons. In certain embodiments, the hydroquinoline compound is a compound of Formula (VIII), Formula (IX), or Formula (X) or Compounds 37-43 or a comestibly or biologically acceptable salt or derivative thereof, or an enantiomer or diastereomer thereof.

In some embodiments, the edible composition comprises a quinoline compound. In some embodiments, the quinoline compound is a compound having a molecular weight less than about 1000, 500, or 300 daltons. In certain embodiments, the quinoline compound is a compound of Formula (XI), Formula (XIIa), Formula (XIIb), Formula (XIIIa), or Formula (XIIIb) or Compounds 44-48 or a comestibly or biologically acceptable salt or derivative thereof, or an enantiomer or diastereomer thereof.

In some embodiments, the edible composition comprises a N-phenylalkylamide compound. In some embodiments, the N-phenylalkylamide compound is a compound having a molecular weight less than about 1000, 500, or 300 daltons. In certain embodiments, the N-phenylalkylamide compound is a compound of Formula (XIV), Formula (XVa), Formula (XVb), or Formula (XVc) or Compounds 49-58 or a comestibly or biologically acceptable salt or derivative thereof, or an enantiomer or diastereomer thereof.

In some embodiments, the edible composition comprises (a) a compound of the invention; and (b) a bitter tastant. In some embodiments, the compound of the invention is a compound having a molecular weight less than about 1000, 500, or 300 daltons. In certain embodiments, the compound of the invention is a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIa), Formula (XIIIb), Formula (XIV), Formula (XVa), Formula (XVb) or Formula (XVc), as described herein, or combinations thereof.

In another embodiment, the edible composition comprises (a) any one of Compounds 1-58, or combinations thereof; and (b) a bitter tastant.

According to the invention, the bitter tastant can be inherent in, e.g., a food product (such as coffee or chocolate) or can be a component of an edible composition (such as a bitter tasting preservative). In some embodiments, the bitter tastant present in the edible composition is a bitter tasting salt. In some embodiments, the bitter tastant present in the edible composition is a potassium salt, a magnesium salt, or a calcium salt. In some embodiments, the bitter tastant is a potassium salt. In some embodiments, the bitter tastant present in the edible compositions is KCl. In other embodiments, the bitter tastant present in the edible composition is potassium lactate.

In some embodiments, the edible composition further comprises a sodium salt. In some embodiments, the edible composition further comprises NaCl. In other embodiments, the edible composition further comprises sodium lactate. In some embodiments, the edible composition further comprises sugar.

In another aspect of the invention, the edible composition is a food product comprising at least one compound of the invention. In certain embodiments, the compound of the invention is a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof.

In another embodiments, the pharmaceutical composition comprises a bitter tasting pharmaceutically active ingredient and any one of Compounds 1-58, or combinations thereof. In another aspect of the present invention, the edible composition is a pharmaceutical composition comprising a bitter tasting pharmaceutically active ingredient and a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof. In another embodiments, the pharmaceutical composition comprises a bitter tasting pharmaceutically active ingredient and any one of Compounds 1-58, or combinations thereof.

In yet other embodiments, the edible composition is a pharmaceutical composition comprising a pharmaceutically active ingredient, a bitter tastant, and a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof. In yet other embodiments, the pharmaceutical composition comprises a pharmaceutically active ingredient, a bitter tastant, and any one of Compounds 1-58, as described herein, or combinations thereof.

In another aspect of the present invention, the edible composition is a consumer product comprising a bitter tastant and a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof. In another embodiments, the consumer product comprises a bitter tasting ingredient and any one of Compounds 1-58, or combinations thereof.

Yet another embodiment of the present invention provides a consumer product for reducing bitter taste of a bitter tastant, wherein said consumer product comprises a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof. In yet other embodiments, the consumer product for reducing bitter taste of a bitter tastant comprises any one of Compounds 1-58, as described herein, or combinations thereof.

In a further aspect, the present invention provides a method of preparing an edible composition comprising:
(a) providing a comestibly acceptable carrier; and
(b) adding to the comestibly acceptable carrier of (a) a compound of Formula (I), Formula (IIb), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof.

In another embodiment, the method of preparing an edible composition comprises:
(a) providing a comestibly acceptable carrier; and
(b) adding to the comestibly acceptable carrier of (a) any one of Compounds 1-58, or combinations thereof.

In some embodiments, the edible composition is a food product, a consumer product or a pharmaceutical composition. In some embodiments, the comestibly acceptable carrier is a foodstuff, a food product, or a pharmaceutically acceptable carrier.

In some embodiments, the comestibly acceptable carrier in (a) is inherently bitter. In such embodiments, the comestibly acceptable carrier may inherently contain a bitter tastant (i.e., the comestibly acceptable carrier is bitter without addition of a bitter tastant). In some embodiments, the inherent bitter tastant is a bitter tasting salt. In some embodiments, the inherently bitter comestibly acceptable carrier comprises a potassium salt, a magnesium salt, or a calcium salt. In some embodiments, the inherently bitter comestibly acceptable carrier comprises a potassium salt, such as KCl.

In other embodiments, the method of preparing an edible composition further comprises: (c) adding a bitter tastant. In some embodiments, the bitter tastant used in the methods of preparing an edible composition is a bitter tasting salt. In some embodiments, the bitter tastant used in the methods of preparing an edible composition is a potassium salt, a magnesium salt, or a calcium salt. In some embodiments, the bitter tastant used in the methods of preparing an edible composition is a potassium salt. In some embodiments, the bitter tastant used in the methods of preparing an edible composition is KCl. In other embodiments, the bitter tastant used in the methods of preparing an edible composition is potassium lactate.

In some embodiments, the edible composition further comprises a sodium salt. In some embodiments, the edible composition further comprises NaCl. In some embodiments, the edible composition further comprises sodium lactate. In some embodiments, the edible composition further comprises sugar.

The present invention also provides a method of reducing the amount of sodium in an edible composition. In some embodiments, such methods comprise:
(a) replacing an amount of one or more sodium salts used to prepare an edible composition with an amount of one or more potassium salts; and
(b) incorporating into the edible composition an effective amount of a compound according to Formula (I), Formula (IIa), Formula (IIb), Formula (IIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof.

In another embodiment, the method of reducing the amount of sodium in an edible composition comprises:
(a) replacing an amount of one or more sodium salts used to prepare an edible composition with an amount of one or more potassium salts; and
(b) incorporating into the edible composition an effective amount of any one of Compounds 1-58, or combinations thereof.

In some embodiments, the edible composition is a food product, a consumer product or a pharmaceutical composition.

In some embodiments of the present invention, the method of reducing the amount of sodium in an edible composition, comprises adding an amount of the compound in (b) sufficient to permit replacement of up to 25% of the sodium present in an edible composition with potassium. In other embodiments, the amount of the compound added in (b) is sufficient to permit replacement of up to 50% of the sodium present in an edible composition with potassium. In yet other embodiments, the amount of the compound added in (b) is sufficient to permit replacement of up to 75% of the sodium present in an edible composition with potassium. In other embodiments, the amount of the compound added in (b) is sufficient to permit replacement of up to 100% of the sodium present in an edible composition with potassium. In some embodiments, the edible composition maintains a salty flavor.

The present invention also provides a method of reducing the amount of NaCl in an edible composition. In some embodiments, such methods comprise:
(a) replacing an amount of NaCl used to prepare an edible composition with an amount of KCl; and
(b) incorporating into the edible composition an effective amount of a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIa), Formula (VIIb), Formula (VIII), Formula (IX), Formula (X), Formula (XI), Formula (XIIa), Formula (XIIb), Formula (XIIa), Formula (XIIIb), Formula (XIV), Formula (XVa), Formula (XVb) or Formula (XVc), as described herein, or combinations thereof.

In another embodiment, the method of reducing the amount of NaCl in an edible composition comprises:
(a) replacing an amount of NaCl used to prepare an edible composition with an amount of KCl; and
(b) incorporating into the edible composition an effective amount of any one of Compounds 1-58, or combinations thereof.

In some embodiments, the edible composition is a food product, a consumer product or a pharmaceutical composition.

In some embodiments of the present invention, the method of reducing the amount of sodium in an edible composition, comprises adding an amount of the compound in (b) sufficient to permit replacement of up to 25% of the NaCl present in an edible composition with KCl. In other embodiments, the amount of the compound added in (b) is sufficient to permit replacement of up to 50% of the NaCl present in an edible composition with KCl. In yet other embodiments, the amount of the compound added in (b) is sufficient to permit replacement of up to 75% of the NaCl present in an edible composition with KCl. In other embodiments, the amount of the compound added in (b) is sufficient to permit replacement of up to 100% of the NaCl present in an edible composition with KCl. In some embodiments, the edible composition maintains a salty flavor.

In another embodiment, the present invention provides a method of reducing the amount of sodium lactate in an edible composition comprises:
(a) replacing an amount of sodium lactate used to prepare an edible composition with an amount of potassium lactate; and
(b) incorporating into the edible composition an effective amount of a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof.

In another embodiment, the invention provides a method of reducing the amount of sodium lactate in an edible composition comprising:
(a) replacing an amount of sodium lactate used to prepare an edible composition with an amount of potassium lactate; and
(b) incorporating into the edible composition an effective amount of any one of Compounds 1-58, or combinations thereof.

In some embodiments, the edible composition is a food product, a consumer product or a pharmaceutical composition.

In some embodiments of the present invention, the method of reducing the amount of sodium lactate in an edible composition, comprises adding an amount of the compound in (b) sufficient to permit replacement of up to 25% of the sodium lactate present in an edible composition with potassium lactate. In other embodiments, the amount of the compound added in (b) is sufficient to permit replacement of up to 50% of the sodium lactate present in an edible composition with potassium lactate. In yet other embodiments, the amount of the compound added in (b) is sufficient to permit replacement of up to 75% of the sodium lactate present in an edible composition with potassium lactate. In other embodiments, the amount of the compound added in (b) is sufficient to permit replacement of up to 100% of the sodium lactate present in an edible composition with potassium lactate. In some embodiments, the edible composition has the same shelf life as an edible composition comprising sodium lactate.

In another embodiment, the invention provides a method of reducing the amount of sugar in an edible composition comprising:
(a) replacing an amount of sugar used to prepare an edible composition with an amount of Acesulfame K; and
(b) incorporating into the edible composition an effective amount of a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof.

In another embodiment, the invention provides a method of reducing the amount of sugar in an edible composition comprising:
(a) replacing an amount of sugar used to prepare an edible composition with an amount of Acesulfame K; and
(b) incorporating into the edible composition an effective amount of any one of Compounds 1-58, or combinations thereof.

In some embodiments, the edible composition is a food product, a consumer product or a pharmaceutical composition.

In some embodiments of the present invention, the method of reducing the amount of sugar in an edible composition, comprises adding an amount of the compound in (b) sufficient to permit replacement of up to 25% of the sugar present in an edible composition with Acesulfame K. In other embodiments, the amount of the compound added in (b) is sufficient to permit replacement of up to 50% of the sugar present in an edible composition with Acesulfame K. In yet other embodiments, the amount of the compound added in (b) is sufficient to permit replacement of up to 75% of the sugar present in an edible composition with Acesulfame K. In other embodiments, the amount of the compound added in (b) is sufficient to permit replacement of up to 100% of the sugar present in an edible composition with Acesulfame K. In some embodiments, the edible composition maintains a sweet flavor.

The present invention also provides a method of reducing the sodium intake of a subject. Such method comprises:
(a) replacing an amount of NaCl used to prepare an edible composition with an amount of KCl; and
(b) incorporating into the edible composition an effective amount of a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIb), Formula (XIV), Formula (XVa), Formula (XVb) or Formula (XVc), as described herein, or combinations thereof, thereby reducing the sodium intake of the subject.

In another embodiment, the method of reducing the sodium intake of a subject comprises:
(a) replacing an amount of NaCl used to prepare an edible composition with an amount of KCl; and
(b) incorporating into the edible composition an effective amount of any one of Compounds 1-58, or combinations thereof, thereby reducing the sodium intake of the subject.

In another embodiment, the method of reducing the sodium intake of a subject comprises:
(a) replacing an amount of sodium lactate used to prepare an edible composition with an amount of potassium lactate; and
(b) incorporating into the edible composition an effective amount of a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, thereby reducing the sodium intake of the subject.

In another embodiment, the method of reducing the sodium intake of a subject comprises:
(a) replacing an amount of sodium lactate used to prepare an edible composition with an amount of potassium lactate; and
(b) incorporating into the edible composition an effective amount of any one of Compounds 1-58, or combinations thereof, thereby reducing the sodium intake of the subject.

In some embodiments, the edible composition is a food product, a consumer product or a pharmaceutical composition.

In some embodiments of the present invention, the methods of reducing the sodium intake of a subject further comprise (c) identifying a subject in need thereof. In some embodiments, the methods of reducing the sodium intake of a subject comprise adding an amount of the compound in (b) sufficient to reduce sodium intake by up to 25% using potassium replacement. In other embodiments, the amount of compound added in (b) is sufficient to reduce sodium intake by up to 50% using potassium replacement. In yet other embodiments, the amount of compound added in (b) is sufficient to reduce sodium intake by up to 75% using potassium replacement. In other embodiments, the amount of compound added in (b) is sufficient to reduce sodium intake by up to 100% using potassium replacement.

The present invention also provides a method of reducing sugar intake of a subject comprising:
(a) replacing an amount of sugar used to prepare an edible composition with an amount of Acesulfame K; and
(b) incorporating into the edible composition an effective amount of a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, thereby reducing the sugar intake of the subject.

In another embodiment, the method of reducing the sugar intake of a subject comprises:
(a) replacing an amount of sugar used to prepare an edible composition with an amount of Acesulfame K; and
(b) incorporating into the edible composition an effective amount of any one of Compounds 1-58, or combinations thereof, thereby reducing the sugar intake in the diet or meal of the subject.

In some embodiments, the edible composition is a food product, a consumer product or a pharmaceutical composition.

In some embodiments of the present invention, the methods of reducing the sugar intake of a subject further comprises (c) identifying a subject in need thereof. In some embodiments, the methods of reducing the sugar intake of a subject comprise adding an amount of the compound in (b) sufficient to reduce sugar intake by up to 25% using Acesulfame K replacement. In other embodiments, the amount of compound added in (b) is sufficient to reduce sugar intake by up to 50% using Acesulfame K replacement. In yet other embodiments, the amount of compound added in (b) is sufficient to reduce sugar intake by up to 75% using Acesulfame K replacement. In other embodiments, the amount of compound added in (b) is sufficient to reduce sugar intake by up to 100% using Acesulfame K replacement.

The present invention also provides a method of reducing the bitter taste attributed to a bitter tastant in an edible composition comprising adding an effective amount of a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, to the edible composition such that any bitter taste induced by the bitter tastant is reduced. In other embodiments, the compound added to the edible composition is any one of Compounds 1-58, or combinations thereof.

The present invention further provides a method of reducing the bitter taste attributed to a bitter tastant in an edible composition comprising ingesting an effective amount of a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIa), Formula (XIIIb), Formula (XIV), Formula (XVa), Formula (XVb) or Formula (XVc), as described herein, or combinations thereof, before, along with, or after the edible composition such that any bitter taste induced by the bitter tastant is reduced. In other embodiments, the compound ingested with the edible composition is any one of Compounds 1-58, or combinations thereof.

In some embodiments, the method reduces the bitter taste induced by the bitter tastant by up to 25%. In some embodiments, the method reduces the bitter taste induced by the bitter tastant by up to 50%. In other embodiments, the bitter taste induced by the bitter tastant is reduced by up to 75%. In yet other embodiments, the bitter taste induced by the bitter tastant is reduced by up to 100%. In some embodiments, the bitter tastant present in the edible composition is a bitter tasting salt. In some embodiments, the bitter tastant present in the edible composition is a potassium salt, a magnesium salt, or a calcium salt. In some embodiments, the bitter tastant present in the edible compositions is KCl. In other embodiments, the bitter tastant present in the edible composition is potassium lactate.

In further aspect, the present invention provides a method of preserving an edible composition comprising:
(a) providing an edible composition; and
(b) adding to the edible composition of (a) potassium lactate and an effective amount of a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof.

In another embodiment, the method of preserving or extending the shelf life of an edible composition comprises:
(a) providing an edible composition; and
(b) adding to the edible composition of (a) potassium lactate and an effective amount of any one of Compounds 1-58, or combinations thereof.

The present invention also provides a method of reducing the amount of sodium in an edible composition while preserving the edible composition. In some embodiments, such method comprises:
(a) replacing an amount of sodium lactate used to prepare an edible composition with an amount of potassium lactate; and
(b) incorporating into the edible composition an effective amount of a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof.

The present invention also provides a method of reducing the amount of sodium in an edible composition while preserving the edible composition. In some embodiments, such method comprises:
(a) replacing an amount of sodium lactate used to prepare an edible composition with an amount of potassium lactate; and
(b) incorporating into the edible composition an effective amount of any one of Compounds 1-58, or combinations thereof.

In some embodiments, the edible composition is a food product. In some embodiments, the edible composition is a consumer product. In some embodiments, the edible composition is a pharmaceutical composition.

The present invention also provides a method of reducing or eliminating bitter taste in a subject utilizing an edible composition comprising a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof. In other embodiments, the composition that reduces or eliminates a bitter taste in a subject comprises any one of Compounds 1-58, or combinations thereof.

In some embodiments, the bitter taste is inherent. In some embodiments, the bitter taste is due to a bitter tasting salt. In some embodiments, the bitter taste is due to a potassium salt, a magnesium salt, or a calcium salt. In some embodiments, the bitter taste is due to KCl. In other embodiments, the bitter taste is due to potassium lactate.

The present invention also provides a method of inhibiting or reducing the activation and/or signaling of a bitter taste receptor, wherein the method comprises contacting a bitter taste receptor with a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof. In other embodiments, the method comprises contacting a bitter taste receptor with any one of Compounds 1-58, or combinations thereof. In some embodiments, the bitter taste receptor is in the mouth. In other embodiments, the bitter taste receptor is in the gastrointestinal tract, for example, in the stomach. In other embodiments, the bitter taste receptor is in an in vitro assay.

Particular embodiments of the invention are set forth in the following numbered paragraphs:

1. A composition comprising a compound according to Formula (I):

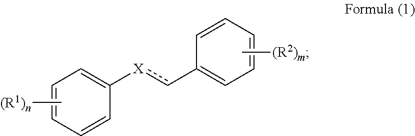

or a comestibly or biologically acceptable salt or derivative thereof, or an enantiomer or diastereomer thereof,
wherein, as valence and stability permit:
R¹, independently for each occurrence, is selected from the group consisting of C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, C₆₋₁₀aryloxy, C₆₋₁₀aryl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
R², independently for each occurrence, is selected from the group consisting of C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, C₆₋₁₀aryloxy, C₆₋₁₀aryl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
X is O or NR^{a}, wherein R^{a} is absent or is selected from the group consisting of hydrogen, C₁₋₁₀alkyl, C₁₋₁₀haloalkyl, C₂₋₁₀alkenyl, C₂₋₁₀alkynyl, carboxyl, C₁₋₁₀alkoxycarbonyl, C₂₋₁₀alkenyloxycarbonyl, C₂₋₁₀alkynyloxycarbonyl, C₁₋₁₀acyl, phosphoryl, phosphonate, phosphinate, cyano, sulfonate, sulfamoyl, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
wherein any of R¹, R², and R^{a}, independently and independently for each occurrence, is optionally substituted with 1-3 substituents selected from the group consisting of C₁₋₁₀alkyl, C₁₋₁₀haloalkyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl; and wherein heterocyclic or heteroaromatic rings, independently for each occurrence, comprise 1-4 heteroatoms selected from N, O, and S;
m is 1-3: and
n is 0-3;
wherein the composition is edible and capable of reducing bitter taste of a bitter tastant.

2. The composition according to paragraph 1, wherein as valence and stability permit:
R¹, independently for each occurrence, is selected from the group consisting of halo; hydroxyl; C₁₋₆alkyl; C₁₋₆haloalkyl, C₁₋₆hydroxylalkyl, or C₁₋₆acyloxy-C₁₋₆alkyl; C₂₋₆alkenyl; C₂₋₆alkynyl; C₁₋₆alkoxy; C₁₋₆alkylthio; and C₁₋₁₀aryl-C₁₋₆alkyloxy optionally substituted by halo, hydroxyl, C₁₋₆alkyl, C₁₋₆alkoxy, or C₁₋₆acyloxy;
R², independently for each occurrence, is selected from the group consisting of halo; hydroxyl; C₁₋₆alkyl; C₁₋₆haloalkyl, C₁₋₆hydroxylalkyl, or C₁₋₆acyloxy-C₁₋₆alkyl; C₂₋₆alkenyl; C₂₋₆alkynyl; C₁₋₆alkoxy; C₁₋₆alkylthio; and C₆₋₁₀aryl-C₁₋₆alkyloxy optionally substituted by halo, hydroxyl, C₁₋₆alkyl, C₁₋₆alkoxy, or C₁₋₆acyloxy;
X is O or NR^{a}, wherein R^{a} is absent or is selected from the group consisting of hydrogen and C₁₋₆alkyl;
wherein any of R¹, R², and R^{a}, independently and independently for each occurrence, is optionally further substituted as in paragraph 1;
m is 1-3; and
n is 0-3.

3. The composition according to paragraph 1, wherein said compound according to Formula (I) is a compound according to Formula (IIa):

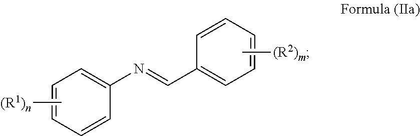

or a comestibly or biologically acceptable salt or derivative thereof, or an enantiomer or diastereomer thereof, wherein, as valence and stability permit, R¹, R², m, and n are as defined in paragraph 1.

4. The composition according to paragraph 1, wherein said compound according to Formula (I) is a compound according to Formula (IIIb):

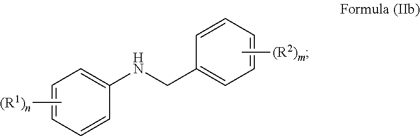

or a comestibly or biologically acceptable salt or derivative thereof, or an enantiomer or diastereomer thereof, wherein, as valence and stability permit, R¹, R², m, and n are as defined in paragraph 1.

5. The composition according to paragraph 4, wherein said compound according to Formula (IIb) is a compound according to Formula (IIIb):

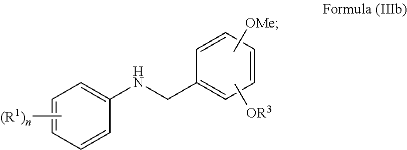

or a comestibly or biologically acceptable salt or derivative thereof, or an enantiomer or diastereomer thereof,
wherein, as valence and stability permit:
R¹ and n are as defined in paragraph 1; and
R³ is selected from the group consisting of methyl and ethyl.

6. The composition according to paragraph 4, wherein said compound according to Formula (IIb) is a compound according to Formula (IIIb′):

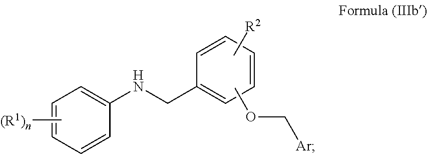

or a comestibly or biologically acceptable salt or derivative thereof, or an enantiomer or diastereomer thereof,
wherein, as valence and stability permit:
R¹, R², and n are as defined in paragraph 1; and
Ar is C₆₋₁₀aryl optionally substituted by halo, hydroxyl, C₁₋₆alkyl, C₁₋₆alkoxy, or C₁₋₆acyloxy.

7. The composition according to paragraph 4, wherein said compound according to Formula (IIb) is a compound according to Formula (IIIb″):

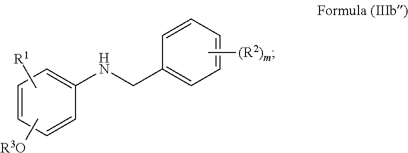

or a comestibly or biologically acceptable salt or derivative thereof, or an enantiomer or diastereomer thereof,
wherein, as valence and stability permit:
R¹, R², and m are as defined in paragraph 1; and
R³ is C₁₋₆alkyl, such as methyl.

8. The composition according to paragraph 1, wherein said compound according to Formula (I) is selected from the group consisting of:

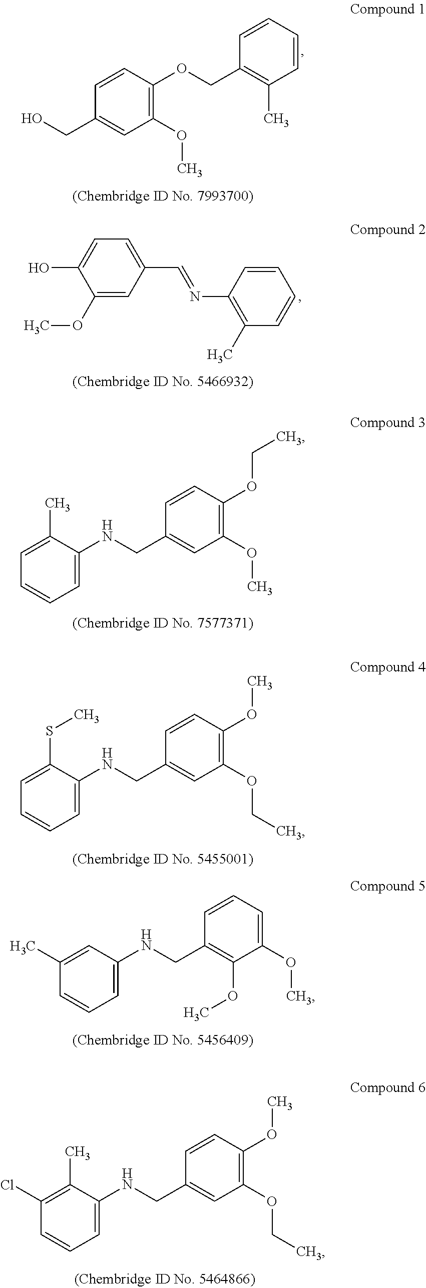 [SMILES: COc1cc(CO)ccc1OCc1ccccc1C ; COc1cc(/C=N/c2ccccc2C)ccc1O ; CCOc1ccc(CNc2ccccc2C)cc1OC ; CCOc1cc(CNc2ccccc2SC)ccc1OC ; COc1cccc(CNc2cccc(C)c2)c1OC ; CCOc1cc(CNc2cccc(Cl)c2C)ccc1OC]

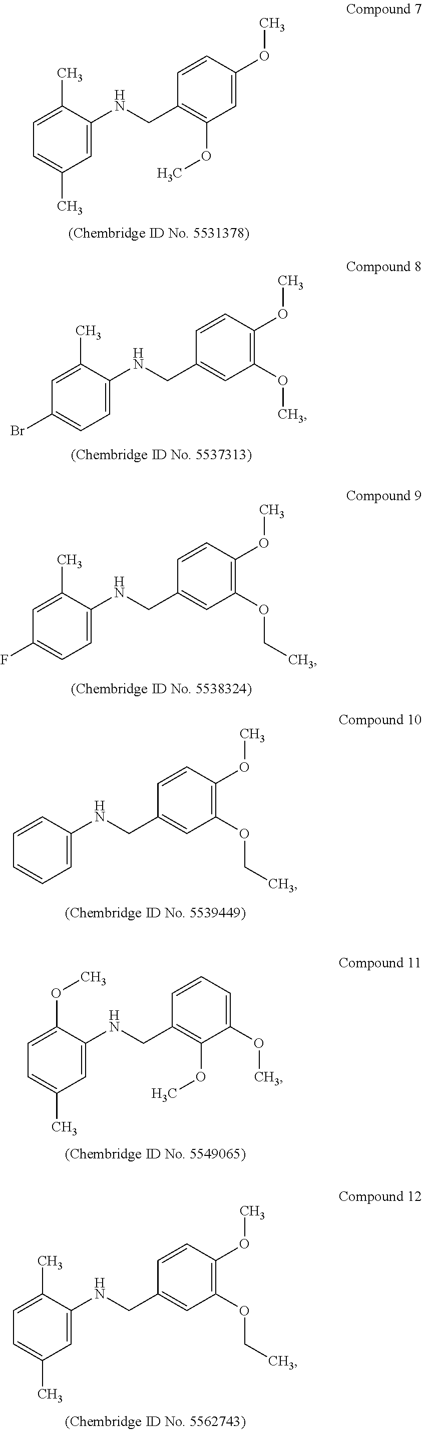 [SMILES: COc1ccc(CNc2cc(C)ccc2C)c(OC)c1 ; COc1ccc(CNc2ccc(Br)cc2C)cc1OC ; CCOc1cc(CNc2ccc(F)cc2C)ccc1OC ; CCOc1cc(CNc2ccccc2)ccc1OC ; COc1ccc(C)cc1NCc1cccc(OC)c1OC ; CCOc1cc(CNc2cc(C)ccc2C)ccc1OC]

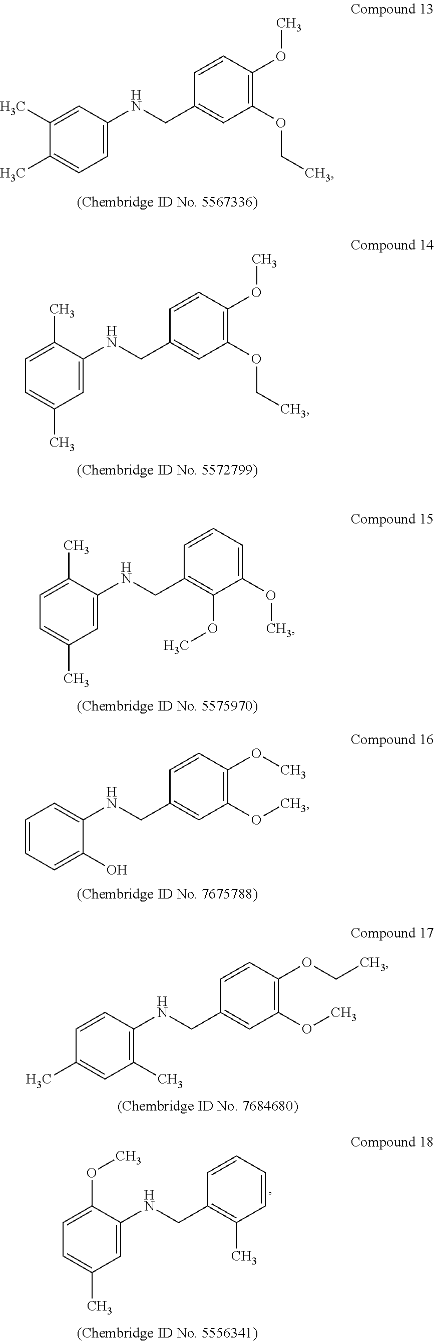

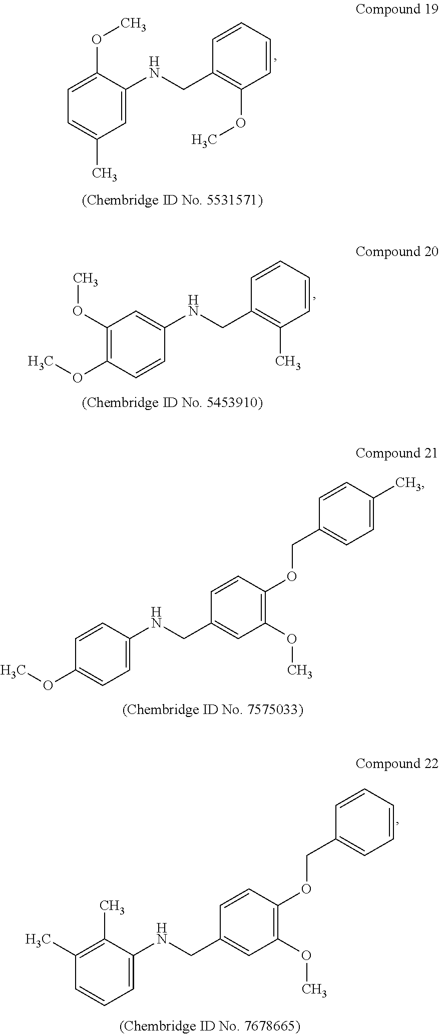 [SMILES: COc1ccccc1CNc1cc(C)ccc1OC ; COc1ccc(NCc2ccccc2C)cc1OC ; COc1ccc(NCc2ccc(OCc3ccc(C)cc3)c(OC)c2)cc1 ; COc1cc(CNc2cccc(C)c2C)ccc1OCc1ccccc1]

comestibly or biologically acceptable derivatives thereof, or an enantiomer or diastereomer thereof.

9. A composition comprising a compound according to Formula (IV):

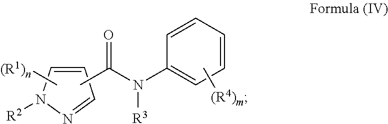

or a comestibly or biologically acceptable salt or derivative thereof, or an enantiomer or diastereomer thereof,
wherein, as valence and stability permit:
R¹, independently for each occurrence, is selected from the group consisting of C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkenyloxycarbonyl, C₁₋₁₀acyl, C₁₋₁₀acylamino, C₁₋₁₀acyloxy, C₁₋₁₀carbonate, C₁₋₁₀alkoxy, phenyloxy, phenyl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₅alkyl, wherein heterocyclic or heteroaromatic rings, independently for each occurrence, comprise 1-4 heteroatoms selected from N, O, and S;
R² is selected from the group consisting of hydrogen, C₁₋₁₀alkyl, C₁₋₁₀haloalkyl, C₂₋₁₀alkenyl, C₂₋₁₀alkynyl, carboxyl, C₁₋₁₀alkoxycarbonyl, C₂₋₁₀alkenyloxycarbonyl, C₂₋₁₀alkenyloxycarbonyl, C₁₋₁₀acyl, phosphoryl, phosphonate, phosphinate, cyano, sulfonate, sulfamoyl, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
R³ is selected from the group consisting of hydrogen, C₁₋₁₀alkyl, C₁₋₁₀haloalkyl, C₂₋₁₀alkenyl, C₂₋₁₀alkynyl, carboxyl, C₁₋₁₀alkoxycarbonyl, C₂₋₁₀alkenyloxycarbonyl, C₂₋₁₀alkenyloxycarbonyl, C₁₋₁₀acyl, phosphoryl, phosphonate, phosphinate, cyano, sulfonate, sulfamoyl, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
R⁴, independently for each occurrence, is selected from the group consisting of C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkenyloxycarbonyl, C₁₋₁₀acyl, C₁₋₁₀acylamino, C₁₋₁₀acyloxy, C₁₋₁₀carbonate, C₁₋₁₀alkoxy, phenyloxy, phenyl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
wherein any of R¹, R², R³, and R⁴, independently and independently for each occurrence, is optionally substituted with 1-3 substituents selected from the group consisting of C₁₋₁₀alkyl, C₁₋₁₀haloalkyl, halo, hydroxyl, carboxyl, C₁₋alkoxycarbonyl, C₂₋₁₀alkenyloxycarbonyl, C₂₋₁₀alkenyloxycarbonyl, C₁₋₁₀acyl, C₁₋₁₀acylamino, C₁₋₁₀acyloxy, C₁₋₁₀carbonate, C₁₋₁₀alkoxy, phenyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl; and wherein heterocyclic or heteroaromatic rings, independently for each occurrence, comprise 1-4 heteroatoms selected from N, O, and S;
n is 0-2: and
m is 0-3;
wherein the composition is edible and capable of reducing bitter taste of a bitter tastant.

10. The composition according to paragraph 9, wherein:
R¹, independently for each occurrence, is selected from the group consisting of halo, C₁₋₆alkyl, C₂₋₆alkenyl, and C₂₋₆alkynyl;
R² is selected from the group consisting of hydrogen, C₁₋₆alkyl, C₂₋₆alkenyl, C₂₋₆alkynyl, and C₁₋₆acyl;
R³ is selected from the group consisting of hydrogen, C₁₋₆alkyl, C₂₋₆alkenyl, and C₂₋₆alkynyl;
R⁴, independently for each occurrence, is selected from the group consisting of halo, C₁₋₆alkyl, C₂₋₆alkenyl, C₂₋₆alkynyl, C₁₋₆alkoxy, —C(O)—R⁵, and —C(O)—.N(R⁵)₂;
R⁵, independently for each occurrence, is selected from the group consisting of hydrogen, C₁₋₆alkyl, C₁₋₆alkenyl and C₂₋₆alkynyl;
wherein any of R¹, R², R³, and R⁴, independently and independently for each occurrence is optionally substituted as noted paragraph 9;
n is 0-2; and
m is 0-3.

11. The composition according to paragraph 9, wherein said compound according to Formula (IV) is a compound according to Formula (Va):

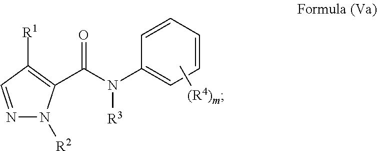

or a comestibly or biologically acceptable salt or derivative thereof, or an enantiomer or diastereomer thereof,
wherein, as valence and stability permit, R¹, R², R³, R⁴, and m are as defined in paragraph 9.

12, The composition according to paragraph 11, wherein said compound according to Formula (Va) is a compound according to Formula (VIa):

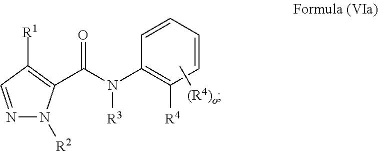

or a comestibly or biologically acceptable salt or derivative thereof, or an enantiomer or diastereomer thereof,
wherein, as valence and stability permit:
R¹, R², R³, and R⁴ are as defined in paragraph 11; and
o is 0-2.

13, The composition according to paragraph 12, wherein said compound according to Formula (VIa) is a compound according to Formula (VIIa):

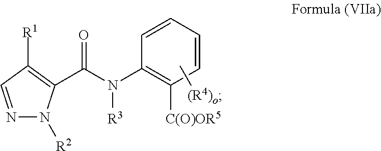

or a comestibly or biologically acceptable salt or derivative thereof, or an enantiomer or diastereomer thereof;
wherein, as valence and stability permit, R¹, R², R³, R⁴, R⁵, and o are as defined in paragraph 12.

14, The composition according to paragraph 9, wherein said compound according to Formula (IV) is a compound according to Formula (Vb):

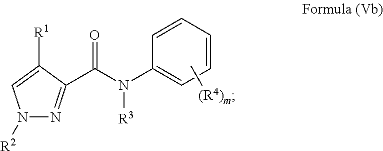

or a comestibly or biologically acceptable salt or derivative thereof, or an enantiomer or diastereomer thereof,
wherein, as valence and stability permit, R¹, R², R³, R⁴, and m are as defined in paragraph 9.

15. The composition according to paragraph 14 wherein said compound according to Formula (Vb) is a compound according to Formula (VIb):

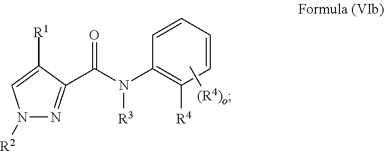

or a comestibly or biologically acceptable salt or derivative thereof, or an enantiomer or diastereomer thereof,
wherein, as valence and stability permit:
R¹, R², R³, R⁴, are as defined in paragraph 14; and
o is 0-2.

16. The composition according to paragraph 15 wherein said compound according to Formula (VIb) is a compound according to Formula (VIIb):

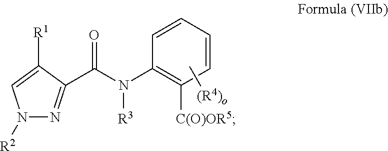

and comestibly or biologically acceptable derivatives thereof, or an enantiomer or diastereomer thereof,
wherein, as valence and stability permit, R¹, R², R³, R⁴, R⁵, and o are as defined in paragraph 9.

17. The composition according to paragraph 9, wherein said compound according to Formula (IV) is selected from the group consisting of:

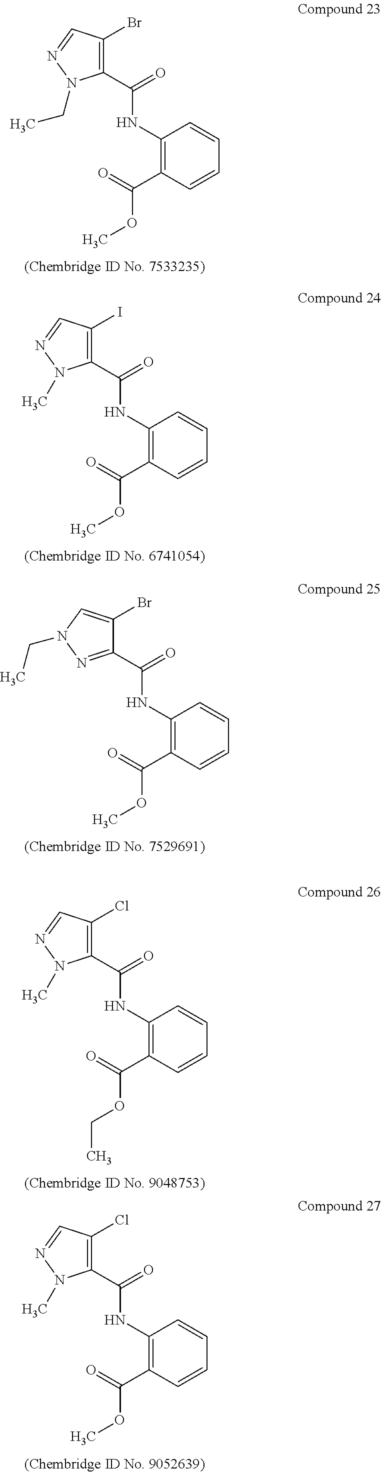 [SMILES: CCn1ncc(Br)c1C(=O)Nc1ccccc1C(=O)OC ; COC(=O)c1ccccc1NC(=O)c1c(I)cnn1C ; CCn1cc(Br)c(C(=O)Nc2ccccc2C(=O)OC)n1 ; CCOC(=O)c1ccccc1NC(=O)c1c(Cl)cnn1C ; COC(=O)c1ccccc1NC(=O)c1c(Cl)cnn1C]

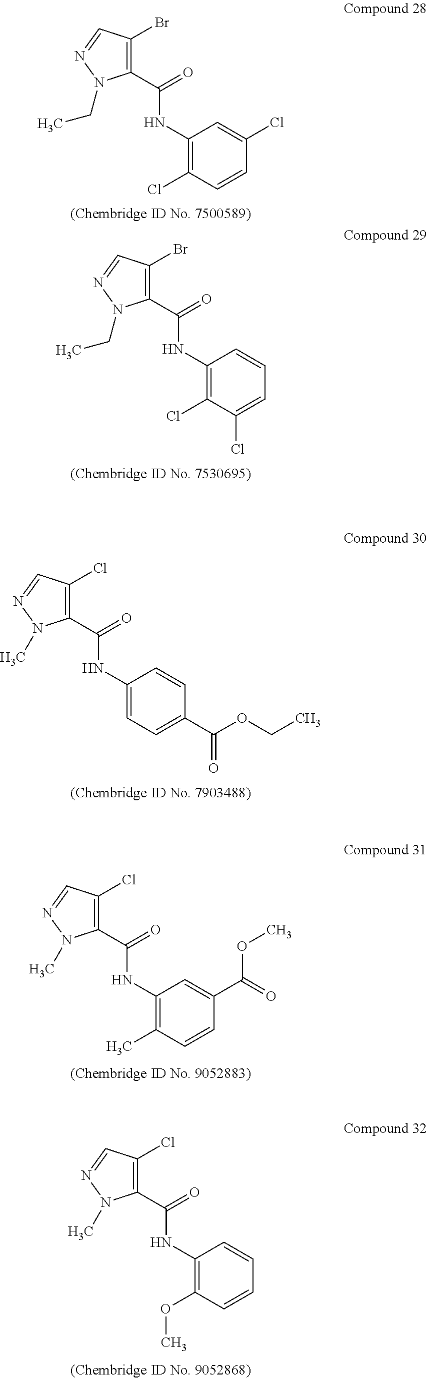 [SMILES: CCn1ncc(Br)c1C(=O)Nc1cc(Cl)ccc1Cl ; CCn1ncc(Br)c1C(=O)Nc1cccc(Cl)c1Cl ; CCOC(=O)c1ccc(NC(=O)c2c(Cl)cnn2C)cc1 ; COC(=O)c1ccc(C)c(NC(=O)c2c(Cl)cnn2C)c1 ; COc1ccccc1NC(=O)c1c(Cl)cnn1C]

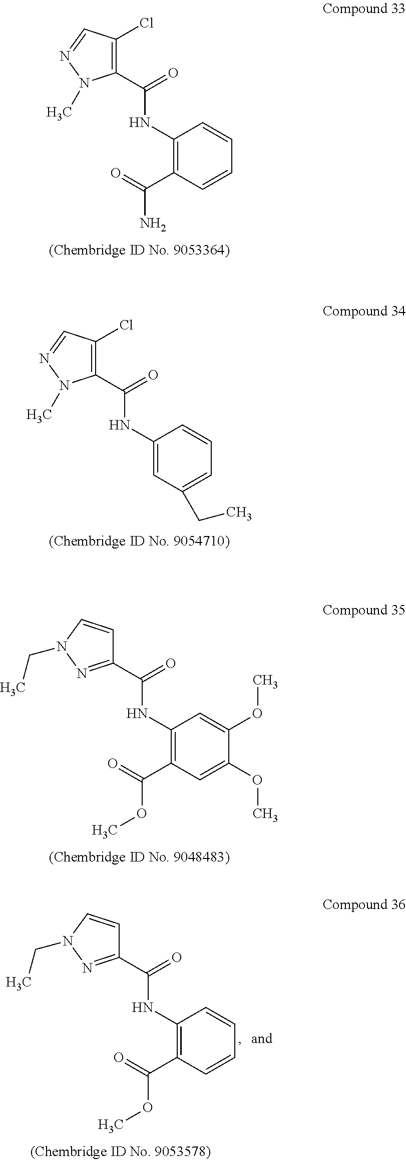

comestibly or biologically acceptable derivatives thereof, or an enantiomer or diastereomer thereof.

18, A composition comprising a compound according to Formula (VIII):

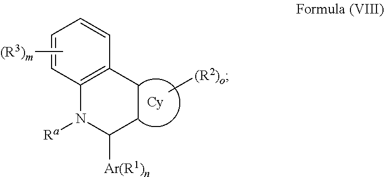

or a comestibly or biologically acceptable salt or derivative thereof, or an enantiomer or diastereomer thereof,
wherein, as valence and stability permit:
R¹, independently for each occurrence, is selected from the group consisting of C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkenyloxycarbonyl, C₁₋₁₀acyl, C₁₋₁₀acylamino, C₁₋₁₀acyloxy, C₁₋₁₀carbonate, C₁₋₁₀alkoxy, phenyloxy, phenyl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
R², independently for each occurrence, is selected from the group consisting of is selected from the group consisting of C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkenyloxycarbonyl, C₁₋₁₀acyl, C₁₋₁₀acylamino, C₁₋₁₀acyloxy, C₁₋₁₀carbonate, C₁₋₁₀alkoxy, phenyloxy, phenyl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₆heteroaryl-C₁₋₆alkyl, wherein heterocyclic or heteroaromatic rings, independently for each occurrence, comprise 1-4 heteroatoms selected from N, O, and S;
R³, independently for each occurrence, is selected from the group consisting of C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkenyloxycarbonyl, C₁₋₁₀acyl, C₁₋₁₀acylamino, C₁₋₁₀acyloxy, C₁₋₁₀carbonate, C₁₋₁₀alkoxy, phenyloxy, phenyl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₆heteroaryl-C₁₋₆alkyl, wherein heterocyclic or heteroaromatic rings, independently for each occurrence, comprise 1-4 heteroatoms selected from N, O, and S;
R^{a} is selected from the group consisting of hydrogen C₁₋₁₀alkyl, C₁₋₁₀haloalkyl, C₂₋₁₀alkenyl, C₂₋₁₀alkynyl, carboxyl, C₁₋₁₀alkoxycarbonyl, C₂₋₁₀alkenyloxycarbonyl, C₂₋₁₀alkenyloxycarbonyl, C₁₋₁₀acyl, phosphoryl, phosphonate, phosphinate, cyano, sulfonate, sulfamoyl, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
Ar is selected from the group consisting of C₆₋₁₀aryl and C₃₋₉heteroaryl;
Cy is a 5 to 7-membered carbocyclic or heterocyclic ring, wherein heterocyclic ring comprises 1-4 heteroatoms selected from N, O, and S;
wherein any of R¹, R², R³, and R^{a}, independently and independently for each occurrence, is optionally substituted with 1-3 substituents selected from the group consisting of C₁₋₁₀alkyl, C₁₋₁₀haloalkyl, halo, hydroxyl, carboxyl, C₁₋₁₀alkoxycarbonyl, C₂₋₁₀alkenyloxycarbonyl, C₂₋₁₀alkenyloxycarbonyl, C₁₋₁₀acyl, C₁₋₁₀acylamino, C₁₋₁₀acyloxy, C₁₋₁₀carbonate, C₁₋₁₀alkoxy, phenyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl; and wherein heterocyclic or heteroaromatic rings, independently for each occurrence, comprise 1-4 heteroatoms selected from N, O, and S;
m is 1-3;
n is 0-3; and
o is 0-3;
wherein the composition is edible and capable of reducing bitter taste of a bitter tastant.

19, The composition according to paragraph 18, wherein:
R¹, independently for each occurrence, is selected from the group consisting of halo, hydroxyl, C₁₋₆alkyl, C₁₋₆haloalkyl, C₂₋₆alkenyl, C₂₋₆alkynyl, C₁₋₆alkoxy, and C₁₋₆acyloxy;
R², independently for each occurrence, is C₁₋₆alkyl;
R³, independently for each occurrence, is selected from the group consisting of halo, C₁₋₆alkyl, C₂₋₆alkenyl, C₂₋₆alkenyl, C(O)—O—R⁴, and C(O)—N(R⁴)₂;
R⁴, independently for each occurrence, is selected from the group consisting of hydrogen, C₁₋₆alkyl, C₂₋₆alkenyl, and C₂₋₆alkynyl;
R^{a} is selected from the group consisting of hydrogen, C₁₋₆alkyl, C₂₋₆alkenyl, and C₂₋₆alkynyl;

Ar is selected from the group consisting of C₆₋₁₀aryl and C₃₋₉heteroaryl;
Cy is a 5 to 7-membered carbocyclic or heterocyclic ring, optionally including one or two carbon-carbon or carbon-nitrogen double bonds in the ring;
wherein any of R¹, R², R³, and R^{a}, independently and independently for each occurrence, is optionally substituted as described in paragraph 18;
m is 1-3;
n is 0-3: and
o is 0-3.

20. The composition according to paragraph 18, wherein said compound according to Formula (VIII) is a compound according to Formula (IX):

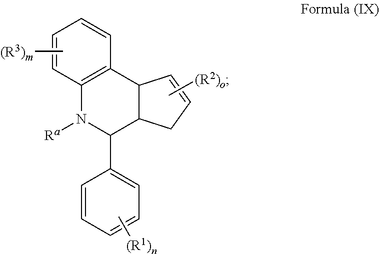

or a comestibly or biologically acceptable salt or derivative thereof or an enantiomer or diastereomer thereof, wherein, as valence and stability permit, R¹, R², R³, R^{a}, m, n, and o are as defined in paragraph 18.

21. The composition according to paragraph 20, wherein said compound according to Formula (IX) is a compound according to Formula (X):

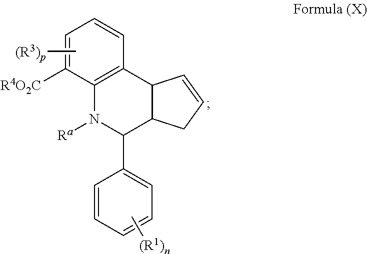

or a comestibly or biologically acceptable salt or derivative thereof, or an enantiomer or diastereomer thereof,
wherein, as valence and stability permit:
R¹, R³, R⁴, R^{a}, and n are as defined in paragraph 20; and
p is 0-2.

22. The composition according to paragraph 18, wherein said compound according to Formula (VIII) is selected from the group consisting of:

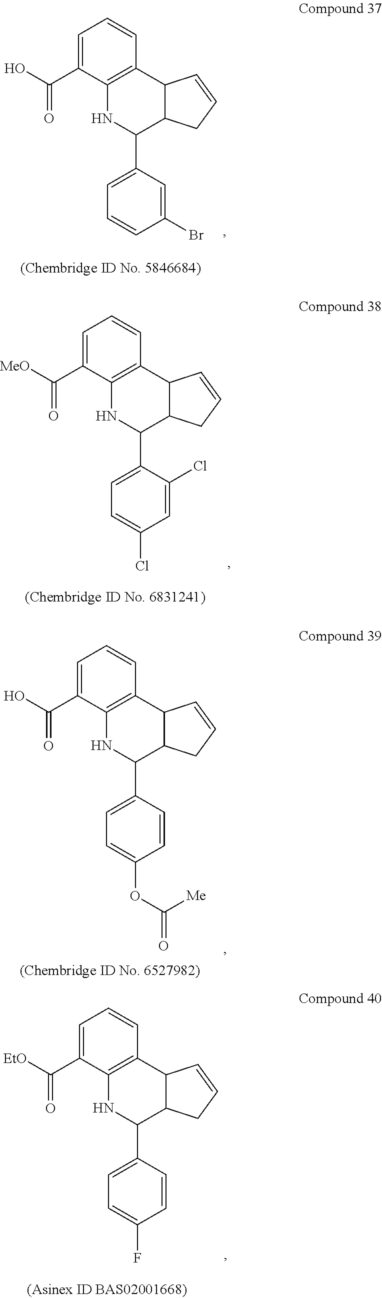 [SMILES: O=C(O)c1cccc2c1NC(c1cccc(Br)c1)C1CC=CC21 ; COC(=O)c1cccc2c1NC(c1ccc(Cl)cc1Cl)C1CC=CC21 ; CC(=O)Oc1ccc(C2Nc3c(C(=O)O)cccc3C3C=CCC32)cc1 ; CCOC(=O)c1cccc2c1NC(c1ccc(F)cc1)C1CC=CC21]

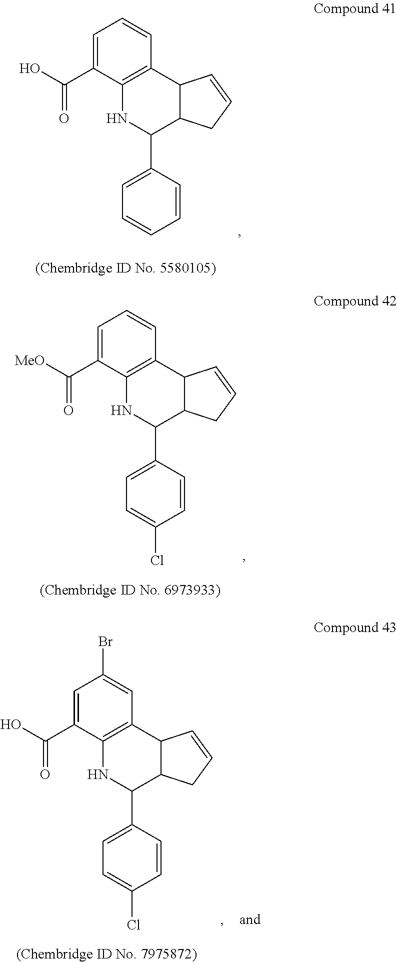 [SMILES: O=C(O)c1cccc2c1NC(c1ccccc1)C1CC=CC21 ; COC(=O)c1cccc2c1NC(c1ccc(Cl)cc1)C1CC=CC21 ; O=C(O)c1cc(Br)cc2c1NC(c1ccc(Cl)cc1)C1CC=CC21]

comestibly or biologically acceptable derivatives thereof.

23, A composition comprising a compound according to Formula (XI):

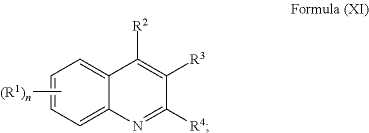

or a comestibly or biologically acceptable salt or derivative thereof, or an enantiomer or diastereomer thereof.
   wherein, as valence and stability permit:
   R¹, independently for each occurrence, is selected from the group consisting of C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkenyloxycarbonyl, C₁₋₁₀acyl, C₁₋₁₀acylamino, C₁₋₁₀acyloxy, C₁₋₁₀carbonate, C₁₋₁₀alkoxy, phenyloxy, phenyl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
   R² is selected from the group consisting of hydrogen, C₁₋₁₀alkyl, Het-C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkenyloxycarbonyl, C₁₋₁₀acyl, C₁₋₁₀acylamino, C₁₋₁₀acyloxy, C₁₋₁₀carbonate, C₁₋₁₀alkoxy, phenyloxy, phenyl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
   R³ is selected from the group consisting of hydrogen, C₁₋₁₀alkyl, Het-C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkenyloxycarbonyl, C₁₋₁₀acyl, C₁₋₁₀acylamino, C₁₋₁₀acyloxy, C₁₋₁₀carbonate, C₁₋₁₀alkoxy, phenyloxy, phenyl-C₁₋₆alkyloxy, C₁₋₅heteroaryloxy, C₁₋₅heteroaryl-C₁₋₆allyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
   R⁴ is selected from the group consisting of hydrogen, C₁₋₁₀alkyl, Het-C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkyloxycarbonyl, C₁₋₁₀acyl, C₁₋₁₀acylamino, C₁₋₁₀acyloxy, C₁₋₁₀carbonate, C₁₋₁₀alkoxy, phenyloxy, phenyl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
   or R³ and R⁴ together with the atoms to which they are attached form a 5 to 6-membered aryl or heteroaryl ring optionally substituted by 1 to 4 groups selected from the group consisting of Het, C₁₋₁₀alkyl, C₁₋₁₀haloalkyl, halo, hydroxy, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl; and wherein heterocyclic or heteroaromatic rings, independently for each occurrence, comprise 1-4 heteroatoms selected from N, O, and S;
   Het is a C₁₋₉heterocyclyl including 1-4 heteroatoms in the ring selected from oxygen, sulfur, and nitrogen;
   wherein any of R¹, R², R³, R⁴, and Het, independently and independently for each occurrence, is optionally substituted with 1-3 substituents selected from the group consisting of C₁₋₁₀alkyl, C₁₋₁₀haloalkyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀-acyloxy, C₁₋₁₀carbonate, C₁₋₁₀alkoxy, phenyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, C₆₋₁₀aryl, C₁₋₆alkyl-C₆₋₁₀aryl, C₆₋₁₀aryl-C₁₋₆alkyl, C₁₋₅heteroaryl, and C₁₋₅heteroaryl-C₁₋₆alkyl; and wherein heterocyclic or heteroaromatic rings, independently for each occurrence, comprise 1-4 heteroatoms selected from N, O, and S; and
   In is 0-4;
   wherein the composition is edible and capable of reducing bitter taste of a bitter tastant.

24. The composition according to paragraph 23, wherein as valence and stability permit:
R¹, independently for each occurrence, is selected from the group consisting of halo, hydroxyl, C₁₋₆alkyl, C₁₋₆haloalkyl, C₂₋₆alkenyl, C₂₋₆alkynyl, and C₁₋₆alkoxy;
R² is selected from the group consisting of hydrogen, halo, hydroxyl, C₁₋₆alkyl, C₁₋₆haloalkyl, Het-C₁₋₆alkyl, C₂₋₆alkenyl, C₂₋₆alkynyl, and C₁₋₆alkoxy;
R³ is selected from the group consisting of hydrogen, halo, hydroxyl, C₁₋₆alkyl, C₁₋₆haloalkyl, Het-C₁₋₆alkyl, C₂₋₆alkenyl, C₂₋₆alkynyl, and C₁₋₆alkoxy;
R⁴ is selected from the group consisting of hydrogen, halo, hydroxyl, C₁₋₆alkyl, C₁₋₆haloalkyl, Het-C₁₋₆alkyl, C₂₋₆alkenyl, C₂₋₆alkynyl, and C₁₋₆alkoxy;
or R³ and R⁴ together with the atoms to which they are attached form a 5 to 6-membered aryl ring optionally substituted by 1 to 4 groups selected from the group consisting of halo, hydroxyl, C₁₋₆alkyl, C₁₋₆haloalkyl, C₂₋₆alkenyl, C₂₋₆alkynyl, C₁₋₆alkoxy, and Het;
Het is a C₂₋₆heterocyclyl including 1-3 heteroatoms in the ring selected from oxygen, sulfur, and nitrogen and is optionally substituted with one or more groups selected from the group consisting of halo, hydroxyl, C₁₋₆alkyl, C₁₋₆haloalkyl, C₂₋₆alkenyl, C₂₋₆alkynyl, C₁₋₆alkoxy, and C₆₋₁₀aryl optionally substituted by C₁₋₆alkyl;
wherein any of R¹, R², R³, R⁴, and Het, independently and independently for each occurrence, is optionally further substituted as in paragraph 23; and

n is 0-4.

25, The composition according to paragraph 23, wherein said compound according to Formula (XI) is a compound according to Formula (XIIa):

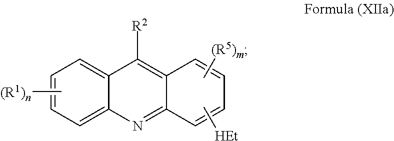

or a comestibly or biologically acceptable salt or derivative thereof, or an enantiomer or diastereomer thereof,
wherein, as valence and stability permit:
R¹, R², Het, and n are as defined in paragraph 23;
R⁵, independently for each occurrence, is selected from the group consisting of halo, hydroxyl, C₁₋₆alkyl, C₁₋₆haloalkyl, C₂₋₆alkenyl, C₂₋₆alkynyl, and C₁₋₆alkoxy; and
m is 0-3.

26. The composition according to paragraph 25, wherein said compound according to Formula (XIIa) is a compound according to Formula (XIIIa):

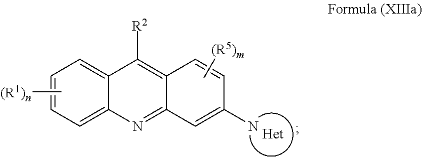

or a comestibly or biologically acceptable salt or derivative thereof, or an enantiomer or diastereomer thereof wherein, as valence and stability permit, R¹, R², R⁵, Het, n, and m are as defined in paragraph 25.

27. The composition according to paragraph 23, wherein said compound according to Formula (XI) is a compound according to Formula (XIIb):

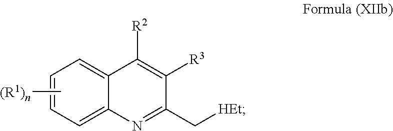

or a comestibly or biologically acceptable salt or derivative thereof, or an enantiomer or diastereomer thereof, wherein, as valence and stability permit, R¹, R², R³, Het, and n are as defined in paragraph 23.

28. The composition according to paragraph 27, wherein said compound according to Formula (XIIb) is a compound according to Formula (XIIIb):

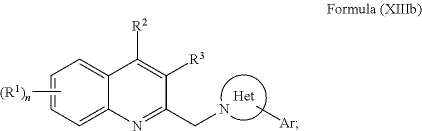

or a comestibly or biologically acceptable salt or derivative thereof, or an enantiomer or diastereomer thereof,
wherein, as valence and stability permit:
R¹, R², R³, Het, and n are as in paragraph 27; and
Ar is C₆₋₁₀aryl, such as phenyl or naphthyl, optionally substituted by C₁₋₆alkyl.

29. The composition according to paragraph 23, wherein said compound according to Formula (XI) is selected from the group consisting of:

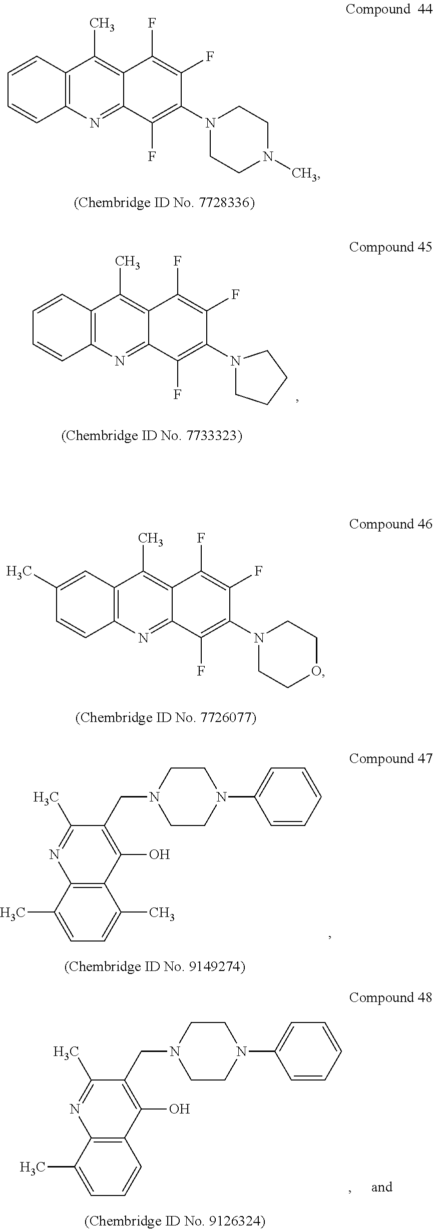 [SMILES: Cc1c2ccccc2nc2c(F)c(N3CCN(C)CC3)c(F)c(F)c12 ; Cc1c2ccccc2nc2c(F)c(N3CCCC3)c(F)c(F)c12 ; Cc1ccc2nc3c(F)c(N4CCOCC4)c(F)c(F)c3c(C)c2c1 ; Cc1nc2c(C)ccc(C)c2c(O)c1CN1CCN(c2ccccc2)CC1 ; Cc1nc2c(C)cccc2c(O)c1CN1CCN(c2ccccc2)CC1]

comestibly or biologically acceptable derivatives thereof.

30. A composition comprising a compound according to Formula (XIV):

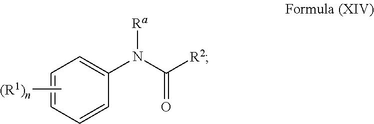

or a comestibly or biologically acceptable salt or derivative thereof, or an enantiomer or diastereomer thereof,
wherein, as valence and stability permit:
R¹, independently for each occurrence, is selected from the group consisting of C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phenyl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
R² is selected from the group consisting of C₁₋₁₀alkyl, C₁₋₁₀haloalkyl, C₂₋₁₀alkenyl, C₂₋₁₀alkynyl, hydroxyl, C₁₋₁₀alkoxy, C₆₋₁₀aryloxy, C₆₋₁₀aryloxy-C₁₋₁₀alkyl, C₆₋₁₀arylamino-C₁₋₁₀alkyl, C₆₋₁₀aryl-C₁₋₆alkyloxy, C₁₋₉heteroaryloxy, C₁₋₉heteroaryloxy-C₁₋₆alkyl, C₁₋₉heteroarylamino-C₁₋₆alkyl, C₁₋₉heteroaryl-C₁₋₆alkyloxy, C₃₋₁₀alkenyloxy, C₃₋₁₀alkynyloxy, amino, diC₁₋₁₀alkylamino, monoC₁₋₁₀alkylamino, sulfhydryl, C₁₋₁₀alkylthio, C₃₋₁₀carbocyclyl, C₃₋₁₀carbocyclyloxy, C₃₋₁₀carbocyclyl-C₁₋₆alkyl, C₃₋₁₀carbocyclyloxy-C₁₋₆alkyl, C₃₋₁₀carbocyclylamino-C₁₋₆alkyl, C₁₋₉heterocyclyl, C₁₋₉heterocyclyl-C₁₋₆alkyl, C₁₋₉heterocyclyloxy-C₁₋₆alkyl, C₁₋₉heterocyclylamino-C₁₋₆alkyl, C₆₋₁₀aryl, C₆₋₁₀aryl-C₁₋₆alkyl, C₁₋₉heteroaryl, and C₁₋₉heteroaryl-C₁₋₆alkyl, wherein heterocyclic or heteroaromatic rings, independently for each occurrence, comprise 1-4 heteroatoms selected from N, O, and S;
R^{a} is selected from the group consisting of hydrogen, C₁₋₁₀alkyl, C₁₋₁₀haloalkyl, C₂₋₁₀alkenyl, C₂₋₁₀alkynyl, carboxyl, C₁₋₁₀alkoxycarbonyl, C₂₋₁₀alkenyloxycarbonyl, C₂₋₁₀alkynyloxycarbonyl, C₁₋₁₀acyl, phosphoryl, phosphonate, phosphinate, cyano, sulfonate, sulfamoyl, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
wherein any of R¹, R², and R^{a}, independently and independently for each occurrence, is optionally substituted with 1-3 substituents selected from the group consisting of C₁₋₁₀alkyl, C₁₋₁₀haloalkyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, C₆₋₁₀aryloxy, C₆₋₁₀arylamino, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl; and wherein heterocyclic or heteroaromatic rings, independently for each occurrence, comprise 1-4 heteroatoms selected from N, O, and S; and
n is 0-3;
wherein the composition is edible and capable of reducing bitter taste of a bitter tastant.

31. The composition according to paragraph 30, wherein as valence and stability permit:
R¹, independently for each occurrence, is selected from the group consisting of halo, hydroxyl, C₁₋₆alkyl, C₁₋₆haloalkyl, C₂₋₆alkenyl, C₂₋₆alkynyl, C₁₋₆alkoxy, and C₁₋₆acyloxy;
R² is selected from the group consisting of C₁₋₆alkyl, C₁₋₆alkoxy-substituted C₁₋₆alkyl, C₆₋₁₀aryloxy-substituted C₁₋₆alkyl, C₂₋₆alkenyl, C₂₋₆alkynyl, C₆₋₁₀aryl-C₁₋₆alkyl, and —((CH₂)ₘX)ₚ—Ar, wherein aryl groups of R² are optionally substituted by one or more halo, hydroxyl, C₁₋₆alkyl, C₁₋₆haloalkyl, C₁₋₆alkoxy, or C₁₋₆acyloxy;
R^{a} is selected from the group consisting of hydrogen, C₁₋₆alkyl, C₂₋₆alkenyl, and C₂₋₆alkynyl;
X is selected from the group consisting of O, NH, and CH₂;
Ar is selected from the group consisting of C₆₋₁₀aryl, C₄₋₉heteroaryl, C5-10carbocyclyl, and C₄₋₉heterocyclyl, including fused bicyclic groups, wherein Ar is optionally substituted by one or more halo, hydroxyl, C₁₋₆alkyl, C₁₋₆haloalkyl, C₁₋₆alkoxy, or C₁₋₆acyloxy;
wherein any of R¹, R², and R^{a}, independently and independently for each occurrence, is optionally further substituted as in paragraph 30;
m is 1-3;
n is 0-3; and
p is 0 or 1.

32, The composition according to paragraph 30, wherein said compound according to Formula (XIV) is a compound according to Formula (XVa):

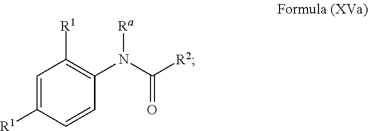

or a comestibly or biologically acceptable salt or derivative thereof, or an enantiomer or diastereomer thereof,
wherein, as valence and stability permit:
R¹ and R^{a} are as defined in paragraph 30; and
R² is C₁₋₆alkyl, such as methyl or ethyl.

33. The composition according to paragraph 30, wherein said compound according to Formula (XIV) is a compound according to Formula (XVb):

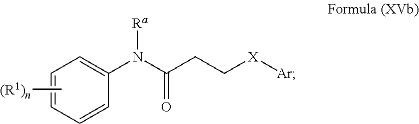

a comestibly or biologically acceptable salt or derivative thereof, or an enantiomer or diastereomer thereof, wherein, as valence and stability permit, R¹, R^{a}, X, Ar, and n are as defined in paragraph 30.

34. The composition according to paragraph 30, wherein said compound according to Formula (XIV) is a compound according to Formula (XVc):

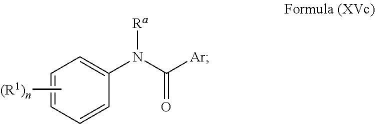

or a comestibly or biologically acceptable salt or derivative thereof, or an enantiomer or diastereomer thereof, wherein, as valence and stability permit, R¹, R², Ar, and n are as defined in paragraph 30.

35. The composition according to paragraph 30, wherein said compound according to Formula (XIV) is selected from the group consisting of:

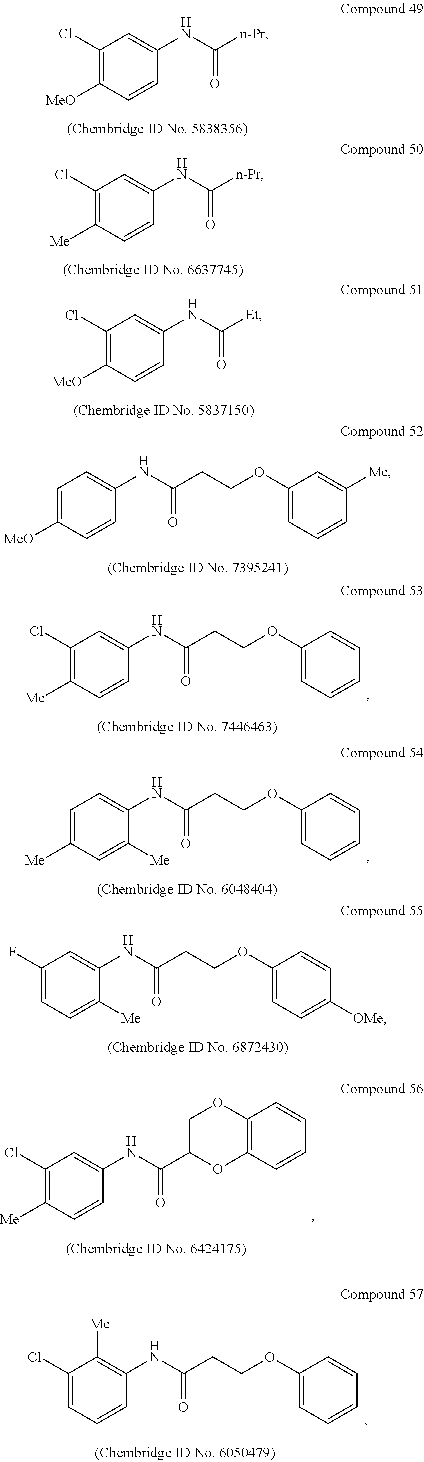

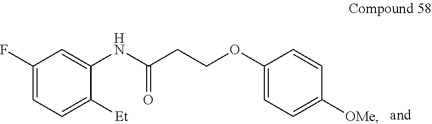 [SMILES: CCc1ccc(F)cc1NC(=O)CCOc1ccc(OC)cc1]

comestibly or biologically acceptable derivatives thereof.

36. A composition comprising:
a) a compound according to Formula (I), formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described herein, or combinations thereof; and
(b) a bitter tastant,
wherein the composition is edible.

37. The composition according to paragraph 36, wherein the bitter tastant is a foodstuff.

38. The composition according to paragraph 36, wherein the bitter tastant is a bitter tasting salt.

39. The composition according to paragraph 38, wherein the bitter tasting salt is a magnesium salt, a calcium salt, or a potassium salt.

40, The composition according to paragraph 40, wherein the potassium containing salt is KCl or potassium lactate.

41. The composition of any one of paragraphs 1-40, wherein the edible composition further comprises one or more components selected from the group consisting of: NaCl, sodium lactate, and sugar.

42. A food product comprising the compositions of any one of paragraphs 1-41.

43. A method of preparing an edible composition comprising:
(a) providing comestibly acceptable carrier; and
(b) adding to the comestibly acceptable carrier of (a) a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described herein, or combinations thereof.

44. The method according to paragraph 43, wherein said comestibly acceptable carrier is inherently bitter.

45. The method according to paragraph 44, wherein the comestibly acceptable carrier comprises a bitter tasting salt.

46. The method according to paragraph 45, wherein the bitter tasting salt is a magnesium salt, a calcium salt, or a potassium salt.

47. The method according to paragraph 46, wherein the potassium salt is KCl or potassium lactate.

48. The method according to any one of paragraphs 43-47, wherein the edible composition further comprises one or more components selected from the group consisting of: NaCl, sodium lactate, and sugar.

49. The method according to paragraph 43, wherein the method further comprises:
(c) adding a bitter tastant.

50. The method according to paragraph 49, wherein the bitter tastant is a bitter lasting salt.

51. The method according to paragraph 50, wherein the bitter tasting salt is a magnesium salt, a calcium salt, or a potassium salt.

52, The method according to paragraph 51, wherein the potassium salt is KCl or potassium lactate.

53. The method according to any one of paragraphs 49-52, wherein the edible composition further comprises one or more components selected from the group consisting of: NaCl, sodium lactate, and sugar.

54. A method of reducing the amount of NaCl in an edible composition comprising:
(a) replacing an amount of NaCl present in an edible composition with an amount of KCl; and
(b) adding to the edible composition generated in (a) an effective amount of a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described herein, or combinations thereof.

55. The method according to paragraph 54, wherein the amount of compound added in (b) is sufficient to permit replacement of the amount of NaCl typically present in the edible composition by up to 25%.

56. The method according to paragraph 54, wherein the amount of compound added in (b) is sufficient to permit replacement of the amount of NaCl typically present in the edible composition by up to 50%.

57. The method according to paragraph 54, wherein the amount of compound added in (b) is sufficient to permit replacement of the amount of NaCl typically present in the edible composition by up to 75%.

58. The method according to paragraph 54, wherein the amount of compound added in (b) is sufficient to permit replacement of the amount of NaCl typically present in the edible composition by up to 100%.

59. The method according to any one of paragraphs 54-58, wherein the edible composition maintains a sally flavor.

60. A method of reducing the amount of sodium lactate in an edible composition comprising:
(a) replacing an amount of sodium lactate present in an edible composition with an amount of potassium lactate; and
(b) adding to the edible composition generated in (a) an effective amount of a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58 as described herein, or combinations thereof.

61. The method according to paragraph 60, wherein the amount of compound added in (b) is sufficient to permit replacement of the amount of sodium lactate typically present in the edible composition by up to 25%.

62. The method according to paragraph 60, wherein the amount of compound added in (b) is sufficient to permit replacement of the amount of sodium lactate typically present in the edible composition by up to 50%.

63. The method according to paragraph 60, wherein the amount of compound added in (b) is sufficient to permit replacement of the amount of sodium lactate typically present in the edible composition by up to 75%.

64. The method according to paragraph 60, wherein the amount of compound added in (b) is sufficient to permit replacement of the amount of sodium lactate typically present in the edible composition by up to 100,%.

65. The method according to any one of paragraphs 60-64, wherein the edible composition has the same shelf life as an edible composition comprising sodium lactate.

66. A method of reducing the amount of sugar in an edible composition comprising:
(a) replacing an amount of sugar present an edible composition with an amount of Acesulfame K; and
(b) adding to the edible composition generated in (a) an effective amount of a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described herein, or combinations thereof.

67. The method according to paragraph 66, wherein the amount of compound added in (b) is sufficient to permit replacement of the amount of sugar typically present in the edible composition by up to 25%.

68. The method according to paragraph 66, wherein the amount of compound added in (b) is sufficient to permit replacement of the amount of sugar typically present in the edible composition by up to 50%.

69. The method according to paragraph 66, wherein the amount of compound added in (b) is sufficient to permit replacement of the amount of sugar typically present in the edible composition by up to 75%.

70. The method according to paragraph 66, wherein the amount of compound added in (b) is sufficient to permit replacement of the amount of sugar typically present in the edible composition by up to 100%.

71. The method according to any one of paragraphs 66-70, wherein the edible composition maintains a sweet flavor.

72, A method of reducing the sodium intake of a subject, the method comprising:
(a) replacing an amount of a sodium salt present in an edible composition with an amount of a potassium salt; and
(b) adding to the edible composition generated in (a) an effective amount of a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described herein, or combinations thereof.

73. The method according to paragraph 72, wherein the sodium salt is NaCl and the potassium salt is KCl.

74. The method according to paragraph 72, wherein the sodium salt is sodium lactate, and the potassium salt is potassium lactate.

75. The method according to any one of paragraphs 72-74, wherein the method further comprises (c) identifying a subject in need thereof.

76. The method according to any one of paragraphs 72-75, wherein the amount of compound added in (b) is sufficient to reduce sodium intake by up to 25% by replacement with potassium.

77, The method according to any one of paragraphs 72-75, wherein the amount of compound added in (b) is sufficient to reduce sodium intake by up to 50% by replacement with potassium.

78. The method according to any one of paragraphs 72-75, wherein the amount of compound added in (b) is sufficient to reduce sodium intake by up to 75% by replacement with potassium.

79. The method according to any one of paragraphs 72-75, wherein the amount of compound added in (b) is sufficient to reduce sodium intake by up to 100% by replacement with potassium.

80. A method of reducing the sugar intake of a subject, the method comprising:
(a) replacing an amount of sugar present in an edible composition with an amount of a Acesulfame K; and
(b) adding to the edible composition generated in (a) an effective amount of a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described herein, or combinations thereof.

81. The method according to paragraph 80, wherein the method further comprises (c) identifying a subject in need thereof.

82. The method according to paragraph 80 or 81, wherein the amount of compound added in (b) is sufficient to reduce sugar intake by up to 25% by replacement with Acesulfame K.

83. The method according to paragraph 80 or 81, wherein the amount of compound added in (b) is sufficient to reduce sugar intake by up to 50% by replacement with Acesulfame K.

84. The method according to paragraph 80 or 81, wherein the amount of compound added in (b) is sufficient to reduce sugar intake by up to 75% by replacement with Acesulfame K.

85. The method according to paragraph 80 or 81, wherein the amount of compound added in (b) is sufficient to reduce sugar intake by up to 100% by replacement with Acesulfame K.

86. A method of reducing bitter taste attributed to a bitter tastant in an edible composition comprising:
(a) adding an effective amount of a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described herein, or combinations thereof, to the edible composition such that any bitter taste induced by the bitter tastant is reduced.

87. A method of reducing bitter taste attributed to a bitter tastant in an edible composition comprising:
(a) ingesting an effective amount of a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described herein, or combinations thereof, along with the edible composition such that any bitter taste induced by the bitter tastant is reduced.

88. The method according to any one of paragraphs 43-87 or 89-98, wherein the edible composition is a food product, a consumer product, or a pharmaceutical composition.

89. The method according to any one of paragraphs 86-88, wherein the bitter taste induced by the bitter tastant is reduced by up to 25%

90. The method according to any one of paragraphs 86-88, wherein the bitter taste induced by the bitter tastant is reduced by up to 50%

91. The method according to any one of paragraphs 86-88, wherein the bitter taste induced by the bitter tastant is reduced by up to 75%

92. The method according to any one of paragraphs 86-88, wherein the bitter taste induced by the bitter tastant is reduced by up to 100%

93. The method according to any one of paragraphs 86-92, wherein the bitter tastant is a bitter tasting salt.

94, The method according to paragraph 93, wherein the bitter tasting salt is a magnesium salt, a calcium salt, or a potassium salt.

95. The method according to paragraph 94, wherein the potassium salt is KCl or potassium lactate.

96. The method according to any one of paragraphs 86-95, wherein the edible composition further comprises NaCl, sodium lactate, or sugar.

97. A method of preserving an edible composition comprising:
(a) providing an edible composition; and
(b) combining with the edible composition of (a) potassium lactate and a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described herein, or combinations thereof.

98. A method of reducing the amount of sodium in an edible composition while preserving the edible composition, the method comprising:
(a) replacing an amount of sodium lactate present in an edible composition with an amount of potassium lactate; and
(b) adding to the edible composition generated in (a) an effective amount of a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described herein, or combinations thereof.

99. A method of inhibiting, reducing, or eliminating a bitter taste in a subject comprising:
(a) placing a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described herein, or combinations thereof in the oral cavity of the subject.

100. The method according to paragraph 99, wherein the bitter taste is due to a bitter tasting salt.

101. The method according to paragraph 100, wherein the bitter taste is due to a magnesium salt, a calcium salt, or a potassium salt.

102. The method according to paragraph 101, wherein the bitter taste is due to KCl or potassium lactate.

103. A pharmaceutical composition comprising:
(a) a bitter tasting pharmaceutical active ingredient; and
(b) a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described herein, or combinations thereof.

104. A pharmaceutical composition comprising:
(a) a pharmaceutical active ingredient;
(b) a bitter tastant; and
(c) a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described herein, or combinations thereof.

105. A consumer product comprising:
(a) a bitter tasting ingredient; and
(b) a compound according to Formula (I), Formula (IIa), Formula (IIb), Formula (IIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described herein, or combinations thereof.

106. A consumer product for reducing bitter taste of a bitter tastant, wherein said consumer product comprises:
(a) a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described herein, or combinations thereof.

107. A method of inhibiting a bitter taste receptor comprising:
(a) contacting the bitter taste receptor with a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof or any one of Compounds 1-58, as described herein, or combinations thereof.

108. The method according to paragraph 107, wherein the bitter taste receptor is in the oral cavity of a subject.

109. The method according to paragraph 107, wherein the bitter taste receptor is in the gastrointestinal tract of a subject.

110. The method according to paragraph 107, wherein the bitter taste receptor is present in an in vitro assay.

### BRIEF DESCRIPTION OF THE DRAWINGS

FIGS. 1A-L disclose exemplary data for solution and foodstuff taste testing of compositions comprising compounds of Formula (I) of the present invention.

FIGS. 2A-H disclose exemplary data for solution and foodstuff taste testing of compositions comprising compounds of Formula (IV) of the present invention.

FIGS. 3A-D disclose exemplary data for solution and foodstuff taste testing of compositions comprising compounds of Formula (VIII) of the present invention.

FIGS. 4A-C disclose exemplary data for solution and foodstuff taste testing of compositions comprising compounds of Formula (XI) of the present invention.

FIG. 5A-E disclose exemplary data for solution and foodstuff taste testing of compositions comprising compounds of Formula (XIV) of the present invention.

### DETAILED DESCRIPTION OF THE INVENTION

In order that the invention described herein may be fully understood, the following detailed description is set forth.

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

Throughout this specification, the word “comprise” or variations such as “comprises” or “comprising” will be understood to imply the inclusion of a stated integer or groups of integers but not the exclusion of any other integer or group of integers.

The term “acyl” refers to an alkylcarbonyl, alkenylcarbonyl, alkynylcarbonyl or arylcarbonyl substituent, wherein the alkyl, alkenyl, alkynyl or aryl portion may be optionally substituted. Examples of acyl substituents include, but are not limited to, acetyl, propionyl, butyryl and benzoyl.

The term “acyloxy” refers to an —O—C(O)R substituent, wherein R is alkyl, alkenyl, alkynyl or aryl, and wherein the alkyl, alkenyl, alkynyl or aryl portion may be optionally substituted. Examples of acyloxy groups include, but are not limited to, acetoxy, propanoyloxy, butanoyloxy, pentanoyloxy and benzoyloxy.

The term “aliphatic” refers to straight chain or branched hydrocarbons that are completely saturated or that contain one or more units of unsaturation. For example, aliphatic groups include substituted or unsubstituted linear or branched alkyl, alkenyl and alkynyl groups. Unless indicated otherwise, the term “aliphatic” encompasses both substituted and unsubstituted hydrocarbons.

The terms “alkylamide,” “alkenylamide and “alkynylamide” refer to amides of the structures alky-NR—C(═O)—, alkenyl-NR—C(O)—, and alkynyl-NR—C(O)—, wherein R may be separately defined, or R is also alkyl, alkenyl or alkynyl.

The term “alkoxy” refers to O-alkyl substituent, wherein the alkyl portion may be optionally substituted. Examples of alkoxy substituents include, but are not limited to, methoxy, ethoxy, n-propoxy, isopropoxy and n-butoxy. Also explicitly included within the scope of the term “alkoxy” are O-alkenyl or O-alkynyl groups. In all cases, the alkyl, alkene and alkyne portions may be optionally substituted.

The term “alkyl” refers to both straight and branched saturated chains containing, for example, 1-3, 1-6, 1-9, or 1-12 carbon atoms. An alkyl group may be optionally substituted.

The term “alkylthio” refers to an S-alkyl substituent, wherein the alkyl portion may be optionally substituted. Examples of alkylthio substituents include, but are not limited to, methylthio, ethylthio and isopropylthio. Also explicitly included within the scope of the term “alkylthio” are S-alkenyl or S-alkynyl groups. In all cases, the alkyl, alkene and alkyne portions may be optionally substituted.

The term “alkenyl” refers to both straight and branched saturated chains containing, for example, 2-3, 2-6, 2-9, or 2-12 carbon atoms, and at least one carbon-carbon double bond. An alkenyl group may be optionally substituted.

The term “alkynyl” refers to both straight and branched saturated chains containing, for example, 2-3, 2-6, 2-9, or 2-12 carbon atoms, and at least one carbon-carbon triple bond. An alkynyl group may be optionally substituted.

The term “aralkyl” refers to an alkyl group substituted by an aryl. Also explicitly included within the scope of the term “aralkyl” are alkenyl or alkynyl groups substituted by an aryl. Examples of aralkyl groups include benzyl and phenethyl. An aralkyl group may be optionally substituted.

The terms “artificial sweetener” and “sugar substitute” refer to a food additive that confers a sweet taste but has less caloric energy than sugar. In some instances, the caloric energy of the “artificial sweetener” or “sugar substitute” is negligible.

The term “aryl” refers to monocyclic or polycyclic aromatic carbon ring systems having five to fourteen members. Examples of aryl groups include, but are not limited to, phenyl (Ph), 1-naphthyl, 2-naphthyl, 1-anthracyl and 2-anthracyl. An aryl group may be optionally substituted.

The term “arylalkoxy” refers to a group having the structure —-O—R—Ar, where R is alkyl and Ar is an aromatic substituent. Also explicitly included within the scope of the term “arylalkoxy” are —O—R—Ar groups, wherein R is alkenyl or alkynyl. In all cases, the alkyl, alkene, alkyne and aryl portions may be optionally substituted.

The term “bitter” or “bitter taste” as used herein refers to the perception or gustatory sensation resulting following the detection of a bitter tastant. The following attributes may contribute to bitter taste: astringent, bitter-astringent, metallic, bitter-metallic, as well as off-tastes, aftertastes and undesirable tastes including but not limited to freezer-burn and card-board taste, and/or any combinations of these. It is noted that, in the art, the term “off-taste” is often synonymous with “bitter taste.” Without being limited by theory, the diversity of bitter tastes may reflect the large number of bitter receptors and the differential detection of bitter tastants by these receptors. Bitter taste as used herein includes activation of a bitter taste receptor by a bitter tastant. Bitter taste as used herein also includes activation of a bitter taste receptor by a bitter tastant followed by downstream signaling. Bitter taste as used herein also includes activation of a signaling pathway after stimulation by a bitter tastant. Bitter taste as used herein further includes perception resulting from signaling following the detection of a bitter tastant by a bitter taste receptor. Bitter taste as used herein further includes perception resulting from signaling following contacting a bitter taste receptor with a bitter tastant. Bitter taste can be perceived in the brain.

The term “bitter taste receptor” refers to a receptor, typically a cell surface receptor, to which a bitter tastant can bind. Bitter taste receptors may be present in the oral cavity, and/or throughout the gastrointestinal tract, including the stomach, intestines, and colon. Bitter receptors can also be present in vitro, such as in an assay, including but not limited to a cell based assay or a binding assay.

The term “bitter tastant,” “bitter ligand,” or “bitter compound” refers to a compound that activates or that can be detected by a bitter taste receptor and/or confers the perception of a bitter taste in a subject. A “bitter tastant” also refers to a multiplicity of compounds that combine to activate or be detected by a bitter taste receptor and/or confer the perception of a bitter taste in a subject. A “bitter tastant” further refers to a compound that is enzymatically modified upon ingestion by a subject to activate or be detected by a bitter taste receptor and/or confer the perception of a bitter taste in a subject. Because the perception of bitter taste may vary from individual to individual, some individuals may describe a “bitter tastant” as a compound which confers a different kind of bitter taste compared to the kind of bitter taste perceived for the same compound by other individuals. The term bitter tastant also refers to a compound which confers a bitter taste. Those of skill in the art can readily identify and understand what is meant by a bitter tastant. Non-limiting examples of bitter tastants or substances including foods that comprise a bitter tastant and taste bitter include coffee, unsweetened cocoa, marmalade, bitter melon, beer, bitters, citrus peel, dandelion greens, escarole, quinine, magnesium salts, calcium salts, potassium salts, KCl, potassium lactate, Acesulfame K, Brussels sprouts, asparagus, bitter gourd, wild cucumber, celery, hops, kohlrabi, radish leaf, ginseng, pumpkin, collard greens, kale, sparteine, caffeine, atropine, nicotine, urea and strychnine.

Further examples of bitter tastants include pharmaceuticals. Non-limiting examples of pharmaceuticals as bitter tastants include acetaminophen, ampicillin, azithromycin, chlorpheniramine, cimetidine, dextromethorphan, diphenhydramine, erythromycin, ibuprofen, penicillin, phenylbutazone, pseudoephedrine, ranitidine, spironolactone and theophyllineall of which have been associated with bitter taste.

The term “carbocyclyl” or “carbocyclic,” refers to monocyclic or polycyclic non-aromatic carbon ring systems, which may contain a specified number of carbon atoms, preferably from 3 to 12 carbon atoms, which are completely saturated or which contain one or more units of unsaturation. A carbocyclic ring system may be monocyclic, bicyclic or tricyclic. A carbocyclyl ring may be fused to another ring, such as an aryl ring or another carbocyclic ring. Examples of carbocyclic rings could include cyclohexyl, cyclopentyl, cyclobutyl, cyclopropyl, cyclohexenyl, cyclopentenyl, indanyl, tetrahydronaphthyl and the like. The term “carbocyclic” or “carbocyclyl,” whether saturated or unsaturated, also refers to rings that are optionally substituted unless indicated. The term “carbocyclic” or “carbocyclyl” also encompasses hybrids of aliphatic and carbocyclic groups, such as (cycloalkyl)alkyl, (cycloalkenyl)alkyl and (cycloalkyl)alkenyl.

The term “comestibly or biologically acceptable salt” refers to any comestibly or biologically acceptable salt, ester, or salt of such ester, of a compound of the present invention, which, upon ingestion, is capable of providing (directly or indirectly) a compound of the present invention, or a metabolite, residue or portion thereof, characterized by the ability to reduce the perception of a bitter taste attributed to a bitter tastant. Similarly, the term “comestibly or biologically acceptable derivative” refers to any comestibly or biologically acceptable derivative of a compound of the present invention, which, upon ingestion, is capable of providing (directly or indirectly) a compound of the present invention, or a metabolite, residue or portion thereof, characterized by the ability to reduce the perception of a bitter taste attributed to a bitter tastant. A “comestible product” is a product suitable for oral use, such as eating or drinking. Therefore, a comestibly acceptable compound is an edible compound.

The term “consumer product” refers to health and beauty products for the personal use and/or consumption by a subject. Consumer products may be present in any form including, but not limited to, liquids, solids, semi-solids, tablets, capsules, lozenges, strips, powders, gels, gums, pastes, slurries, syrups, aerosols and sprays. Non-limiting examples of consumer products include nutraceuticals, nutritional supplements, lipsticks, lip balms, soaps, shampoos, gums, adhesives (e.g., dental adhesives), toothpastes, oral analgesics, breath fresheners, mouthwashes, tooth whiteners, and other dentifrices.

The term “diet” collectively refers to the food products and/or beverages consumed by a subject. A subject's “diet” also includes any consumer products or pharmaceutical compositions the subject ingests.

The term “edible composition” refers to a composition suitable for consumption, typically via the oral cavity (although consumption may occur via non-oral means such as inhalation). Edible compositions may be present in any form including, but not limited to, liquids, solids, semi-solids, tablets, lozenges, powders, gels, gums, pastes, slurries, syrups, aerosols and sprays. As used herein, edible compositions include food products, pharmaceutical compositions, and consumer products. The term edible compositions also refers to, for example, dietary and nutritional supplements. As used herein, edible compositions also include compositions that are placed within the oral cavity but not swallowed, including professional dental products, such as dental treatments, fillings, packing materials, molds and polishes. The term “comestible” refers to similar compositions and is generally used as a synonym to the term “edible.”

The term “effective amount” refers to an amount sufficient to produce a desired property or result. For example, an effective amount of a compound of the present invention is an amount capable of reducing the perception of bitter taste associated with a bitter tastant. The term “effective amount” of a compound of the invention also refers to an amount which, when added to an edible composition, reduces the bitter taste of, e.g., a NaCl substitute, thereby allowing for the maintenance of the perception of a desired salty flavor of a said edible composition. The term “effective amount of a compound” also refers to an amount which, when added to an edible composition, allows for the preservation of a food product, while reducing or eliminating bitter taste associated with a bitter tastant in the preservative. The term “effective amount” also refers to the amount of a compound of the present invention capable or reducing or eliminating the perception of a bitter taste or aftertaste associated with either a bitter tastant in a food product or an inherently bitter food product.

The term “flavor modifier” refers to a compound or a mixture of compounds that, when added to an edible composition, such as a food product, modifies (e.g., masks, eliminates, decreases, reduces or enhances the perception of) a flavor (e.g., sweet, salty, umami, sour, or bitter taste) present in the edible composition.

The term “food product” refers to any compositions comprising one or more processed foodstuff. Food products include, but are not limited to, confectionaries, bakery products (including, but not limited to, doughs, breads, cakes, biscuits, crackers, pastries, pies, tarts, quiches, and cookies), ice creams (including but not limited to impulse ice cream, take-home ice cream, frozen yogurt, gelato, sorbet, sherbet and soy, oat, bean and rice-based ice cream), dairy products (including, but not limited to, drinking milk, cheese, yogurt, and sour milk drinks), cheeses (including, but not limited to, natural cheeses and processed cheeses), butter, margarine, sweet and savory snacks (including but not limited to fruit snacks, chips/crisps, tortilla/corn chips, popcorn, pretzels, chocolates, and nuts), hot and cold beverages (including, but not limited to, beverages, beverage mixes, concentrates, juices, carbonated beverages, non-carbonated beverages, alcoholic beverages, non-alcoholic beverages, soft drinks, sports drinks, isotonic drinks, coffees, teas, bottled waters, and beverages prepared from botanicals and botanical extracts (including cold beverages that are prepared with botanical or fungi extracts as ingredients, and drinks that are prepared in various ways, such as infusions, decoctions, or other means of extraction or distillation of various plant parts, including, but not limited to leaves, flowers, sterns, fruits, roots, rhizomes, stems, bark, volatile oils, or even the whole plant)), snack bars (including, but not limited to granola bars, muesli bars, protein bars, breakfast bars, energy bars, and fruit bars), meal replacement products, ready meals (including, but not limited to canned meals, preserved meals, frozen meals, dried meals, chilled meals, dinner mixes, frozen pizza, chilled pizza, and prepared salads), soups (including but not limited to broth-like soups and cream-based soups), broth, gravy, soy sauce, meats and fish (including raw, cooked, and dried meats), deli products (including but not limited to meats and cheeses suitable for slicing or pre-sliced meats and cheeses, e.g., turkey, chicken, ham, bologna, salami, bierwurst, capicola, chorizo, corned beef, dutch loaf, Serrano, prosciutto, head cheese, liverwurst, meatloaf (including olive loaf, pepper loaf, pimento loaf, and ham and cheese loaf), mortadella, pastrami, pepperoni, roast beef roast pork, saucisson, smoked meat, summer sausage, tongue, American cheese, blue cheese, cheddar cheese, Colby cheese, Colby-Jack cheese, gouda, Monterey Jack cheese, muenster cheese mozzarella, parmigiano cheese, pepper jack cheese, provolone, romano cheese, string cheese, spray cheese, and swiss cheese), vegetables (including, but not limited to, raw, pickled, cooked, and dried vegetables, such as french fries), fruits (including raw, cooked, and dried fruits), grains (including, but not limited to, dried cereals and breads), prepared foods (including, but not limited to, dried, canned, or jarred sauces and soups), snack foods, pastas (including, but not limited to, fresh pasta, chilled pasta, frozen pasta, dried pasta), noodles (including, but not limited to, egg noodles, wheat noodles, rice noodles, mung bean noodles, potato noodles, buckwheat noodles, corn noodles, cellophane noodles, chow mein, fettuccini, fusilli, gnocchi, lasagna, linguini, lo mein, macaroni, manicotti, pad that, penne, ramen, rice vermicelli, rigatoni, soba, spaghetti, spatzle, udon, and ziti), canned foods, frozen foods, dried foods, chilled foods, oils and fats, baby food, spreads, salads, cereals (including, but not limited to, hot and cold cereals), sauces (including, but not limited to, tomato pastes, tomato purees, bouillon cubes, stock cubes, table sauces, boys bases sauces, pasta sauces, cooking sauces, marinades, dry sauces, powder mixes, ketchups, mayonnaises, salad dressings, vinegrettes, mustards, and dips), jellies, jams, preserves, honey, puddings, recipe mixes, syrups, icings, fillings, infused foods, salt-preserved food, marinated foods and condiments (such as ketchup, mustard and steak sauce). In some embodiments, the food product is animal feed. For example, the food product may be a pet food product, i.e. a food product for consumption by a household pet. In other embodiments, the food product is a livestock food product, i.e. a food product for consumption by livestock.

The term “foodstuff” refers to an unprocessed ingredient or a basic nutrient or flavor containing element used to prepare a food product. Non-limiting examples of foodstuffs include: fruits, vegetables, meats, fishes, grains, milks, eggs, tubers, sugars, sweeteners, oils, herbs, snacks, sauces, spices and salts.

The term “halo” or “halogen” refers to a fluorine, chlorine, bromine or iodine substituent.

The term “heteroaryl” refers to monocyclic or polycyclic aromatic ring systems having five to fourteen members and one or more heteroatoms. One having ordinary skill in the art will recognize that the maximum number of heteroatoms in a stable, chemically feasible heteroaryl ring is determined by the size of the ring and valence. The term “heteroaralkyl” refers to an alkyl group substituted by a heteroaryl. Also explicitly included within the scope of the term “heteroaralkyl” are alkenyl or alkynyl groups substituted by a heteroaryl. In general, a heteroaryl ring may have one to four heteroatoms. Heteroaryl groups include, without limitation, 2-furanyl, 3-furanyl, N-imidazolyl, 2 imidazolyl, 4-imidazolyl, 5-imidazolyl, 3-isoxazolyl, 4 isoxazolyl, 5-isoxazolyl, 2-oxadiazolyl, 5-oxadiazolyl, 2-oxazolyl, 4-oxazolyl, 5-oxazolyl, 2-pyrrolyl, 3-pyrrolyl, 2-pyridyl, 3-pyridyl, 4-pyridyl, 2-pyrimidyl, 4-pyrimidyl, 5-pyrimidyl, 3-pyridazinyl, 2-thiazolyl, 4thiazolyl, 5-thiazolyl, 5-tetrazolyl, 2-triazolyl, 5-triazolyl, 2-thienyl, and 3-thienyl. The term “heteroaryl ring”, “heteroaryl group”, or “heteroaralkyl” also refers to rings that are optionally substituted. Examples of fused polycyclic heteroaryl and aryl ring systems in which a carbocyclic aromatic ring or heteroaryl ring is fused to one or more other rings include, tetrahydronaphthyl, benzimidazolyl, benzothienyl, benzofuranyl, indolyl, quinolinyl, benzotriazolyl, benzoxazolyl, benzimidazolyl, isoquinolinyl, isoindolyl, acridinyl, benzoisoxazolyl, and the like.

The term “heterocyclic” or “heterocyclyl” refers to non-aromatic saturated or unsaturated monocyclic or polycyclic ring systems containing one or more heteroatoms and with a ring size of three to fourteen. One having ordinary skill in the art will recognize that the maximum number of heteroatoms in a stable, chemically feasible heterocyclic ring is determined by the size of the ring, degree of unsaturation, and valence. In general, a heterocyclic ring may have one to four heteroatoms so long as the heterocyclic ring is chemically feasible and stable and may be fused to another ring, such as a carbocyclic, aryl or heteroaryl ring, or to another heterocyclic ring. A heterocyclic ring system may be monocyclic, bicyclic or tricyclic. Also included within the scope of within the scope of the term “heterocyclic” or “heterocyclyl”, as used herein, is a group in which one or more carbocyclic rings are fused to a heteroaryl. Examples of heterocyclic rings include, but are not limited to, 3-1H-benzimidazol-2-one, 3-1H-alkyl-benzimidazol-2-one, 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, dazolonyl, N-substituted dazolonyl, 1-phthalimidinyl, benzoxane, benzotriazol-1-yl, benzopyrrolidine, benzopiperidine, benzoxolane, benzothiolane, benzothiane, aziranyl, oxiranyl, azetidinyl, pyrrolinyl, dioxolanyl, imidazolinyl, imidazolidinyl, pyrazolinyl, pyrazolidinyl, pyranyl, dioxanyl, dithianyl, trithianyl, quinuclidinyl, oxepanyl, succinimidyl and thiepanyl.

The term “isoprene” (also referred to as “isoterpene”) refers to 2-methyl-1,3-butadiene and is represented by the formula CH₂═C(CH₃)CH═CH₂.

The terms “parts per million” and “ppm” are used in the food industry to refer to a low concentration of a solution. For example, one gram of solute in 1000 ml of solvent has a concentration of 1000 ppm and one thousandth of a gram (0.001 g) of solute in 1000 ml of solvent has a concentration of one ppm. Accordingly, a concentration of one milligram per liter (i.e. 1 mg/L) is equal to 1 ppm.

The terms “perception of a bitter taste,” “perception of saltiness,” “perception of a flavor” and similar terms, refer to the awareness of a subject of a particular taste or flavor.

The term “pharmaceutically active ingredient” refers to a compound in a pharmaceutical composition which is biologically active.

The term “potassium salt” refers to a salt wherein potassium is the cation. Potassium salts in the context of the present invention are preferably edible potassium salts including, but not limited to, Acesulfame K (Ace K), aluminum potassium sulfate, dipotassium guanylate, dipotassium inosinate, monopotassium glutamate, potassium acetate, potassium acid tartate, potassium acid tartrate, potassium adipate, potassium alginate, potassium aluminum silicate, potassium ascorbate, potassium aspartate, potassium benzoate, potassium bicarbonate, potassium bisulfate, potassium bisulfite, potassium bromate, potassium carbonate, potassium chloride, potassium citrate, potassium dihydrogen citrate, potassium dihydrogen phosphate, potassium ferrocyanide, potassium fumarate, potassium gibberellate, potassium gluconate, potassium hydroxide, potassium hydrogen sulfite, potassium iodide, potassium lactate, potassium malate, potassium metabisulfite, potassium nitrate, potassium nitrite, potassium persulfate, potassium phosphate (dibasic), potassium phosphate (monobasic), potassium phosphate (tribasic), potassium polymetaphosphate, potassium polyphosphates, potassium pyrophosphate, potassium propionate, potassium saccharin, potassium sodium tartrate (e.g., potassium sodium L(+)-tartrate), potassium sorbate, potassium sulfate, potassium sulfite, and potassium tripolyphosphate.

The term “processed foodstuff” refers to a foodstuff has been subjected to any process which alters its original state (excluding, e.g., harvesting, slaughtering, and cleaning). Examples of methods of processing foods include, but are not limited to, removal of unwanted outer layers, such as potato peeling or the skinning of peaches; choppmg or slicing; mincing or macerating; liquefaction, such as to produce fruit juice; fermentation (e.g. beer); emulsification; cooking, such as boiling, broiling, frying, heating, steaming or grilling; deep frying; baking; mixing; addition of gas such as air entrainment for bread or gasification of soft drinks; proofing; seasoning (with, e.g., herbs, spices, salts); spray drying; pasteurization; packaging (e.g., canning or boxing); extrusion; puffing; blending; and preservation (e.g., adding salt, sugar, potassium lactate or other preservatives).

The term “replace” or “replacing” refers to substituting one compound for another compound in or in the preparation of, for example, an edible composition, such as food product. It includes complete and partial replacements or substitutions.

The term “salty flavor” refers to the taste elicited by, for example, ions of alkali metals salts (e.g., Na⁺ and Cl⁻ in sodium chloride). Non-limiting examples of compositions eliciting a salty flavor include table salt (sodium chloride), sea water, sea salt and potassium chloride. The amount of salty flavor or the saltiness of a composition can be determined by, e.g., taste testing.

The term “sodium” or “sodium salt” refers to the amount of sodium (i.e., sodium salt) ingested or otherwise consumed by a subject. In general, “sodium” or a “sodium salt” refers to a salt or compound wherein sodium is the cation. Sodium salts in the context of the present invention include, but are not limited to aluminium sodium sulfate, calcium disodium EDTA, dioctyl sodium sulfosuccinate, disodium 5′-ribonucleotides, disodium ethylenediaminetetraacetate, disodium guanylate, disodium inosinate sodium acetate, monosodium glutamate (MSG), potassium sodium tartrate, sodium acid pyrophosphate, sodium adipate, sodium alginate, sodium aluminosilicate, sodium aluminum phosphate (acidic), sodium aluminum phosphate (basic), sodium ascorbate, sodium benzoate, sodium bicarbonate, sodium bisulfate, sodium bisulfite, sodium carbonate, sodium carboxymethylcellulose, sodium caseinate, sodium chloride, sodium citrate, sodium cyclamate, sodium dehydroacetate, sodium diacetate, sodium dehydroacetate, sodium dihydrogen citrate, sodium dihydrogen phosphate, sodium DL-malate, sodium erythorbate, sodium erythorbin, odium ethyl para-hydroxybenzoate, sodium ferric pyrophosphate, sodium ferrocyanide, sodium formate, sodium fumarate, sodium gluconate, sodium hydrogen carbonate, sodium hydrogen DL-malate, sodium hydrogen acetate, sodium hydrogen sulfite, sodium hydroxide, sodium hypophosphite, sodium tartrate (e.g., sodium L(+)-tartrate), sodium lactate, sodium lauryl sulfate, sodium malate, sodium metabisulfite, sodium metaphosphate, sodium methyl para-hydroxybenzoate, sodium nitrate, sodium nitrite, sodium O-phenylphenol, sodium phosphate (dibasic), sodium phosphate (monobasic), sodium phosphate (tribasic), sodium polyphosphate, sodium potassium tartrate, sodium propionate, sodium propyl para-hydroxybenzoate, sodium pyrophosphate, sodium saccharin, sodium sesquicarbonate, sodium stearoyl lactylate, sodium stearyl fumarate, sodium succinate, sodium sulfate, and starch sodium octenylsuccinate.

The term “sodium intake” refers to the amount of sodium ingested or otherwise consumed by a subject.

The term “stability” or “stable” in the context of a chemical structure refers to the chemical state when a system is in its lowest energy state, or in chemical equilibrium with its environment. Thus, a stable compound (or, e.g., a compound containing a number of atoms or substitutions that are stable) is not particularly reactive in the environment or during normal use, and retains its useful properties on the timescale of its expected usefulness.

The term “subject” refers to a mammal. In preferred embodiments, the subject is human. In some embodiments, a subject is a domestic or laboratory animal, including but not limited to, household pets, such as dogs, cats, pigs, rabbits, rats, mice, gerbils, hamsters, guinea pigs, and ferrets. In some embodiments, a subject is a livestock animal. Non-limiting examples of livestock animals include: alpaca, bison, camel, cattle, deer, pigs, horses, llamas, mules, donkeys, sheep, goats, rabbits, reindeer, and yak.

The term “sugar” refers to a simple carbohydrate, such as a monosaccharide or a disaccharide, that delivers a primary taste sensation of sweetness. Non-limiting examples of sugar include glucose, fructose, galactose, sucrose, lactose, and maltose.

The term “sweet flavor” refers to the taste elicited by, for example, sugars. Non-limiting examples of compositions eliciting a sweet flavor include glucose, sucrose, fructose, saccharin, cyclamate, aspartame, acesulfame potassium, sucralose, alitame, and neotame. The amount of sweet flavor or the sweetness of a composition can be determined by, e.g., taste testing.

The term “terpenes” refers to compounds comprising repeating units of isoprene. The basic molecular formula of a terpene is (C₅H₈)ₙ where n is the number of linked isoprene units.

The term “terpeneoids” refers to compounds comprising terpenes and derivatives thereof. Thus, in some embodiments, terpenoids have at least one C₅H₈ hydrocarbon unit with one or more points of unsaturation. In other embodiments, terpenoids comprise saturated terpene unites and derivatives thereof and have no points of unsaturation.

An aryl, aralkyl, heteroaryl, or heteroaralkyl group may contain one or more independently selected substituents. Examples of suitable substituents on the unsaturated carbon atom of an aryl or heteroaryl group include, but are not limited to, halogen, —CF₃, —R′, —OR′, —OH, —SH, —SR′, protected OH (such as acyloxy), —NO₂, —CN, —NH₂, —NHR′, —N(R′)₂, —NHCOR′, —NHCONH₂, —NHCONHR′, —NHCON(R′)₂, —NRCOR′, —NHCO₂H, —NHCO₂R′, —CO₂R′, —CO₂H, —COR′, —CONH₂, —CONHR′, —CON(R′)₂, —S(O)₂H, —S(O)₂R′, —S(O)₃H, —S(O)R′, —S(O)NH2′—S(O)H, —S(O)R′, —S(O)₂NHR′, —S(O)₂N(R′)₂, —NHS(O)₂H, or —NHS(O)₂R′, where R′ is selected from H, aliphatic, carbocyclyl, heterocyclyl, aryl, aralkyl, heteroaryl, or heteroaralkyl and each R′ is optionally substituted with one or more halogen, nitro, cyano, amino, —NH-(unsubstituted aliphatic), —N-(unsubstituted aliphatic)₂, carboxy, carbamoyl, hydroxy, —O-(unsubstituted aliphatic), —SH, —S-(unsubstituted aliphatic), CF₃, —S(O)₂NH₂′ unsubstituted aliphatic, unsubstituted carbocyclyl, unsubstituted heterocyclyl, unsubstituted aryl, unsubstituted aralkyl, unsubstituted heteroaryl, or unsubstituted heteroaralkyl.

An aliphatic group, a carbocyclic ring or a heterocyclic ring may contain one or more substituents. Examples of suitable substituents on a saturated or unsaturated carbon of an aliphatic group, a carbocyclic ring or a heterocyclic ring include, but are not limited to, those listed above for the unsaturated carbon as well as the following: ═O, ═S, ═NNHR′, ═NN(R′)₂, ═N—OR′, —NNHCOR′, ═NNHCO₂R′, —NNHSO₂R′, ═N—CN, or ═NR′, wherein R′ is as defined above. Guided by this specification, the selection of suitable substituents is within the knowledge of one skilled in the art.

As defined herein, the compounds of the invention are intended to include all stereochemical forms of the compound, including geometric isomers (i.e., E, Z) and optical isomers (i.e., R, S). Single stereochemical isomers as well as enantiomeric and diastereomeric mixtures of the present compounds are within the scope of the invention. Unless otherwise stated, formulas depicted herein are also meant to include compounds which differ only in the presence of one or more isotopically enriched atoms. For example, compounds having the present formulas except for the replacement of a hydrogen by a deuterium or tritium, or the replacement of a carbon by a ¹³C- or ¹⁴C-enriched carbon are within the scope of this invention.

The present invention provides edible compositions comprising a compound of the present invention, including food products, consumer products, and pharmaceutical compositions comprising said compounds, and methods of preparing a such compositions. The present invention also provides methods of reducing the amount of sodium (e.g., NaCl or sodium lactate) or sugar in a food product, a method of reducing the sodium or sugar intake in a diet, a method of reducing bitter taste, and a method of reducing activity of a bitter taste receptor. The present invention also includes reducing the amount of sodium in a edible composition or diet by replacing a sodium containing compound or composition with a potassium containing compound or composition. The present invention also includes reducing the amount of sugar in a edible composition or diet by replacing sugar with a potassium containing sweetener, such as Acesulfame K.

### Edible Compositions

According to one aspect, the invention provides an edible composition comprising a compound of the invention for reducing bitter taste of a bitter tastant.

### Edible Compositions Comprising Diphenyl-Containing Compounds

The substituent definitions in this section (i.e., R¹, R², R^{a}, X, m, and n) refer to compounds of Formula (I), Formula (IIa), Formula (IIb), Formula (IIIb), Formula (IIIb′) and Formula (IIIb″).

All stereochemical forms of the compounds disclosed in this and any section herein are specifically contemplated, including geometric isomers (i.e., E, Z) and optical isomers (i.e., R, S). Single stereochemical isomers as well as enantiomeric and diastereomeric mixtures of the compounds disclosed in this and any section herein are also specifically contemplated.

In some embodiments, the present invention provides an edible composition for reducing bitter taste of a bitter tastant, wherein the composition comprises a diphenyl-containing compound. The diphenyl-containing compounds of this invention are capable of reducing or eliminating bitter taste of a bitter tastant. In some embodiments, the diphenyl-containing compound has a molecular weight less than about 1000, 500, or 300 daltons. In certain embodiments, the diphenyl-containing compound is a compound of Formula (I):

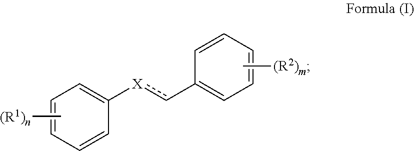

or a comestibly or biologically acceptable salt or derivative thereof, or an enantiomer or diastereomer thereof,
wherein, as valence and stability permit:
R¹, independently for each occurrence, is selected from the group consisting of C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, C₆₋₁₀aryloxy, C₆₋₁₀aryl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;

R², independently for each occurrence, is selected from the group consisting of C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, C₆₋₁₀aryloxy, C₆₋₁₀aryl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀ccarbamate, C₁₋₁₀urea, cyano, nitro, azido, suliihydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
X is O or NR^{a}, wherein R^{a} is absent or is selected from the group consisting of hydrogen, C₁₋₁₀alkyl, C₁₋₁₀haloalkyl, C₂₋₁₀alkenyl, C₂₋₁₀alkynyl, carboxyl, C₁₋₁₀-alkoxycarbonyl, C₂₋₁₀alkenyloxycarbonyl, C₂₋₁₀alkynyloxycarbonyl, C₁₋₁₀acyl, phosphoryl, phosphonate, phosphinate, cyano, sulfonate, sulfamoyl, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
wherein any of R¹, R², and R^{a}, independently and independently for each occurrence, is optionally substituted with 1-3 substituents selected from the group consisting of C₁₋₁₀alkyl, C₁₋₁₀haloalkyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl; and wherein heterocyclic or heteroaromatic rings, independently for each occurrence, comprise 1-4 heteroatomrs selected from N, O, and S;
m is 1-3: and
n is 0-3.

According to some embodiments of compounds of Formula I,
as valence and stability permit:
R¹, independently for each occurrence, is selected from the group consisting of halo; hydroxyl; C₁₋₆alkyl; C₁₋₆haloalkyl, C₁₋₆hydroxylalkyl, or C₁₋₆acyloxy-C₁₋₅alkyl; C₂₋₆alkenyl; C₂₋₆alkynyl; C₁₋₆alkoxy; C₁₋₆alkylthio; and C₆₋₁₀aryl-C₁₋₆alkyloxy optionally substituted by halo, hydroxyl, C₁₋₆alkyl, C₁₋₆alkoxy, or C₁₋₆acyloxy;
R², independently for each occurrence, is selected from the group consisting of halo; hydroxyl; C₁₋₆alkyl; C₁₋₆haloalkyl, C₁₋₆hydroxylalkyl, or C₁₋₆acyloxy-C₁₋₆alkyl; C₂₋₆alkenyl; C₂₋₆alkynyl; C₁₋₆alkoxy; C₁₋₆alkylthio; and C₆₋₁₀aryl-C₁₋₆alkyloxy optionally substituted by halo, hydroxyl, C₁₋₆alkyl, C₁₋₆alkoxy, or C₁₋₆acyloxy;
X is O or NR^{a}, wherein R^{a} is absent or is selected from the group consisting of hydrogen and C₁₋₆alkyl;
wherein any of R¹, R², and R^{a}, independently and independently for each occurrence, is optionally further substituted as noted above;
m is 1-3; and
n is 0-3.

According to some embodiments of the compound of Formula (I), X is O. In other embodiments, X is NR^{a}, wherein R^{a} is absent. For example, in certain embodiments, the compound of Formula (I) is an imine-containing comrpound. For instance, in some embodiments, the compound of Formula (I) is a compound of Formula (IIa):

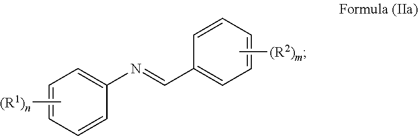

or a comestibly or biologically acceptable salt or derivative thereof, or an enantiomer or diastereomer thereof, wherein, as valence and stability permit, R¹, R², m, and n are as defined above.

In certain embodiments, one or more occurrences of R¹ is C₁₋₆alkyl, such as methyl, one or more occurrences of R is C₁₋₆hydroxylalkyl, and/or one or more occurrences of R¹ is C₁₋₆alkoxy, such as methoxy.

In some embodiments, one or more occurrences of R² is C₁₋₆alkyl, such as methyl, one or more occurrences of R² is C₁₋₆hydroxylalkyl, and/or one or more occurrences of R² is C₁₋₆alkoxy, such as methoxy.

In certain embodiments, the compound of Formula (I) or Formula (IIa) is:

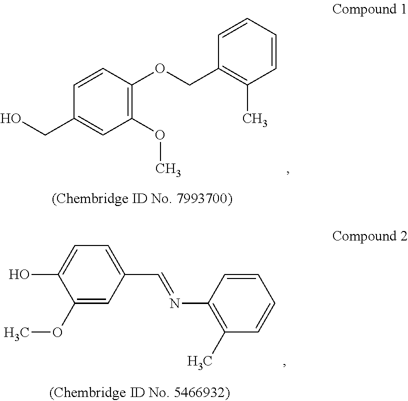 [SMILES: COc1cc(CO)ccc1OCc1ccccc1C ; COc1cc(/C=N/c2ccccc2C)ccc1O]

or a comestibly or biologically acceptable salt or derivative thereof, or an enantiomer or diastereomer thereof.

According to some embodiments of the compound of Formula (I), X is NR^{a}, wherein R^{a} is hydrogen or C₁₋₆alkyl. In particular einbodiments, R^{a} is hydrogen. For example, in certain embodiments, the compound of Formula (I) is a benzylamine compound. For instance, in some embodiments, the compound of Formula (I) is a compound of Formula (IIb):

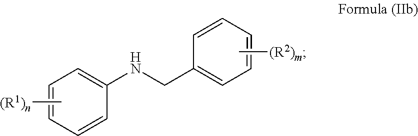

or a comestibly or biologically acceptable salt or derivative thereof, or an enantiomer or diastereomer thereof, wherein, as valence and stability permit, R¹, R², m, and n are as defined above.

In cetain embodiments, one or more occurrences of R¹ is C₁₋₆alkyl, such as methyl, one or more occurrences of R¹ is C₁₋₆alkoxy, such as methoxy or ethoxy, and/or one or more occurrences of R¹ is C₁₋₆alkylthio, such as, methylthio.

In some embodiments, one or more occurrences of R¹ is halo, such as fluoro, chloro, or bromo.

In cetain embodiments, one or more occurrences oCR¹ is hydroxyl.

In some embodiments, n is 0. In other embodiments, n is 1. For example, in some embodiments, n is 1 and R¹ is C₁₋₆alkyl, such as methyl, or R¹ is C₁₋₆alkoxy, such as methoxy. In yet other embodiments, n is 2. For example, in some embodiments, n is 2 and one or both occurrences of R¹ is C₁₋₆alkyl, such as methyl, and/or one or both occurrences of R¹ is C₁₋₆alkoxy, such as methoxy. In certain embodiments, n is 2 and one occurrence of R¹ is halo, and the other occurrence of R¹ is C₁₋₆alkyl, such as methyl.

In some embodiments, m is 1. For example, in some embodiments, m is 1 and R² is C₁₋₆alkyl, such as methyl, or R² is C₁₋₆alkoxy, such as methoxy. In yet other embodiments, m is 2. For example, in some embodiments, m is 2 and one or both occurrences of R² is C₁₋₆alkyl, such as methyl, and/or one or both occurrences of R² is C₂₋₆alkoxy, such as methoxy or ethoxy. For instance, in some embodiments, the compound of Formula (IIb) is a compound of Formula (IIIb):

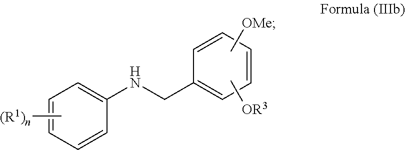

or a comestibly or biologically acceptable salt or derivative thereof, or an enantiomer or diastereomner thereof,
wherein, as valence and stability permit:
R¹ and n are as defined above; and
R³ is selected from the group consisting of methyl and ethyl.

In some embodiments of compounds of Formula (IIb), m is 2 and one or both occurrences of R² is C₁₋₆alkyl, such as methyl; one or both occurrences of R² is C₁₋₆alkoxy, such as methoxy; and/or one or both occurrences of R² is C₆₋₁₀aryl-C₁₋₆alkyloxy, such as phenyl-C₆₋₁₀alkyloxy, optionally substituted by halo, hydroxyl, C₁₋₆alkyl, C₁₋₆alkoxy, or C₁₋₆acyloxy. For instance, in some embodiments, the compound of Formula (IIb) is a compound of Formula (IIIb′):

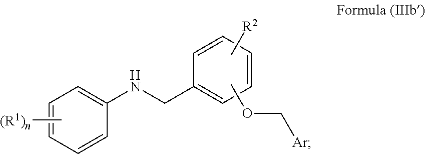

or a comestibly or biologically acceptable salt or derivative thereof, or an enantiomer or diastereomer thereof,
wherein, as valence and stability permit:
R¹, R², and n are as defined above; and
Ar is C₆₋₁₀aryl optionally substituted by halo, hydroxyl, C₁₋₆alkyl, C₁₋₆alkoxy, or C₁₋₆acyiloxy.

In some embodiments, Ar is phenyl, optionally substituted by halo, hydroxyl, C₁₋₆alkyl, C₁₋₆alkoxy, or C₁₋₆acyloxy. In certain embodiments, Ar is substituted by C₁₋₅alkyl, such as methyl.

In some embodiments for compounds of Formula (IIb), n is 1 and R¹ is C₁₋₆alkyl, such as methyl, R¹ is C₁₋₆alkoxy, such as methoxy, or R¹ is C₁₋₆alkylthio, such as methylthio. In yet other embodiments, n is 2. For example, in some embodiments, n is 2 and one or both R¹ is halo (e.g., fluoro, chloro, or bromo), one or both R is C₁₋₆alkyl, such as methyl, and/or one or both R¹ is C₁₋₆alkoxy, such as methoxy. For instance, in some embodiments, the compound of Formula (IIb) is a compound of Formula (IIIb″):

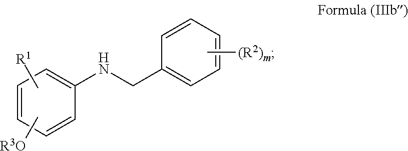

or a comestibly or biologically acceptable salt or derivative thereof, or an enantiomer or diastereomer thereof,
wherein, as valence and stability permit:
R¹, R², and m are as defined above; and
R³ is C₁₋₁₀alkyl, such as methyl.

In certain embodiments, the compound of Formula (I) is:

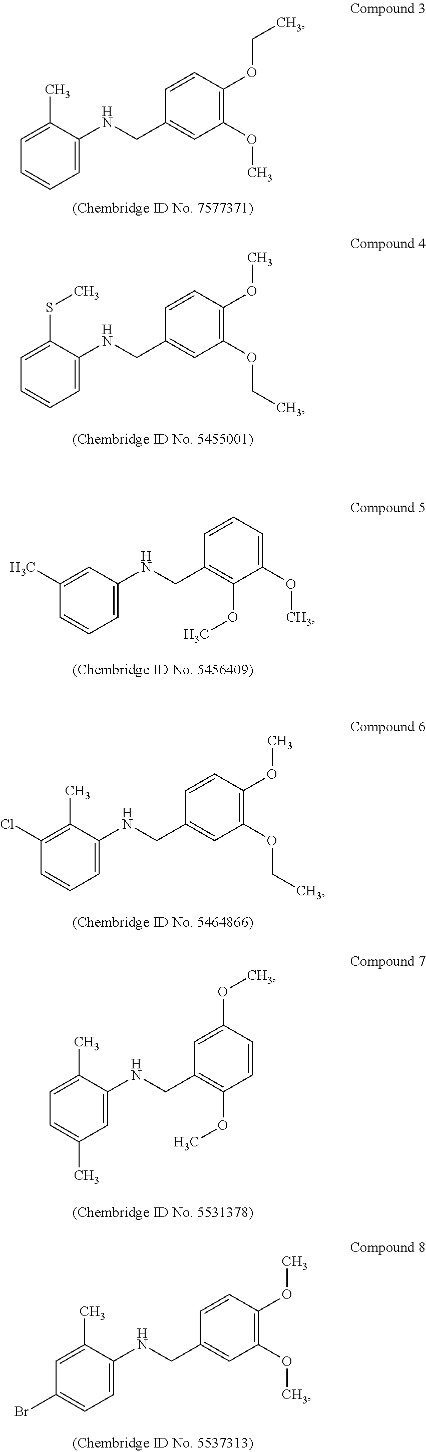 [SMILES: CCOc1ccc(CNc2ccccc2C)cc1OC ; CCOc1cc(CNc2ccccc2SC)ccc1OC ; COc1cccc(CNc2cccc(C)c2)c1OC ; CCOc1cc(CNc2cccc(Cl)c2C)ccc1OC ; COc1ccc(OC)c(CNc2cc(C)ccc2C)c1 ; COc1ccc(CNc2ccc(Br)cc2C)cc1OC]

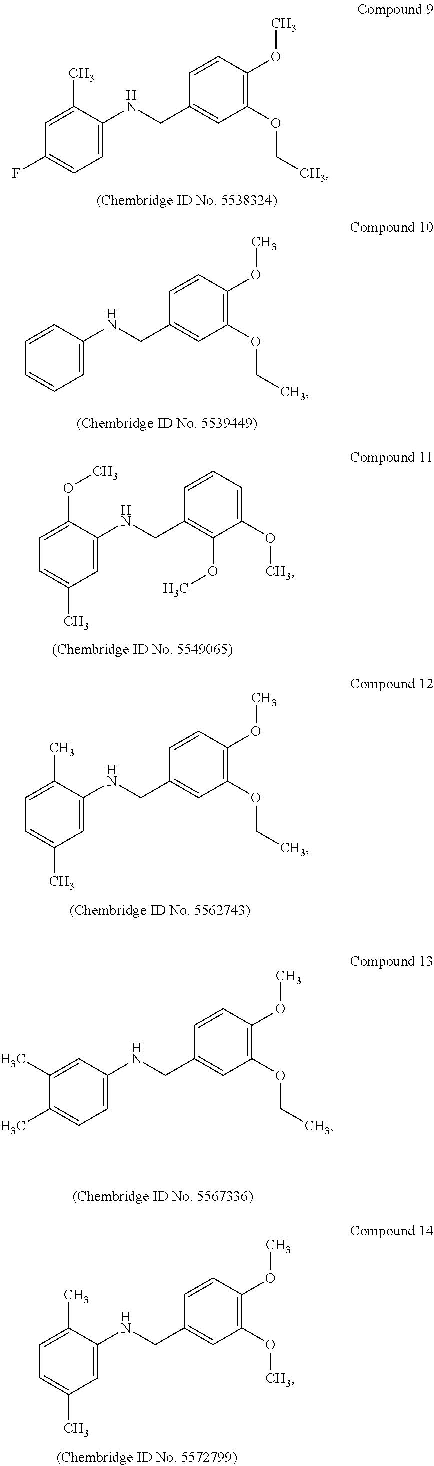 [SMILES: CCOc1cc(CNc2ccc(F)cc2C)ccc1OC ; CCOc1cc(CNc2ccccc2)ccc1OC ; COc1ccc(C)cc1NCc1cccc(OC)c1OC ; CCOc1cc(CNc2cc(C)ccc2C)ccc1OC ; CCOc1cc(CNc2ccc(C)c(C)c2)ccc1OC ; COc1ccc(CNc2cc(C)ccc2C)cc1OC]

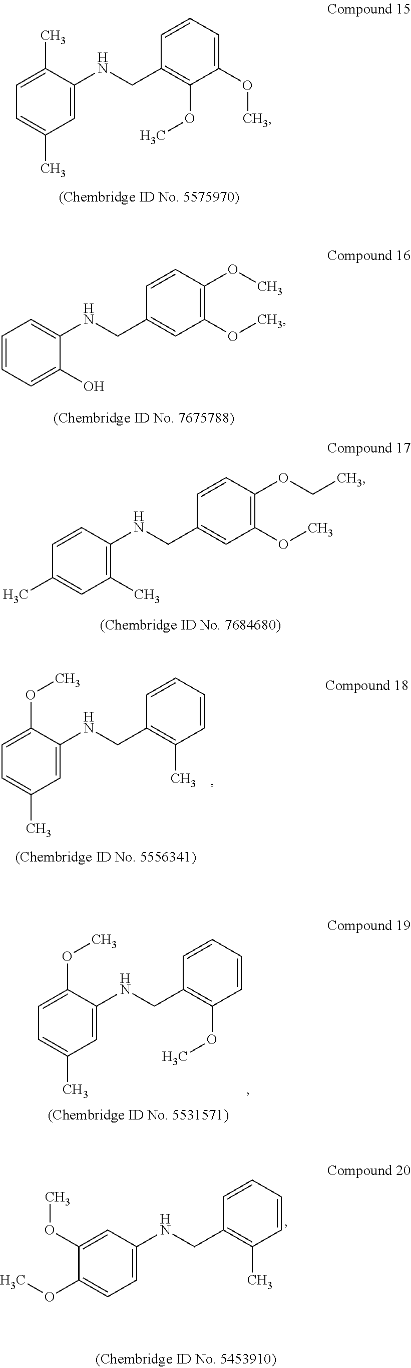 [SMILES: COc1cccc(CNc2cc(C)ccc2C)c1OC ; COc1ccc(CNc2ccccc2O)cc1OC ; CCOc1ccc(CNc2ccc(C)cc2C)cc1OC ; COc1ccc(C)cc1NCc1ccccc1C ; COc1ccccc1CNc1cc(C)ccc1OC ; COc1ccc(NCc2ccccc2C)cc1OC]

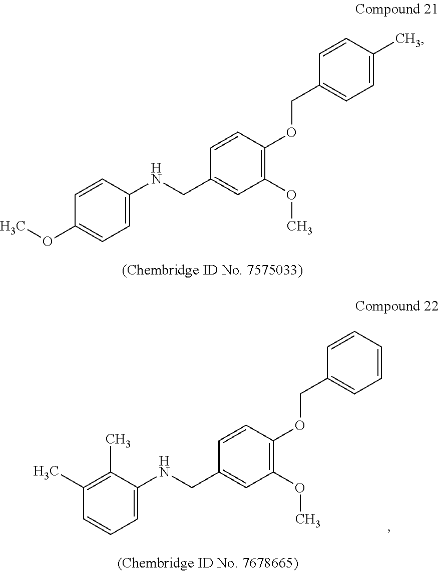 [SMILES: COc1ccc(NCc2ccc(OCc3ccc(C)cc3)c(OC)c2)cc1 ; COc1cc(CNc2cccc(C)c2C)ccc1OCc1ccccc1]

or a comestibly or biologically acceptable salt or derivative thereof, or an enantiomer or diastereomer thereof.

### Edible Compositions Comprising Pyrazole-Containing Compounds

The substituent definitions in this section (i.e. R¹, R², R³, R⁴, R⁵, m, n and o) refer to compounds of Formula (IV), Formula (Va), Formula (Vb), Formula (VIa), Formula (VIb), Formula (VIIa) and Formula (VIIb).

All stereochemical forms of the compounds disclosed in this and any section herein are specifically contemplated, including geometric isomers (i.e., E, Z) and optical isomers (i.e., R, S). Single stereochemical isomers as well as enantiomeric and diastereomeric mixtures of the compounds disclosed in this and any section herein are also specifically contemplated.

In some embodiments, the present invention provides a composition for reducing bitter taste of a bitter tastant, wherein the composition comprises a pyrazole-containing compound. The pyrazole-containing compounds of this invention are capable of reducing or eliminating bitter taste of a bitter tastant. In some embodiments, the composition is an edible composition. In some embodiments, the pyrazole-containing compound has a molecular weight less than about 1000, 500 or 300 daltons. In certain embodiments, the pyrazole-containing compound is a compound of Formula (IV):

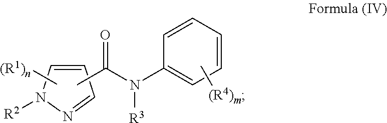

or a comestibly or biologically acceptable salt or derivative thereof, or an enantiomer or diastereomer thereof,
wherein, as valence and stability permit:
R¹, independently for each occurrence, is selected from the group consisting of C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phenyl-C₁₋₆alkyloxy, C₁₋₅heteroaryloxy, C₁₋₅heteroaryl-C₁₋₆alkyloxy, C₃₋₁₀alkenyloxy, C₁₋₅alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₅carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
R² is selected from the group consisting of hydrogen, C₁₋₁₀alkyl, C₁₋₁₀haloalkyl, C₂₋₁₀alkenyl, C₂₋₁₀alkynyl, carboxyl, C₁₋₁₀alkoxycarbonyl, C₂₋₁₀alkenyloxycarbonyl, C₂₋₁₀alkynyloxycarbonyl, C₁₋₁₀acyl, phosphoryl, phosphonate, phosphinate, cyano, sulfonate, sulfamoyl, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
R³ is selected from the group consisting of hydrogen, C₁₋₁₀alkyl, C₁₋₁₀haloalkyl, C₂₋₁₀alkenyl, C₂₋₁₀alkynyl, carboxyl, C₁₋₁₀alkoxycarbonyl, C₂₋₁₀alkenyloxycarbonyl, C₂₋₁₀alkynyloxycarbonyl, C₁₋₁₀acyl, phosphoryl, phosphonate, phosphinate, cyano, sulfonate, sulfamoyl sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₆heteroaryl, and C₁₋₅heteroaryl-C₁₋₆alkyl, wherein heterocyclic or heteroaromatic rings, independently for each occurrence, comprise 1-4 heteroatoms selected from N, O, and S;
   R⁴, independently for each occurrence, is selected from the group consisting of C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phenyl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
   wherein any of R¹, R², R³, and R⁴, independently and independently for each occurrence, is optionally substituted with 1-3 substituents selected from the group consisting of C₁₋₁₀alkyl, C₁₋₁₀haloalkyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy C₁₋₁₀carbonate, C₁₋₁₀alkoxy, phenyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₅alkyl, C₁₋₅heteroaryl, and C₁₋₅heteroaryl C₁₋₆alkyl; and wherein heterocyclic or heteroaromatic rings, independently for each occurrence, comprise 1-4 heteroatoms selected from N, O, and S;
   n is 0-2: and
   m is 0-3.

According to some embodiments of compounds of Formula IV,
as valence and stability permit:
R¹, independently for each occurrence, is selected from the group consisting of halo, C₁₋₆alkyl, C₂₋₆alkenyl, and C₂₋₆alkynyl;
R² is selected from the group consisting of hydrogen, C₁₋₆alkyl, C₂₋₆alkenyl, C₂₋₆alkynyl, and C₁₋₆acyl;
R³ is selected from the group consisting of hydrogen, C₁₋₆alkyl, C₂₋₆alkenyl, and C₂₋₆alkynyl;
R⁴, independently for each occurrence, is selected from the group consisting of halo, C₁₋₆alkyl, C₂₋₆alkenyl, C₂₋₆alkynyl, C₁₋₆alkoxy, —C(O)—O—R⁵, and —C(O)—N(R)₂;
R⁵, independently for each occurrence, is selected from the group consisting of hydrogen, C₁₋₆alkyl, C₂₋₆alkenyl, and C₂₋₆alkynyl;
wherein any of R¹, R², R³, and R⁴, independently and independently for each occurrence, is optionally substituted as noted above;
n is 0-2; and
m is 0-3.

According to some embodiments, n is 0. In other embodiments n is 1 or 2, such as 1. For example, in some embodiments, one or more occurrences of R¹ is halo, such as fluoro, chloro, bromo, or iodo. For instance, in certain embodiments, n is 1 and R¹is halo, such as fluoro, chloro, bromo, or iodo.

In certain embodiments, R² is C₁₋₆alkyl, such as methyl or ethyl.

According to certain embodiments, one or more occurrences of R⁴ is —C(O)—O—R⁵ or —C(O)—N(R⁵)₂. In some of such embodiments, R⁵is C₁₋₆alkyl, such as methyl or ethyl. For example, in some embodiments, m is 1 and R⁴ is —C(O)—O—R⁵ or —C(O)—N(R⁵)₅. In other embodiments, m is 2 and one occurrence of R⁴ is —C(O)—O—R⁵ and the other is C₁₋₆alkyl, such as methyl or ethyl, or C₁₋₆alkoxy, such as methoxy. In other embodiments, m is 3 and one occurrence of R⁴ is —C(O)—O—R⁵ and the other two occurrences are, independently, C₁₋₆alkyl, such as methyl or ethyl, C₁₋₆alkoxy, such as methoxy, or a combination thereof. In some of the above embodiments, R⁵ is hydrogen or C₁₋₆alkyl, such as methyl or ethyl.

In some embodiments, one or more occurrences of R⁴ is C₁₋₆alkyl, such as methyl or ethyl. In certain embodiments, one or more occurrences of R⁴ is C₁₋₆alkoxy, such as methoxy. In some embodiments, one or more occurrences of R⁴ is halo, such as chloro. For example, in some embodiments, m is 2 and both occurrences of R⁴ are halo, such as chloro.

In certain embodiments, the compound of Formula (IV) is a compound of Formula (Va):

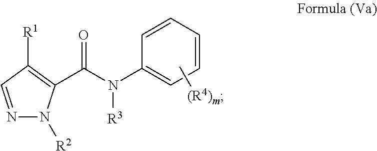

or a comestibly or biologically acceptable salt or derivative thereof, or an enantiomer or diastereomer thereof, wherein, as valence and stability permit, R¹, R², R³, R⁴, and m are as defined above.

In certain embodiments, the compound of Formula (IV) or Formula (Va) is a compound of Formula (VIa):

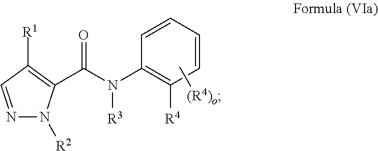

or a comestibly or biologically acceptable salt or derivative thereof, or an enantiomer or diastereomer thereof,
wherein, as valence and stability permit:
R¹, R², R³, R⁴, are as defined above; and
o is 0-2.

In certain embodiments, the compound of Formula (IV), Formula (Va), or Formula (VIa) is a compound of Formula (VIIa):

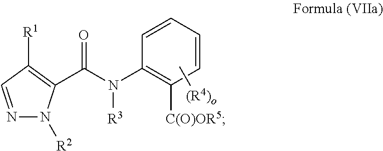

or a comestibly or biologically acceptable salt or derivative thereof, or an enantiomer or diastereomer thereof, wherein, as valence and stability permit, R¹, R², R³, R⁴, R⁵, and o are as defined above.

In certain embodiments, the compound of Formula (IV) is a compound of Formula (Vb):

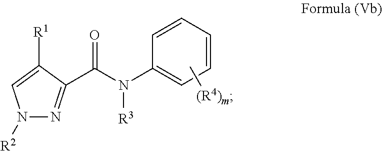

or a comestibly or biologically acceptable salt or derivative thereof or an enantiomer or diastereomer thereof, wherein, as valence and stability permit, R¹, R², R³, R⁴, and in are as defined above.

In certain embodiments, the compound of Formula (IV) or Formula (Vb) is a compound of Formula (VIb):

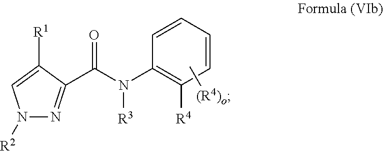

or a comestibly or biologically acceptable salt or derivative thereof, or an enantiomer or diastereomer thereof,
wherein, as valence and stability permit:
R¹, R², R³, R⁴, are as defined above; and
o is 0-2.

In certain embodiments, the compound of Formula (IV), Formula (Vb), or Formula (VIb) is a compound of Formula (VIIb):

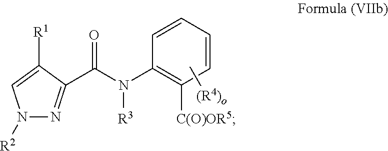

and comestibly or biologically acceptable derivatives thereof, wherein, as valence and stability permit, R¹, R², R³, R⁴, R⁵, and o are as defined above.

In certain embodiments, the compound of Formula (IV) is:

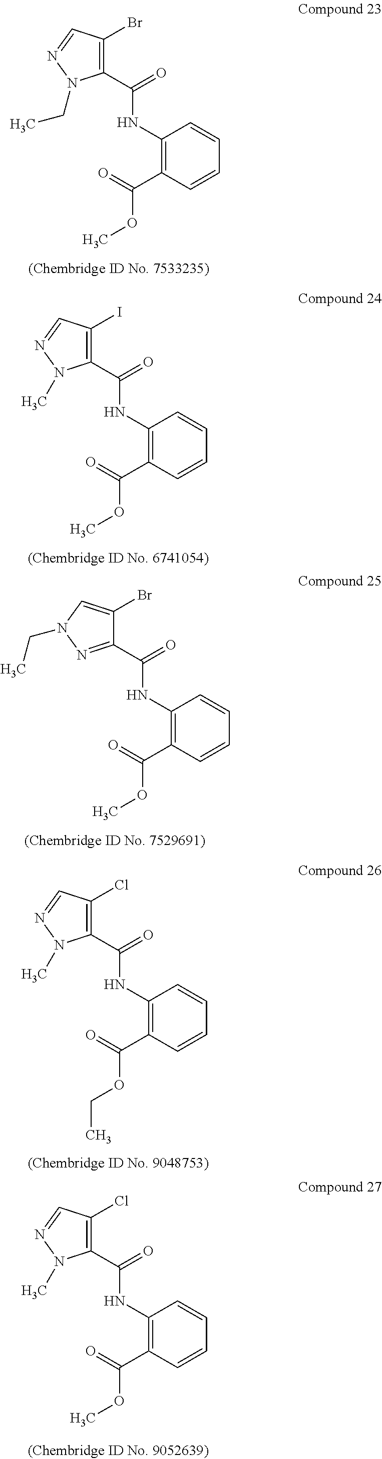 [SMILES: CCn1ncc(Br)c1C(=O)Nc1ccccc1C(=O)OC ; COC(=O)c1ccccc1NC(=O)c1c(I)cnn1C ; CCn1cc(Br)c(C(=O)Nc2ccccc2C(=O)OC)n1 ; CCOC(=O)c1ccccc1NC(=O)c1c(Cl)cnn1C ; COC(=O)c1ccccc1NC(=O)c1c(Cl)cnn1C]

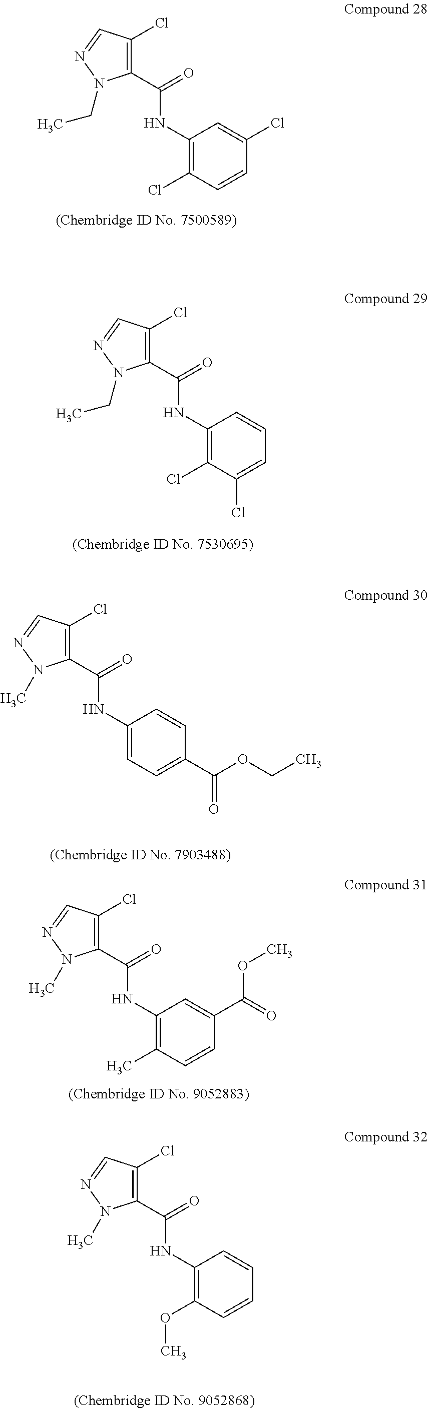 [SMILES: CCn1ncc(Cl)c1C(=O)Nc1cc(Cl)ccc1Cl ; CCn1ncc(Cl)c1C(=O)Nc1cccc(Cl)c1Cl ; CCOC(=O)c1ccc(NC(=O)c2c(Cl)cnn2C)cc1 ; COC(=O)c1ccc(C)c(NC(=O)c2c(Cl)cnn2C)c1 ; COc1ccccc1NC(=O)c1c(Cl)cnn1C]

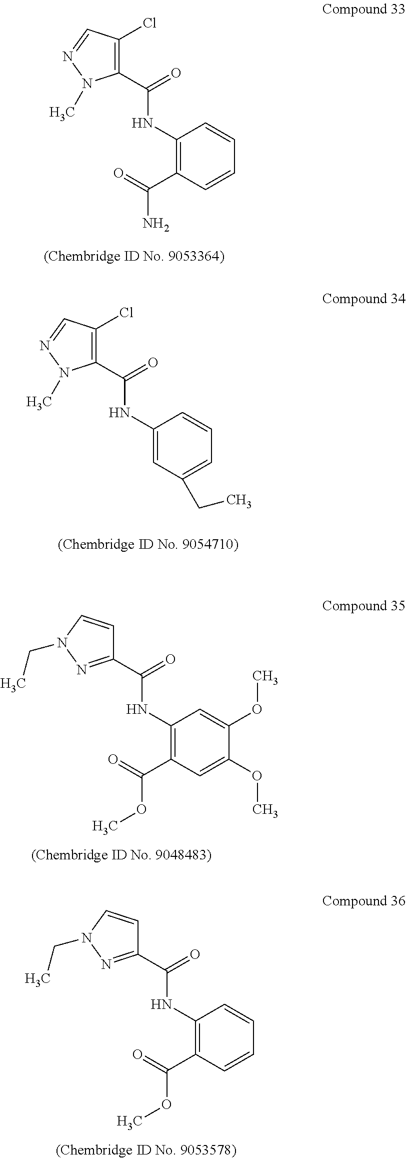

or a comestibly or biologically acceptable salt or derivative thereof, or an enantiomer or diastereomer thereof.

### Edible Compositions Comprising Hydroquinoline Compounds

The substituent definitions in this section (i.e., R¹, R², R³, R^{a}, Ar, Cy, m, n, o and p) refer to compounds of Formula (VIII), Formula (IX), and Formula (X).

All stereochemical forms of the compounds disclosed in this and any section herein are specifically contemplated, including geometric isomers (i.e., E, Z) and optical isomers (i.e., R, S). Single stereochemical isomers as well as enantiomeric and diastereomeric mixtures of the compounds disclosed in this and any section herein are also specifically contemplated.

In some embodiments, the present invention provides an edible composition for reducing bitter taste of a bitter tastant, wherein the composition comprises a hydroquinoline compound. The hydroquinoline compounds of this invention are capable of reducing or eliminating bitter taste of a bitter tastant. In some embodiments, the hydroquinoline compound has a molecular weight less than about 1000, 500, or 300 daltons. In certain embodiments, the hydroquinoline compound is a compound of Formula (VIII):

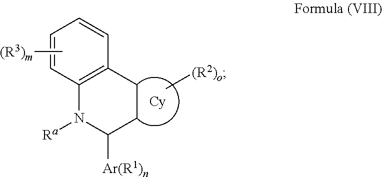

or a comestibly or biologically acceptable salt or derivative thereof, or an enantiomer or diastereomer thereof,
wherein, as valence and stability permit:
R¹, independently for each occurrence, is selected from the group consisting of C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phenyl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
R², independently for each occurrence, is selected from the group consisting of is selected from the group consisting of C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phenyl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆allyl, wherein heterocyclic or heteroaromatic rings, independently for each occurrence, comprise 1-4 heteroatoms selected from N, O, and S;
R³, independently for each occurrence, is selected from the group consisting of C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phenyl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₁₀alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
R^{a} is selected from the group consisting of hydrogen C₁₋₁₀alkyl, C₁₋₁₀haloalkyl, C₂₋₁₀alkenyl, C₂₋₁₀alkynyl, carboxyl, C₁₋₁₀alkoxycarbonyl, C₂₋₁₀alkenyloxycarbonyl, C₂₋₁₀alkynyloxycarbonyl, C₁₋₁₀acyl, phosphoryl, phosphonate, phosphinate, cyano, sulfonate, sulfamoyl, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
Ar is selected from the group consisting of C₆₋₁₀aryl and C₃₋₉heteroaryl;
Cy is a 5 to 7-membered carbocyclic or heterocyclic ring, wherein heterocyclic ring comprises 1-4 heteroatoms selected from N, O, and S;
wherein any of R¹, R², R³, and R^{a}, independently and independently for each occurrence, is optionally substituted with 1-3 substituents selected from the group consisting of C₁₋₁₀alkyl, C₁₋₁₀haloalkyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₁₀alkyl, phenyl, phenyl-C₁₋₆alkyl, C₁₋₅heteroaryl, and C₁₋₅heteroaryl-C₁₋₆alkyl; and wherein heterocyclic or heteroaromatic rings, independently for each occurrence, comprise 1-4 heteroatoms selected from N, O, and S;
m is 1-3;
n is 0-3; and
o is 0-3.

According to some embodiments of compounds of Formula VIII,
as valence and stability permit:
R¹, independently for each occurrence, is selected from the group consisting of halo, hydroxyl, C₁₋₆alkyl, C₁₋₆haloalkyl, C₂₋₆alkenyl, C₂₋₆alkynyl, C₁₋₆alkoxy, and C₁₋₆acyloxy;
R², independently for each occurrence, is C₁₋₆alkyl;
R³, independently for each occurrence, is selected from the group consisting of halo, C₁₋₆alkyl, C₂₋₆alkenyl, C₂₋₆alkynyl, C(O)—O—R⁴, and C(O)—N(R⁴)₂;
R⁴, independently for each occurrence, is selected from the group consisting of hydrogen, C₁₋alkyl, C₂₋₆alkenyl, and C₂₋₆alkynyl;
R^{a} is selected from the group consisting of hydrogen, C₁₋₆alkyl, C₂₋₆alkenyl, and C₂₋₆alkynyl;
Ar is selected from the group consisting of C₆₋₁₀aryl and C₃₋₉heteroaryl;
Cy is a 5 to 7-membered carbocyclic or heterocyclic ring, optionally including one or two carbon-carbon or carbon-nitrogen double bonds in the ring;
wherein any of R¹, R², R³, and R^{a}, independently and independently for each occurrence, is optionally substituted as noted above;
m is 1-3;
n is 0-3; and
o is 0-3.

In some embodiments, n is 0. In other embodiments, n is 1. For example, in certain embodiments, n is 1 and R¹ is halo (such as fluoro, chloro, or bromo) or C₁₋₆acyloxy (such as acetyloxy). In other embodiments, n is 2. For example, in some embodiments, n is 2 and one or both occurrences of R¹ is halo, such as chloro.

In some embodiments, m is 1. For example, in certain embodiments, m is 1 and R³ is C(O)—O—R⁴, such as C(O)—OH, C(O)—OMe, or C(O)—OEt. In other embodiments, nm is 2. For example, in some embodiments, m is 2 and one occurrence of R³ is C(O)—O—R⁴ and the other occurrence is halo, such as bromo.

In certain embodiments, o is 0. In other embodiments, o is 1-3.

In certain embodiments, R^{a} is hydrogen. In other embodiments, R^{a} is C₁₋₆alkyl, such as methyl.

In certain embodiments, Ar is C₆₋₁₀aryl, such as phenyl. In other embodiments, Ar is C₃₋₉heteroaryl.

In certain embodiments, Cy is a 5 to 7-membered carbocyclic ring, such as a 5-membered carbocyclic ring, such as a cyclopentyl or cyclopentenyl ring. In some embodiments, Cy includes one carbon-carbon double bond in the ring, such as in a cyclopentenyl ring. For instance, according to one embodiment, the compound of Formula (VIII) is a compound of Formula (IX):

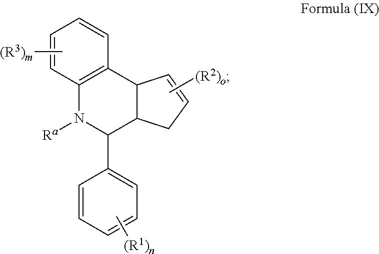

or a comestibly or biologically acceptable salt or derivative thereof, or an enantiomer or diastereomer thereof, wherein, as valence and stability permit, R¹, R², R³, R^{a}, m, n, and o are as defined above.

In some embodiments, the compound of Formula (VIII) or Formula (IX) is a compound of Formula (X):

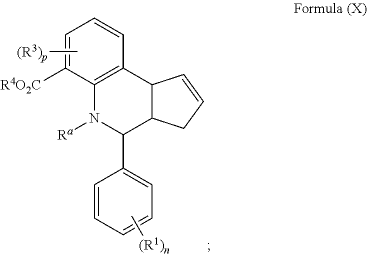

or a comestibly or biologically acceptable salt or derivative thereof, or an enantiomer or diastereomer thereof,
wherein, as a valence and stability permit:
R¹, R³, R⁴, R^{a}, and n are as defined above; and
p is 0-2.

In some embodiments, p is 0. In other embodiments, p is 1 and R³ is halo, such as bromo.

In certain embodiments, the compound of Formula (VIII) is:

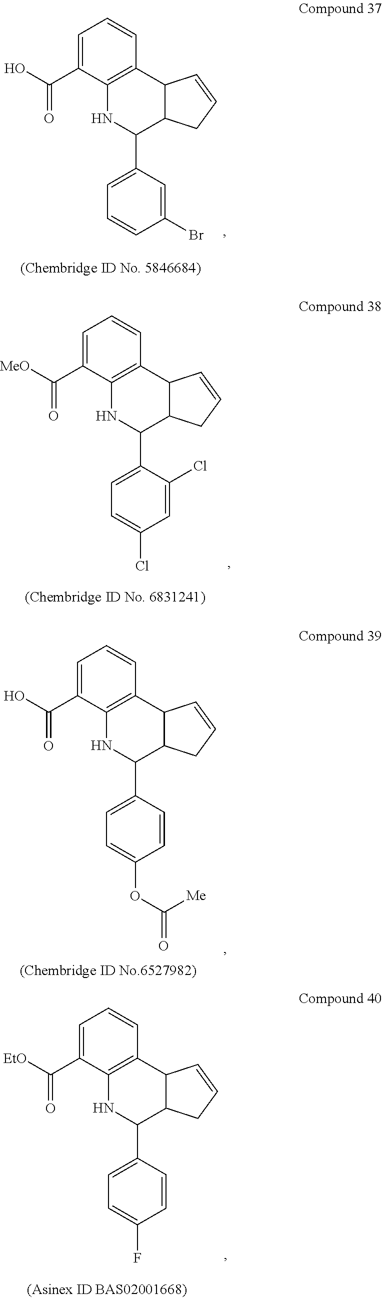 [SMILES: O=C(O)c1cccc2c1NC(c1cccc(Br)c1)C1CC=CC21 ; COC(=O)c1cccc2c1NC(c1ccc(Cl)cc1Cl)C1CC=CC21 ; CC(=O)Oc1ccc(C2Nc3c(C(=O)O)cccc3C3C=CCC32)cc1 ; CCOC(=O)c1cccc2c1NC(c1ccc(F)cc1)C1CC=CC21]

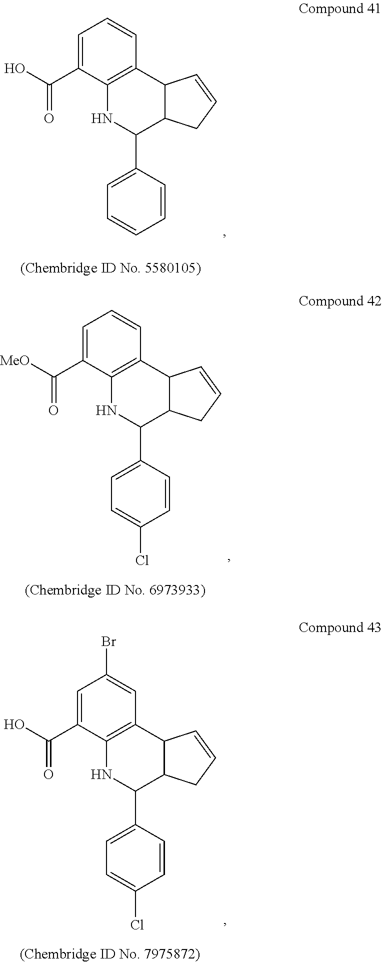 [SMILES: O=C(O)c1cccc2c1NC(c1ccccc1)C1CC=CC21 ; COC(=O)c1cccc2c1NC(c1ccc(Cl)cc1)C1CC=CC21 ; O=C(O)c1cc(Br)cc2c1NC(c1ccc(Cl)cc1)C1CC=CC21]

or a comestibly or biologically acceptable salt or derivative thereof, or an enantiomer or diastereomer thereof.

### Edible Composition Comprising Quinoline Compounds

The substituent definitions in this section (i.e., R¹, R², R³, R⁴, R⁵, Het, Ar, m and n) refer to compounds of Formula (XI). Formula (XIIa), Formula (XIIb), Formula (XIIIa), and Formula (XIIIb).

All stereochemical forms of the compounds disclosed in this and any section herein are specifically contemplated, including geometric isomers (i.e., E, Z) and optical isomers (i.e., R, S), Single stereochemical isomers as well as enantiomeric and diastereomeric mixtures of the compounds disclosed in this and any section herein are also specifically contemplated.

In some embodiments, the present invention provides a composition for reducing bitter taste of a bitter tastant, wherein the composition comprises a quinoline compound. The quinoline compounds of this invention are capable of reducing or eliminating bitter taste of a bitter tastant. In some embodiments, the composition is an edible composition. In some embodiments, the quinoline compound has a molecular weight less than about 1000, 500, or 300 daltons. In certain embodiments, the quinoline compound is a compound of Formula (XI):

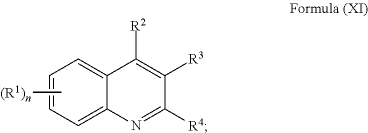

or a comestibly or biologically acceptable salt or derivative thereof, or an enantiomer or diastereomer thereof,
wherein, as valence and stability permit:
R¹, independently for each occurrence, is selected from the group consisting of C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phenyl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
R² is selected from the group consisting of hydrogen, C₁₋₁₀alkyl, Het-C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phenyl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
R³ is selected from the group consisting of hydrogen, C₁₋₁₀alkyl, Het-C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phenyl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
R⁴ is selected from the group consisting of hydrogen, C₁₋₁₀alkyl, Het-C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phenyl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₀amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
or R³ and R⁴ together with the atoms to which they are attached form a 5 to 6-membered aryl or heteroaryl ring optionally substituted by 1 to 4 groups selected from the group consisting of Het, C₁₋₁₀alkyl, C₁₋₁₀haloalkyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇-carbocyclyl, C₃₋₇carbocyclyl-C₁₋₆alkyl, C₁₋₆heterocyclyl, C₁₋₆heterocycyl-C₁₋₆alkyl, phenyl, phenyl-C₁₋₆alkyl, C₁₋₅heteroaryl, and C₁₋₅heteroaryl-C₁₋₆alkyl; and wherein heterocyclic or heteroaromatic rings, independently for each occurrence, comprise 1-4 heteroatoms selected from N, O, and S;
Het is a C₁₋₉heterocyclyl including 1-4 heteroatoms in the ring selected from oxygen, sulfur, and nitrogen;
wherein any of R¹, R², R³, R⁴, and Het, independently and independently for each occurrence, is optionally substituted with 1-3 substituents selected from the group consisting of C₁₋₁₀alkyl, C₁₋₁₀haloalkyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl sulfonamido, sulfonyl, C₃₋₇carbocyclyl, C₃₋₇carbocyclyl-C₁₋₆alkyl, C₁₋₁₀heterocyclyl, C₁₋₆heterocyclyl-C₁₋₆alkyl, C₆₋₁₀aryl, C₁₋₆alkyl-C₆₋₁₀aryl, C₆₋₁₀aryl-C₁₋₆alkyl, C₁₋₅heteroaryl, and C₁₋₅heteroaryl-C₁₋₆alkyl; and wherein heterocyclic or heteroaromatic rings, independently for each occurrence, comprise 1-4 heteroatoms selected from N, O, and S; and
n is 0-4.

According to some embodiments of compounds of Formula XI,
as valence and stability permit:
R¹, independently for each occurrence, is selected from the group consisting of halo, hydroxyl, C₁₋₆alkyl, C₁₋₆haloalkyl, C₂₋₆alkenyl, C₂₋₆alkynyl, and C₁₋₆alkoxy;
R² is selected from the group consisting of hydrogen, halo, hydroxyl, C₁₋₆alkyl, C₁₋₆haloalkyl, Het-C₁₋₆alkyl, C₂₋₆alkenyl, C₂₋₆alkynyl, and C₁₋₆alkoxy;
R³ is selected from the group consisting of hydrogen, halo, hydroxyl, C₁₋₆alkyl, C₁₋₆haloalkyl, Het-C₁₋₆alkyl, C₂₋₆alkenyl, C₂₋₆alkynyl, and C₁₋₆alkoxy;
R⁴ is selected from the group consisting of hydrogen, halo, hydroxyl, C₁₋₆alkyl, C₁₋₆haloalkyl, Het-C₁₋₆alkyl, C₂₋₆alkenyl, C₂₋₆alkynyl, and C₁₋₆alkoxy;
or R³ and R⁴ together with the atoms to which they are attached form a 5 to 6-membered aryl ring optionally substituted by 1 to 4 groups selected from the group consisting of halo, hydroxyl, C₁₋₆alkyl, C₁₋₆haloalkyl, C₂₋₆alkenyl, C₂₋₆alkynyl, C₁₋₆alkoxy, and Het;
Het is a C₂₋₆heterocyclyl including 1-3 heteroatoms in the ring selected from oxygen, sulfur, and nitrogen and is optionally substituted with one or more groups selected from the group consisting of halo, hydroxyl, C₁₋₆alkyl, C₁₋₆haloalkyl, C₂₋₆alkenyl, C₂₋₆alkynyl, C₁₋₆alkoxy, and C₆₋₁₀aryl optionally substituted by C₁₋₆alkyl;
wherein any of R¹, R², R³, R⁴, and Het, independently and independently for each occurrence, is optionally further substituted as noted above; and
n is 0-4.

According to some embodiments, one, two, or all of R², R³, or R⁴ is not hydrogen.

According to some embodiments of the compound of Formula (XI), R³ and R⁴ together with the atoms to which they are attached form a 5 to 6-membered aryl ring, such as a benzo ring, optionally substituted as described above. For example, in certain embodiments, the compound of Formula (XI) is a compound of Formula (XIIa):

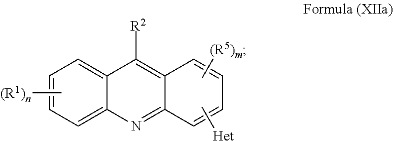

or a comestibly or biologically acceptable salt or derivative thereof, or an enantiomer or diastereomer thereof,
wherein, as valence and stability permit:
R¹, R², Het, and n are as defined above;
R⁵, independently for each occurrence, is selected from the group consisting of halo, hydroxyl, C₁₋₆alkyl, C₁₋₆haloalkyl, C₂₋₆alkenyl, C₂₋₆alkynyl, and C₁₋₆alkoxy; and
m is 0-3.

According to some embodiments, Het is a nitrogen-containing heterocycle optionally including additional heteroatoms selected from oxygen, sulfur, and nitrogen and optionally substituted as described above. For example, in certain embodiments, the compound of Formula (XIIa) is a compound of Formula (XIIIa):

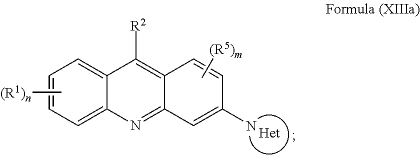

or a comestibly or biologically acceptable salt or derivative thereof, or an enantiomer or diastereomer thereof, wherein, as valence and stability permit, R¹, R², R⁵, Het, n, and m are as defined above.

In certain embodiments, one or more occurrences of R⁵ is halo, such as fluoro. For example, in some embodiments, for compounds of Formula (XIIa) and Formula (XIIIa), m is 3 and R⁵ is fluoro for each occurrence.

In some embodiments. Het is a nitrogen-containing heterocycle, such as aziridine, azetidine, diazetidine, pyrrolidine, pyrroline, imidazolidine, pyrazolidine, imidazoline, oxazolidine, isoxazolidines, oxazoline, piperidine, piperazine, morpholine, oxazine, thiazine, azepane, azepine, or diazepine optionally substituted as described above. In particular embodiments, Het is pyrrolidine, piperazine, or morpholine optionally substituted as described above. In certain embodiments, Het is substituted with one or more C₁₋₆alkyl, such as methyl.

According to certain embodiments, n is 0 or n is 1 and R¹ is C₁₋₆alkyl, such as methyl; R² is C₁₋₆alkyl, such as methyl; m is 3 and R⁵ is fluoro for each occurrence; and Het is pyrrolidine, piperazine, or morpholine optionally substituted with one or more C₁₋₆alkyl, such as methyl. For instance, in certain embodiments, the compound of Formula (XI), Formula (XIIa), or Formula (XIIIa) is:

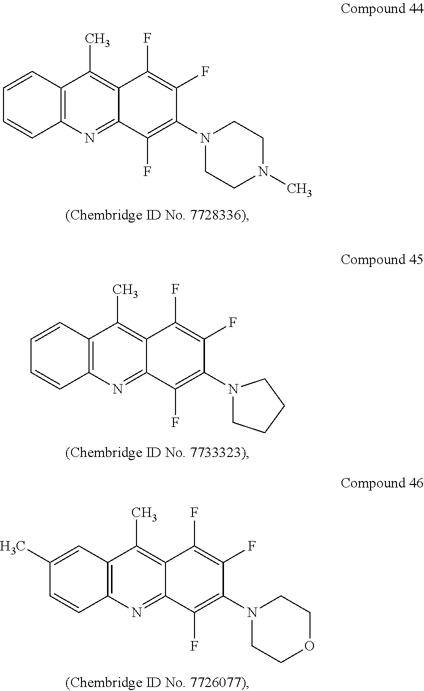

or a comestibly or biologically acceptable salt or derivative thereof, or an enantiomer or diastereomer thereof.

According to some embodiments of the compound of Formula (XI), R³ and R⁴, independently, are selected from the group consisting of hydrogen, halo, hydroxyl, C₁₋₆alkyl, C₁₋₆haloalkyl, Het-C₁₋₆alkyl C₂₋₆alkenyl, C₂₋₆alkynyl, and C₁₋₆alkoxy. For example, in certain embodiments, R³ is C₁₋₆alkyl, such as methyl. In some embodiments, R⁴ is Het-C₁₋₆alkyl, such as Het-CH₂—. In further embodiments, R³ is C₁₋₆alkyl, such as methyl, and R⁴ is Het-C₁₋₆alkyl, such as Het-CH₂—. For example, in certain embodiments, the compound of Formula (XI) is a compound of Formula (XIIb):

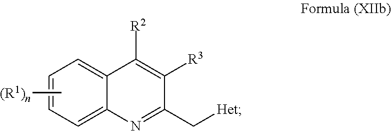

or a comestibly or biologically acceptable salt or derivative thereof, or an enantiomer or diastereomer thereof, wherein, as valence and stability permit, R¹, R², R³, Het, and n are as defined above.

According to some embodiments, Het is a nitrogen-containing heterocycle optionally including additional heteroatoms selected from oxygen, sulfur, and nitrogen and optionally substituted as described above. In certain embodiments, Het is substituted by one or more C₆₋₁₀aryl, such as phenyl or naphthyl, optionally substituted by C₁₋₆alkyl. For example, in certain embodiments, the compound of Formula (XIIb) is a compound of Formula (XIIb):

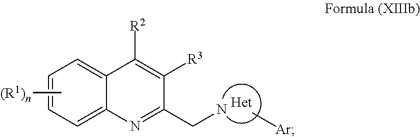

or a comestibly or biologically acceptable salt or derivative thereof, or an enantiomer or diastereomer thereof,
wherein, as valence and stability permit:
R¹, R², R³, Het, and n are as defined above; and
Ar is C₆₋₁₀aryl, such as phenyl or naphthyl, optionally substituted by C₁₋₆alkyl.

In some embodiments, Het is a nitrogen-containing heterocycle, such as aziridine, azetidine, diazetidine, pyrrolidine, pyrroline, imidazolidine, pyrazolidine, imidazoline, oxazolidine, isoxazolidines, oxazoline, piperidine, piperazine, morpholine, oxazine, thiazine, azepane, azepine, or diazepine optionally substituted as described above. In specific embodiments, Het is pyrrolidine, piperazine, or morpholine, particularly piperazine, optionally substituted as described above.

According to certain embodiments, one or more occurrences of R¹ is C₁₋₆alkyl, such as methyl; R² is hydroxyl; R³ is C₁₋₆alkyl, such as methyl; Het is piperazine; and Ar is phenyl. For instance, in some embodiments, the compound of Formula (XI), Formula (XIIb), or Formula (XIIIb) is:

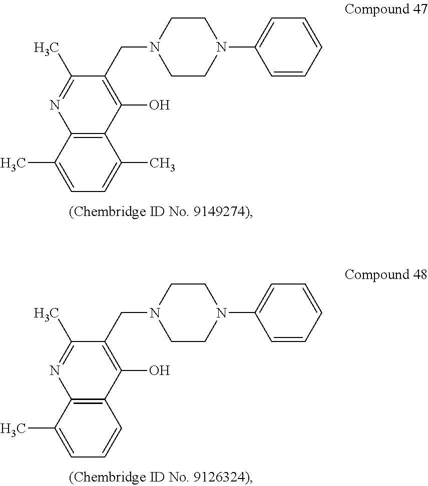 [SMILES: Cc1nc2c(C)ccc(C)c2c(O)c1CN1CCN(c2ccccc2)CC1 ; Cc1nc2c(C)cccc2c(O)c1CN1CCN(c2ccccc2)CC1]

or a comestibly or biologically acceptable salt or derivative thereof, or an enantiomer or diastereomer thereof.

In some embodiments of the above compounds, n is 0, and there are no occurrences of R¹. In other occurrences, n is not zero. For example, in some embodiments, one or more occurrences of R¹ are C₁₋₆alkyl, such as methyl. For instance, in certain embodiments, n is 1 and R¹ is C₁₋₆alkyl, such as methyl, optionally in a position para to the nitrogen atom. In another embodiment, n is 2 and R¹ is C₁₋₆alkyl, such as methyl, for both occurrences, with the occurrences of R¹ optionally in a 1,4-relationship.

In some embodiments of the above compounds, R² is hydrogen. In other embodiments, R² is hydroxyl. In other embodiments, R² is C₁₋₆alkyl, such as methyl.

### Edible Compositions Comprising N-Phenylalkylamide Compounds

The substituent definitions in this section (i.e., R¹, R², R^{a}, Ar, X, m, n and p) refer to compounds of Formula (XIV), Formula (XVa), Formula (XVb), and Formula (XVc).

All stereochemical forms of the compounds disclosed in this and any section herein are specifically contemplated, including geometric isomers (i.e., E, Z) and optical isomers (i.e., R, S). Single stereochemical isomers as well as enantiomeric and diastereomeric mixtures of the compounds disclosed in this and any section herein are also specifically contemplated.

In some embodiments, the present invention provides a composition for reducing bitter taste of a bitter tastant, wherein the composition comprises a N-phenylalkylamide compound. The N-phenylalkylamide compounds of this invention are capable of reducing or eliminating bitter taste of a bitter tastant. In some embodiments, the composition is an edible composition. In some embodiments, the N-phenylalkylamide compound has a molecular weight less than about 1000, 500, or 300 daltons. In certain embodiments, the N-phenylalkylamide compound is a compound of Formula (XIV):

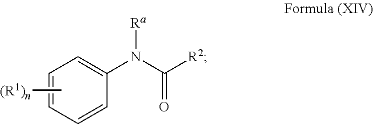

or a comestibly or biologically acceptable salt or derivative thereof, or an enantiomer or diastereomer thereof,
wherein, as valence and stability permit:
R¹, independently for each occurrence, is selected from the group consisting of C₁₋₁₀alkyl, C₁₋₁₀haloalkyl, C₂₋₁₀alkenyl, C₂₋₁₀alkynyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phenyl-C₁₋₆alkyloxy, C₁₋₅heteroaryloxy, C₁₋₅heteroaryl-C₁₋₆alkyloxy, C₃₋₁₀alkenyloxy, C₃₋₁₀alkynyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
R² is selected from the group consisting of C₁₋₁₀alkyl, C₁₋₁₀haloalkyl, C₂₋₁₀alkenyl, C₂₋₁₀alkynyl, hydroxyl, C₁₋₁₀alkoxy, C₆₋₁₀aryloxy, C₆₋₁₀aryloxy-C₁₋₁₀alkyl, C₆₋₁₀arylamino-C₁₋₁₀alkyl, C₆₋₁₀aryl-C₁₋₆alkyloxy, C₁₋₁₀heteroaryloxy, C₁₋₉heteroaryloxy-C₁₋₆alkyl, C₁₋₉heteroarylamino-C₁₋₆alkyl, C₁₋₉heteroaryl-C₁₋₆alkyloxy, C₃₋₁₀alkenyloxy, C₃₋₁₀alkynyloxy, amino, diC₁₋₁₀alkylamino, monoC₁₋₁₀alkylamino, sulfhydryl, C₁₋₁₀alkylthio, C₃₋₁₀carbocyclyl, C₃₋₁₀carbocyclyloxy, C₃₋₁₀carbocyclyl-C₁₋₆alkyl, C₃₋₁₀carbocyclyloxy-C₁₋₆alkyl, C₃₋₁₀carbocyclylamino-C₁₋₆alkyl, C₁₋₉heterocyclyl, C₁₋₉heterocyclyl-C₁₋₆alkyl, C₁₋₉heterocyclyloxy-C₁₋₆alkyl, C₁₋₉heterocyclylamino-C₁₋₆alkyl, C₆₋₁₀aryl, C₆₋₁₀aryl-C₁₋₆alkyl, C₁₋₉heteroaryl, and C₁₋₉heteroaryl-C₁₋₆alkyl, wherein heterocyclic or heteroaromatic rings, independently for each occurrence, comprise 1-4 heteroatoms selected from N, O, and S;
R^{a} is selected from the group consisting of hydrogen, C₁₋₁₀alkyl, C₁₋₁₀haloalkyl, C₂₋₁₀alkenyl, C₂₋₁₀alkynyl, carboxyl, C₁₋₁₀alkoxycarbonyl, C₂₋₁₀alkenyloxycarbonyl, C₂₋₁₀alkynyloxycarbonyl, C₁₋₁₀acyl, phosphoryl, phosphonate, phosphinate, cyano, sulfonate, sulfamoyl, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl, wherein heterocyclic or heteroaromatic rings, independently for each occurrence, comprise 1-4 heteroatoms selected from N, O, and S;
wherein any of R¹, R², and R^{a}, independently and independently for each occurrence, is optionally substituted with 1-3 substituents selected from the group consisting of C₁₋₁₀alkyl, C₁₋₁₀haloalkyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, C₆₋₁₀aryloxy, C₆₋₁₀arylamino, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl; and wherein heterocyclic or heteroaromatic rings, independently for each occurrence, comprise 1-4 heteroatoms selected from N, O, and S; and
n is 0-3.

According to some embodiments of compounds of Formula (XIV),
as valence and stability permit:
R¹, independently for each occurrence, is selected from the group consisting of halo, hydroxyl, C₁₋₆alkyl, C₁₋₆haloalkyl, C₂₋₆alkenyl, C₂₋₆alkynyl, C₁₋₆alkoxy, and C₁₋₆acyloxy;
R² is selected from the group consisting of C₁₋₆alkyl, C₁₋₆alkoxy-substituted C₁₋₆alkyl, C₆₋₁₀aryloxy-substituted C₁₋₆alkyl, C₂₋₆alkenyl, C₂₋₆alkynyl, C₆₋₁₀aryl-C₁₋₆alkyl, and —((CH₂)ₘX)ₚ—Ar, wherein aryl groups of R² are optionally substituted by one or more halo, hydroxyl, C₁₋₆alkyl, C₁₋₆haloalkyl, C₁₋₆alkoxy, or C₁₋₆acyloxy;
R^{a} is selected from the group consisting of hydrogen, C₁₋₆alkyl, C₂₋₆alkenyl, and C₂₋₆alkynyl;
X is selected from the group consisting of O, NH, and CH₂;
Ar is selected from the group consisting of C₆₋₁₀aryl, C₄₋₉heteroaryl, C5-10carbocyclyl, and C₄₋₉heterocyclyl, including fused bicyclic groups, wherein Ar is optionally substituted by one or more halo, hydroxyl, C₁₋₆alkyl, C₁₋₆haloalkyl, C₁₋₆alkoxy, or C₁₋₆acyloxy;
wherein any of R¹, R², and R^{a}, independently and independently for each occurrence, is optionally further substituted as noted above;
m is 1-3;
n is 0-3: and
S is 0 or 1.

In certain embodiments, n is 0. In other embodiments, n is 1-3. For example, in some embodiments, n is 1-3, such as 2, and one or more occurrences of R¹ is C₁₋₆alkoxy, such as methoxy. In some embodiments, n is 1-3, such as 2, and one or more occurrences of R¹ is C₁₋₆alkoxy, such as methoxy, and one or more occurrences of R¹ is halo, such as chloro. In some embodiments, n is 1-3, such as 2, and one or more occurrences of R¹ is C₁₋₆alkyl, such as methyl or ethyl, and one or more occurrences of R¹ is halo, such as chloro. In further embodiments, n is 2-3, such as 2, and two or more occurrences of R¹ is C₁₋₆alkyl, such as methyl or ethyl.

In some embodiments, R² is C₁₋₆alkyl, such as methyl, ethyl, or propyl. In certain embodiments, R² is C₆₋₁₀aryloxy-substituted C₁₋₆ alkyl, such as 2-aryloxymethyl (e.g., 2-phenyloxyethyl), optionally substituted as described above. In some embodiments, R² is C₆₋₁₀aryl-C₁₋₆alkyl. For example, in some embodiments, R² is phenyl-C₁₋₆alkyl, such as 2-arylethyl (e.g., dihydrocinnamyl) or 3-arylpropyl (e.g., 3-phenylpropyl), optionally substituted as described above.

According to certain embodiments, R^{a} is hydrogen. In other embodiments, R^{a} is C₁₋₆alkyl, such as methyl.

As noted above, in certain embodiments, R² is C₁₋₆alkyl. For instance, in some embodiments, the compound of Formula (XIV) is a compound of Formula (XVa):

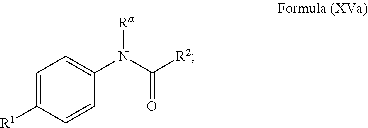

or a comestibly or biologically acceptable salt or derivative thereof, or an enantiomer or diastereomer thereof;
wherein, as valence and stability permit:
R¹ and R^{a} are as defined above; and
R² is C₁₋₆alkyl, such as methyl or ethyl.

According to certain embodiments of the compound of Formula (XIV), R² is —((CH₂)ₘX)ₚ—Ar. In certain embodiments, p is 0. In other embodiments, p is 1. For example, in some embodiments, p is 1 and X is O. In some embodiments, p is 1 and X is CH₂. In some embodiments, Ar is C₆₋₁₀aryl, such as phenyl, optionally substituted as described above. In other embodiments, Ar is C₄₋₉heterocyclyl (e.g. dioxane), including fused bicyclic groups, such as benzo-fused heterocyclyl (e.g., benzo-fused dioxane), optionally substituted as described above.

As noted above, according to some embodiments, p is 1. For example, in some embodiments, the compound of Formula (XIV) is a compound of Formula (XVb):

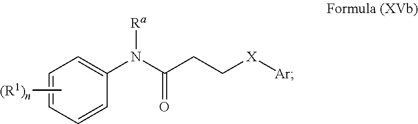

or a comestibly or biologically acceptable salt or derivative thereof, or an enantiomer or diastereomer thereof wherein, as valence and stability permit, R¹, R^{a}, X, Ar, and n are as defined above.

As noted above, according to some embodiment, p is 0. For example, in some embodiments, the compound of Formula (XIV) is a compound of Formula (XVc):

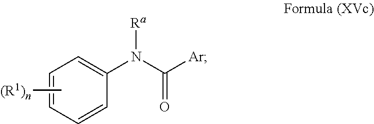

or a comestibly or biologically acceptable salt or derivative thereof, or an enantiomer or diastereomer thereof, wherein, as valence and stability permit, R¹, R^{a}, Ar, and n are as defined above.

In certain embodiments, the compound of formula (XIV) is:

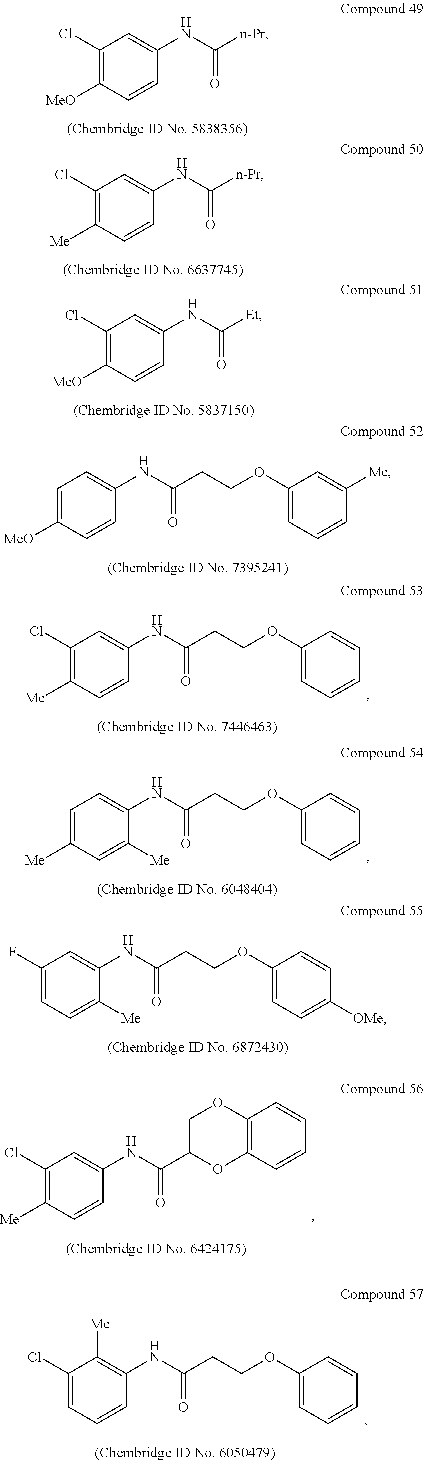

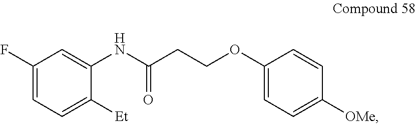 [SMILES: CCc1ccc(F)cc1NC(=O)CCOc1ccc(OC)cc1]

or a comestibly or biologically acceptable salt or derivative thereof, or an enantiomer or diastereomer thereof.

In some embodiments, the edible composition of this invention comprise diphenyl-containing compounds, pyrazole-containing compounds, hydroquinoline compounds, quinoline compounds, or N-phenylalkylamide compounds as described herein, or a comestibly or biologically acceptable salt or derivative thereof, or an enantiomer or diastereomer thereof, or mixtures thereof.

if a comestibly or biologically acceptable salt of a compound of the present invention is used, such salt is preferably derived from inorganic or organic acids and bases. Examples of such salts include, but are not limited to, acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate, Salts derived from appropriate bases include alkali metal (e.g., sodium and potassium), alkaline earth metal (e.g., magnesium), ammonium and N⁺(C₁₋₄alkyl)₄ salts. This invention also envisions the quaternization of any basic nitrogen-containing groups of the compounds disclosed herein. Water or oil-soluble or dispersible products may be obtained by such quaternization. In some embodiments, the compounds of the present invention are present as sodium, potassium or citrate salts.

Another aspect of the present invention provides edible compositions comprising a) a compound of the invention; and b) a bitter tastant. In some embodiments, the compound of the invention is a compound having a molecular weight less than about 1000, 500, or 300 daltons. In certain embodiments, the compound of the invention is a compound of Formula (I), Formula (IIa), Formula (IIb), Formula (IIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIa), Formula (XIIIb), Formula (XIV), Formula (XVa), Formula (XVb) or Formula (XVc), as described herein, or combinations thereof. In some embodiments, the compound of the invention is a compound selected from Compounds 1-58 or combinations thereof.

In some embodiments, the bitter tastant present in the edible composition is a bitter tasting salt. In some embodiments, the bitter tastant present in the edible composition is a potassium salt, a magnesium salt, or a calcium salt. In some embodiments, the bitter tastant present in the edible composition is a potassium salt. In some embodiments, the bitter tastant present in the edible compositions is KCl. In other embodiments, the bitter tastant present in the edible composition is potassium lactate.

In another embodiment, the edible compositions comprise a) a compound of the invention; and b) a potassium salt. In some embodiments, the potassium salt is KCl or potassium lactate. In specific embodiments, the potassium salt is KCl. In certain embodiments, the compound of the invention is a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof. In some embodiments, the compound of the invention is a compound selected from Compounds 1-58 or combinations thereof.

In some embodiments, the edible composition furthers comprise a sodium salt. In some embodiments, the edible compositions further comprise NaCl. In some embodiments, the edible compositions further comprise sodium lactate. In some embodiments, the edible compositions further comprise sugar.

In some embodiments, the edible composition further comprises one or more additional components selected from the group consisting of preservatives, nutritives, flavorants or additional flavor modifiers, which may lack an inherent flavor.

In some embodiments, the edible composition further comprises one or more emulsifiers. Sodium and potassium based emulsifiers are commonly used as emulsifiers in the food art. Sodium-based emulsifiers include, e.g., sodium salts of fatty acids, sodium alginate, sodium aluminum phosphate, sodium caseinate, sodium metaphosphate, sodium phosphate (dibasic), sodium phosphate (monobasic), sodium phosphate (tribasic), sodium polyphosphate, sodium pyrophosphate, and sodium stearoyl lactylate. Potassium-based emulsifiers include, e.g., potassium salts of fatty acids, potassium alginate, potassium citrate, potassium phosphate (dibasic), potassium phosphate (monobasic), potassium phosphate (tribasic), potassium polyphosphate, potassium polymetaphosphate, and potassium pyrophosphate. Accordingly, some embodiments of the present invention include replacing a sodium-based emulsifier with a potassium based emulsifier and adding a compound of the present invention.

In some embodiments, the edible composition further comprises a surfactant to increase or decrease the effectiveness of the compounds of the present invention. Suitable surfactants include, but are not limited to, non-ionic surfactants (e.g., mono and diglycerides, fatty acid esters, sorbitan esters, propylene glycol esters, and lactylate esters) anionic surfactants (e.g., sulfosuccinates and lecithin) and cationic surfactants (e.g., quaternary ammonium salts).

In some embodiments wherein the edible compositions further comprises a preservative, the preservative improves the shelf life of the edible composition. Suitable preservatives include, but are not limited to ascorbic acid, benzoic acid, butyl p-hydroxybenzoate, calcium benzoate, calcium disodium EDTA, calcium hydrogen sulfite, calcium propionate, calcium sorbate, chitosan, cupric sulfate, dehydroacetic acid, diethyl percarbonate, dimethyl dicarbonate, disodium EDTA, E-polylysine glycine, erythorbic acid, ethyl p-hydroxybenzoate, formic acid, gum guaiac, heptylparaben, hinokitiol, isobutyl paraoxybenzoate, Japanese styrax benzoin extract, methylparaben, milt protein extract, natanycin, nisin, peptin extract, 2-phenylphenol, pimaricin, potassium acetate, potassium benzoate, potassium lactate, potassium metabisulfite, potassium nitrate, potassium nitrite, potassium pyrosulfite, potassium sorbate, potassium sulfite, propionic acid, propyl p-hydroxybenzoate, propyl p-oxybenzoate, propylene oxide, propylparaben, sodium benzoate, sodium bisulfate, sodium dehydroacetate, sodium diacetate, sodium erythorbate, sodium hydrogen sulfite, sodium hypophosphite, sodium hyposulfite, sodium metabisulfite, sodium nitrate, sodium nitrite, sodium o-phenylphenol, sodium propionate, sodium pyrosulfite, sodium sulfite, sodium thiocyanate, sorbic acid and sulfur dioxide. In some embodiments, the preservative has a bitter flavor.

In some embodiments, the composition may further comprise one or more additional components selected from the group consisting of flow agents, processing agents, sugars, amino acids, other nucleotides, and sodium or potassium salts of organic acids such as citrate and tartarate. Such additional ingredients may add flavor, or aid in blending, processing or flow properties of the edible composition.

In some embodiments, the rate of release of the compound of the present invention is regulated. The release rate of the compound of the present invention can be altered by, for example, varying its solubility in water. Rapid release can be achieved by encapsulating the compound of the present invention with a material with high water solubility. Delayed release of the compound of the present invention can be achieved by encapsulating the compound of the present invention with a material with low water solubility. The compound of the present invention can be co-encapsulated with carbohydrates or masking tastants such as sweeteners. The rate of release of the compound of the present invention can also be regulated by the degree of encapsulation. In some embodiments, the compound of the present invention is fully encapsulated. In other embodiments, the compounds of the present invention are partially encapsulated. In some embodiments, the rate of release is regulated so as to release with the bitter tastant.

The edible compositions of this invention are prepared according to techniques well-known in the art. In general, an edible composition of the invention is prepared by mixing a component or ingredient of the edible composition with a compound of the invention. Alternatively, a compound of the invention can be added directly to the edible composition. In some embodiments, a bitter tastant is added simultaneously or sequentially with a compound of the invention. If sequentially, the bitter tastant may be added before or after the compound of the invention. In some embodiments, the edible composition is a food product. In some embodiments, the edible composition is a pharmaceutical composition. In some embodiments, the edible composition is a consumer product.

The amount of both a compound of the present invention and a bitter tastant used in an edible composition depends upon a variety of factors, including the desired or acceptable perception of bitterness, saltiness, or sweetness. The amount may depend on the nature of the edible composition, the particular compound added, the bitter tastant, other compounds present in the composition, the method of preparation (including amount of heat used), and the pH of the edible composition. It will be understood that those of skill in the art will know how to determine the amounts needed to produce the desired taste(s).

In general, a compound of the present invention in an edible composition may be present at a concentration between about 0.001 ppm and 1000 ppm. In some embodiments, the edible composition comprises between about 0.005 to 500 ppm; 0.01 to 100 ppm; 0.05 to 50 ppm; 0.1 to 5 ppm; 0.1 to 10 ppm; 1 to 10 ppm; 1 to 30 ppm; 1 to 50 ppm; 10 to 30 ppm; 10 to 50 ppm; or 30 to 50 ppm of a compound of the present invention. In yet other embodiments, the edible composition comprises about 0.1 to 30 ppm, 1 to 30 ppm or 1 to 50 ppm of a compound of the present invention. In additional embodiments, the edible composition comprises about 0.1 to 5 ppm; 0.1 to 4 ppm; 0.1 to 3 ppm; 0.1 to 2 ppm; 0.1 to 1 ppm; 0.5 to 5 ppm; 0.5 to 4 ppm; 0.5 to 3 ppm; 0.5 to 2 ppm; 0.5 to 1.5 ppm; 0.5 to 1 ppm; 5 to 15 ppm; 6 to 14 ppm; 7 to 13 ppm; 8 to 12 ppm; 9 to 11 ppm; 25 to 35 ppm; 26 to 34 ppm; 27 to 33 ppm; 28 to 32 ppm; or 29 to 31 ppm.

In yet other embodiments, the edible composition comprises about 0.1 ppm, about 0.5 ppm, about 1 ppm, about 2 ppm, about 0.3 ppm, about 4 ppm, about 5 ppm, about 6 ppm, about 7 ppm, about 8 ppm, about 9 ppm, or about 10 ppm of a compound of the present invention. In other embodiments, the edible composition comprises about 11 ppm, about 12 ppm, about 13 ppm, about 14 ppm, about 15 ppm, about 16 ppm, about 17 ppm, about 18 ppm, about 19 ppm, about 20 ppm, about 21 ppm, about 22 ppm, about 23 ppm, about 24 ppm, about 25 ppm, about 26 ppm, about 27 ppm, about 28 ppm about, 29 ppm, or about 30 ppm of a compound of the present invention.

In still other embodiments, the edible composition comprises about 31 ppm, about 32 ppm, about 33 ppm, about 34 ppm, about 35 ppm, about 36 ppm, about 37 ppm, about 38 ppm, about 39 ppm, about 40 ppm, about 41 ppm, about 42 ppm, about 43 ppm, about 44 ppm, about 45 ppm, about 46 ppm, about 47 ppm, about 48 ppm, about 49 ppm, or about 50 ppm of a compound of the present invention.

In other embodiments, the edible composition comprises more than about 0.5 ppm, 1 ppm, 5 ppm, 10 ppm, 15 ppm, 20 ppm, 25 ppm, or 30 ppm of a compound of the present invention, up to, for example, about 30 ppm or 50 ppm. In additional embodiments, the edible composition comprises less than about 50 ppm, 30 ppm, 25 ppm, 20 ppm, 15 ppm, 10 ppm, 5 ppm, 1 ppm, or 0.5 ppm of a compound of the present invention. In yet additional embodiments, the edible composition comprises less than about 30 ppm, 10 ppm, or 1 ppm of a compound of the present invention.

When the edible composition comprises KCl, the amount of KCl will vary depending on the nature of the edible composition, the amount of perceived saltiness desired and the presence of other compounds in the composition. In some embodiments, KCl is present at a concentration between about 0.001-5% w/w; 0.01-5% w/w; 0.1-5% w/w; 5-4.8% w/w; 5-4% w/w; 0.5-3% w/w; 0.75-3% w/w; 1-2.5% w/w; or 1-2% w/w. In some embodiments, KCl is present at a concentration of about 0.5% w/w, about 1% w/w, about 1.5% w/w, about 2% w/w, about 2.5% w/w, about 3% w/w, about 3.5% w/w, about 4% w/w, about 4.5% w/w, or about 5% w/w. In some embodiments, KCl is present at a concentration of up to about 0.5% w/w, up to about 1% w/w, up to about 1.5% w/w, up to about 2% w/w, up to about 2.5% w/w, up to about 3% w/w, up to about 3.5% w/w, up to about 4% w/w, up to about 4.5% w/w, or up to about 5% w/w. In some embodiments, KCl is present at a concentration of about 2% w/w.

In some embodiments, KCl is added to the edible composition as a salt substitute in an amount sufficient to replace NaCl. For example, the amount of KCl in the edible composition may range from about 0.5 to about 1.5 times the replaced NaCl depending upon the application, e.g., if about 0.5 mg of NaCl is replaced, about 0.25 to about 0.75 mg of KCl is added. Typically, KCl is added in the same weight amount as the NaCl being replaced.

Similarly, when the edible composition comprises potassium lactate, the amount of potassium lactate added varies depending on the nature of the edible composition, the amount of preservation required and the presence of other compounds in the composition. Potassium lactate may be present at a concentration between about 0.001-5% w/w; 0.01-5% w/w; 0.1-5% w/w; 0.5-4.8% w/w; 0.5-4% w/w; 0.5-3% w/w; 0.75-3% w/w; 1-2.5% w/w; or 1-2% w/w.

In some embodiments, potassium lactate is added to the edible composition in an amount sufficient to replace sodium lactate. For example, the amount of potassium lactate in the food or beverage after the sodium lactate substitute is added may range from about 0.5 to about 1.5 limes the replaced sodium lactate depending upon the application, e.g., if about 0.5 mg of sodium lactate is replaced, about 0.25 to about 0.75 mg of potassium lactate is added. Typically, potassium lactate will be added in the same weight amount as the sodium lactate being replaced.

Further, when the edible composition comprises an artificial sweetener, such as Acesulfame K, the amount of the sweetener added varies depending on the nature of the edible composition, the amount of sweetness required and the presence of other compounds in the composition. Acesulfame K, for example, may be present at a concentration between about 1-200 ppm, 10-200 ppm, 50-150 ppm, 50-125 ppm, 75-125 ppm, and 75-100 ppm, preferably about 75 ppm.

In some embodiments, an artificial sweetener is added to the edible composition in an amount sufficient to replace sugar. In some embodiments, the artificial sweetener has a bitter taste or aftertaste. In some embodiments, the artificial sweetener is Acesulfame K. For example, the amount of Acesulfame K in the edible composition may range from about 0.001 to about 0.01 times the replaced sugar depending upon the application, e.g., if about 100 mg of sugar is replaced, about 0.1 to about 1 mg of Acesulfame K is added. Typically, Acesulfame K will be added in about 0.005 times the amount of sugar being replaced.

In some embodiments, the edible compositions are included in a package. In some embodiments, the edible composition is packaged in bulk, in which the package contains more of the compositions than would typically be used for a single dish or serving of food or beverage. Such bulk packages can be in the form of paper, plastic, or cloth bags or cardboard boxes or drums. Such bulk packages may be fitted with plastic or metal spouts to facilitate the dispensing of the edible composition.

In some embodiments, the package contains an edible composition comprising a compound of the present invention and a bitter tastant. In some embodiments, the package contains an edible composition comprising a compound of the present invention and bitter tasting salt. In some embodiments, the package contains an edible composition comprising a compound of the present invention and a potassium salt, a magnesium salt, or a calcium salt. In some embodiments, the package contains an edible composition comprising a compound of the present invention and a potassium salt. In some embodiments, the package contains an edible composition comprising a compound of the present invention and KCl. In other embodiments, the package contains an edible composition comprising a compound of the present invention and potassium lactate. In some embodiments, the package contains an edible composition comprising a compound of the present invention a potassium salt, and a sodium salt. In other embodiments, the package contains an edible composition comprising a compound of the present invention, KCl and NaCl. In yet other embodiments, the package contains an edible composition comprising a compound of the present invention, potassium lactate and sodium lactate. In other embodiments, the package contains an edible composition comprising a compound of the present invention and Acesulfame K and sugar. In other embodiments, the package contains an edible composition comprising a compound of the present invention, potassium lactate, KCl and NaCl.

In some embodiments, the edible compositions of the present invention are compositions suitable to be used as seasonings, as ingredients in food products or as condiments. In such embodiments, the edible composition may or may not contain a bitter tastant. For example, the edible composition may be used in, e.g., a seasoning which comprises a bitter tastant such as, e.g., KCl. Such seasonings can be used in the place of table salt (i.e., NaCl) to season prepared food products. Alternatively, the edible composition may be used in, e.g., a seasoning which does not contain a bitter tastant. Such seasonings can be used to season prepared food products which contain a bitter tastant (either inherently present or added during preparation) in order to reduce the bitter taste associated with the bitter tastant. In some embodiments, the edible composition is a seasoning comprising KC and a compound of the invention. In some embodiments, the edible composition is a seasoning comprising KCl, NaCl and a compound of the invention. In some embodiments the seasoning further comprises a spice or a blend of spices.

Alternatively, the edible compositions may be used for medicinal or hygienic purposes, for example, in soaps, shampoos, mouthwash, medicines, pharmaceuticals, cough syrup, nasal sprays, toothpaste, dental adhesives, tooth whiteners, glues (e.g., on stamps and envelopes), and toxins used in insect and rodent control.

### Food Product

In some embodiments, the edible composition is a food product. According to such embodiments, the food product comprises (a) a food stuff; and (b) a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described above, or combinations thereof.

In some embodiments, the food product further comprises a bitter tastant, as described herein. In some embodiments, the bitter tastant is a potassium salt, such as KCl or potassium lactate. In specific embodiments, the potassium salt is KCl.

In some embodiments, the food product further comprises one or more additional flavor modifiers.

In some embodiments, the food product further comprises one or more additional components selected from the group consisting of preservatives, nutritives, flavorants or additional flavor modifiers, which may lack an inherent flavor.

### Pharmaceutical Composition

In some embodiments, the edible composition is a pharmaceutical composition. According to such embodiments, the pharmaceutical composition comprises (a) a bitter tasting pharmaceutically active ingredient; and (b) a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described above, or combinations thereof.

According to some embodiments, the pharmaceutical composition can comprise any bitter tasting pharmaceutically active ingredient. Non-limiting examples of bitter pharmaceutical compounds include: acetaminophen, ampicillin, azithromycin, chlorpheniramine, cimetidine, dextromethorphan, diphenhydramine, erythromycin, ibuprofen, penicillin, phenylbutazone, pseudoephedrine, ranitidine, spironolactone statins (including, but not limited to, atorvastatin, cerivastatin, fluvastatin, lovastatin, mevastatin, pitavastatin, pravastatin, rosuvastatin, and simvastatin) and theophylline.

In other embodiments, the invention provides a pharmaceutical composition comprising (a) a pharmaceutically active ingredient; (b) a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described above, or combinations thereof; and (c) a bitter tastant. In such embodiments, the pharmaceutical compositions may comprise any pharmaceutically active ingredient.

In other embodiments, the invention provides a pharmaceutical composition comprising (a) a pharmaceutically active ingredient; (b) a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described above, or combinations thereof; and (c) a potassium salt. In some embodiments, the potassium salt is KCl or potassium lactate. In some embodiments, the potassium salt is KCl.

In some embodiments, the pharmaceutical composition further comprises one or more additional flavor modifiers.

In some embodiments, the pharmaceutical composition further comprises one or more additional components selected from the group consisting of preservatives, nutritives, flavorants or additional flavor modifiers, which may lack an inherent flavor.

### Consumer Product

In some embodiments, the edible compositions is a consumer product. According to such embodiments, the consumer product comprises (a) a bitter tastant and (b) a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described above, or combinations thereof.

According to another embodiment, the invention provides a consumer product comprising (a) a potassium salt; and (b) a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described above, or combinations thereof. In some embodiments, the potassium salt is KCl or potassium lactate. In some embodiments, the potassium salt is KCl.

In other embodiments, the invention provides a consumer product for reducing bitter taste of a bitter tastant, wherein said consumer product comprises a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described above, or combinations thereof. In some embodiments, the bitter tastant is a potassium salt. In some embodiments, the potassium salt is KCl or potassium lactate. In some embodiments, the bitter tastant is KCl.

In some embodiments, the consumer product further comprises one or more additional flavor modifiers.

In some embodiments, the consumer product further comprises one or more additional components selected from the group consisting of preservatives, nutritives, flavorants or additional flavor modifiers, which may lack an inherent flavor.

### Method of Preparing an Edible Composition

According to another aspect, the invention provides a method of preparing an edible composition. The method comprises: (a) providing a comestibly acceptable carrier; and (b) adding to the comestibly acceptable carrier of (a) a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described above, or combinations thereof, with the comestibly acceptable carrier. In some embodiments, the compound of the invention has been dissolved in a solvent prior to the addition step (b).

In some embodiments, the comestibly acceptable carrier in (a) is inherently bitter. In such embodiments, the comestibly acceptable carrier may inherently contain a bitter tastant. In some embodiments, the inherent bitter tastant is a bitter tasting salt. In some embodiments, the inherent bitter tastant is a potassium salt, a magnesium salt, or a calcium salt. In some embodiments, the inherent bitter tastant is a potassium salt. In some embodiments, the inherent bitter tastant is KCl. In other embodiments, the inherent bitter tastant is potassium lactate.

In some embodiments, the method of preparing a edible composition further comprises: (c) adding a bitter tastant. In some embodiments, the bitter tastant is a potassium salt. In some embodiments, the potassium salt is KCl or potassium lactate. In specific embodiments, the potassium salt is KCl. In some embodiments, the bitter tastant is added before the compound of the present invention. In other embodiments, the bitter tastant is added after the compound of the present invention. In some embodiments, the compounds of the present invention are combined with the bitter tastant and then combined with the comestibly acceptable carrier. In other embodiments, the compound of the present invention is combined sequentially with the comestibly acceptable carrier and then the bitter tastant. In yet other embodiments, the compounds of the present invention are combined with a mixture of the bitter tastant and the comestibly acceptable carrier.

In some embodiments, a compound of the invention and the bitter tastant, if present, are mixed with the comestibly acceptable carrier. In other embodiments, the compound and the bitter tastant, if present, are sprayed onto or coat the comestibly acceptable carrier. In some embodiments, the compound of the invention is plated on a carbohydrate or salt, encapsulated on a salt or a carbohydrate (spray dried), or co-crystallized with a potassium salt to create a “topping” salt.

In some embodiments, the bitter tastant is a bitter tasting salt. In some embodiments, the bitter tastant is a potassium salt, a magnesium salt, or a calcium salt. In some embodiments, the bitter tastant is a potassium salt. In some embodiments, the bitter tastant is KCl. In other embodiments, the bitter tastant is potassium lactate.

In some embodiments, the edible composition further comprises a sodium salt. In some embodiments, the edible composition further comprises NaCl. In other embodiments, the edible composition further comprises sodium lactate. In further embodiments, the edible composition further comprises sugar.

In some embodiments, the methods of preparing an edible composition further comprise adding one or more additional components selected from the group consisting of preservatives, nutritives, flavorants or flavor modifiers, which may lack an inherent flavor. In some embodiments, the methods of preparing an edible composition further comprise adding one or more additional flavor modifiers.

In some embodiments, the edible composition is a consumer product.

### Method of Preparing a Food Product

According to another aspect, the invention provides a method of preparing an edible composition, wherein the edible composition is a food product. The method comprises: (a) providing a foodstuff; and (b) adding to the foodstuff of (a) a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof or any one of Compounds 1-58, as described above, or combinations thereof. In some embodiments, the compound of the invention is added in the form of an edible composition comprising the compound of the invention.

In some embodiments, the foodstuff in (a) is inherently bitter. In such embodiments, the food stuff may inherently contain a bitter tastant. In some embodiments, the inherent bitter tastant is a bitter tasting salt. In some embodiments, the inherent bitter tastant is a potassium salt, a magnesium salt, or a calcium salt. In some embodiments, the inherent bitter tastant is a potassium salt. In some embodiments, the inherent bitter tastant is KCl. In other embodiments, the inherent bitter tastant is potassium lactate.

In some embodiments, the method comprises: (a) providing a food product; and (b) adding to the food product of (a) an edible composition comprising compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described above, or combinations thereof. In some embodiments, the compound of the invention is added in the form of an edible composition comprising the compound of the invention.

In some embodiments, the food product in (a) comprises a bitter tastant, in some embodiments, the bitter tastant is a bitter tasting salt. In some embodiments, the bitter tastant is a potassium salt, a magnesium salt, or a calcium salt. In some embodiments, the bitter tastant is a potassium salt. In some embodiments, the bitter tastant is KCl. In other embodiments, the bitter tastant is potassium lactate.

In some embodiments, the method of preparing a food product further comprises: (c) adding a bitter tastant. In some embodiments, the bitter tastant is a potassium salt, such as KCl or potassium lactate. In specific embodiments, the potassium salt is KCl. In some embodiments, the bitter tastant is added before the compound of the present invention. In other embodiments, the bitter tastant is added after the compound of the present invention. In some embodiments, the compound of the invention is added with the bitter tastant. In some embodiments, the compound of the present invention is combined with the bitter tastant and then combined with the foodstuff or food product. In other embodiments, the compound of the present invention is combined sequentially with the foodstuff or food product and then the bitter tastant. In yet other embodiments, the compound of the present invention is combined with a mixture of the bitter tastant and the foodstuff or food product.

In some embodiments, the compound and the bitter tastant, if present, are mixed with the foodstuff. In other embodiments, the compound and the bitter tastant, if present, are sprayed onto or coat the foodstuff. In some embodiments, the compound of the invention is plated on a carbohydrate or salt, encapsulated on a salt or a carbohydrate (spray dried), or co-crystallized with a potassium salt to create a “topping” salt.

In some embodiments, the bitter tastant is a bitter tasting salt. In some embodiments, the bitter tastant is a potassium salt, a magnesium salt, or a calcium salt. In some embodiments, the bitter tastant is a potassium salt. In some embodiments, the bitter tastant is KC. In other embodiments, the bitter tastant is potassium lactate.

In some embodiments, the food product further comprises a sodium salt. In some embodiments, the food product further comprises NaCl. In other embodiments, the food product further comprises sodium lactate. In further embodiments, the food product further comprises sugar.

In some embodiments, the methods of preparing a food product further comprise adding one or more additional components selected from the group consisting of preservatives, nutritives, flavorants or flavor modifiers, which may lack an inherent flavor.

### Method of Preparing a Pharmaceutical Composition

According to another aspect, the invention provides a method of preparing an edible composition, wherein the edible composition is a pharmaceutical composition. The method comprises: (a) providing a pharmaceutically active ingredient; and (b) adding to the pharmaceutically active ingredient of (a) a compound of Formula (I), Formula (IIa), Formula (IIb), Formula (IIIb), Formula (IIIb′), Formula (IIIb″), Formula (IV), Formula (Va), Formula (Vb), Formula (VIa), Formula (Vb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described above, or combinations thereof, with the pharmaceutically active ingredient. In some embodiments, the compound of the invention is added in the form of an edible composition comprising the compound of the invention.

In some embodiments, the pharmaceutically active ingredient in (a) is inherently bitter. In such embodiments, the pharmaceutically active ingredient may inherently contain a bitter tastant. In some embodiments, the inherent bitter tastant is a bitter tasting salt. In some embodiments, the inherent bitter tastant is a potassium salt, a magnesium salt, or a calcium salt. In some embodiments, the inherent bitter tastant is a potassium salt.

In some embodiments, the method of preparing a pharmaceutical composition further comprises: (c) adding a bitter tastant. In some embodiments, the bitter tastant is a potassium salt. In some embodiments, the potassium salt is KCl or potassium lactate. In specific embodiments, the potassium salt is KCl. In some embodiments, the bitter tastant is added before the compound of the present invention. In other embodiments, the bitter tastant is added after the compound of the present invention. In some embodiments, the bitter tastant is added with the compound of the invention. In some embodiments, the compound of the present invention is combined with the bitter tastant and then combined with the pharmaceutically active ingredient. In other embodiments, the compound of the present invention is combined sequentially with the pharmaceutically active ingredient and then the bitter tastant. In yet other embodiments, the compound of the present invention is combined with a mixture of the bitter tastant and the pharmaceutically active ingredient.

In some embodiments, the compound and the bitter tastant, if present, are mixed with the pharmaceutically active ingredient. In other embodiments, the compound and the bitter tastant, if present, are sprayed onto or coat the pharmaceutical composition. In some embodiments, the compound of the invention is encapsulated with the pharmaceutically active ingredient. In some embodiments, the compound of the invention is in a form such that the rate of release is regulated vis a vis the rate of release of the bitter tastant, which in some embodiments is the pharmaceutically active ingredient.

In some embodiments, the bitter tastant is a bitter tasting salt. In some embodiments, the bitter tastant is a potassium salt, a magnesium salt, or a calcium salt. In some embodiments, the bitter tastant is a potassium salt. In some embodiments, the bitter tastant is KCl. In other embodiments, the bitter tastant is potassium lactate.

In some embodiments, the pharmaceutical composition further comprises a sodium salt. In some embodiments, the pharmaceutical composition further comprises NaCl. In other embodiments, the pharmaceutical composition further comprises sodium lactate. In further embodiments, the pharmaceutical composition further comprises sugar.

In some embodiments, the pharmaceutical composition further comprises a pharmaceutically acceptable carrier. Pharmaceutically acceptable carriers that may be used in these compositions include, but are not limited to, ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellose, polyacrylates, waxes, polyethylene-polyoxypropylene-block polymers, polyethylene glycol and wool fat.

In some embodiments, the methods of preparing a pharmaceutical composition further comprise adding one or more additional components selected from the group consisting of preservatives, nutritives, flavorants or flavor modifiers, which may lack an inherent flavor.

### Method of Reducing or Eliminating the Perception of Bitter Taste in a Subject

According to another aspect, the invention provides a method of reducing or eliminating the perception of bitter taste in a subject. The method comprises the use of an edible composition comprising a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described above, or combinations thereof.

The method can be used to reduce or eliminate bitter taste in any edible composition, including a foodstuff, food product, pharmaceutical composition or consumer product. The edible composition may be in any form. In some embodiments, the composition is in the form of, for example, a gum, lozenge, sauce, condiment, meat matrix, meat slurry, paste, suspension, spread, coating, a liquid, a gel, an emulsion, granules, or seasoning.

In some embodiments the edible composition is utilized by, for example, placement in the oral cavity or by ingestion. In some embodiments, the edible composition is placed in the oral cavity or ingested before a bitter food stuff, food product, pharmaceutical composition or consumer product. In some embodiments, the edible composition is placed in the oral cavity or ingested concurrently with a bitter food stuff, food product, pharmaceutical composition or consumer product, either as a separate edible composition or by incorporation in the bitter food stuff, food product, pharmaceutical composition or consumer product. In some embodiments, the edible composition is placed in the oral cavity or ingested after a bitter food stuff, food product, pharmaceutical composition or consumer product. For example, a compound of the invention can be combined with foodstuffs or food products to reduce the bitter taste of a food product. Alternatively, a compound of the invention can be used, for example, in a lozenge or gum for use after exposure to a bitter food stuff, food product, pharmaceutical composition or consumer product (e.g., to reduce or eliminate a bitter aftertaste).

### Method of Reducing the Amount of Sodium in an Edible Composition

According to another embodiment, the invention provides a method of reducing the amount of sodium in an edible composition, such as a food product, a pharmaceutical composition or a consumer product. In some embodiments, the invention provides a method of reducing the amount of a sodium containing compound in an edible composition, such as a food product, a pharmaceutical composition or a consumer product. In another embodiment, the invention provides a method of reducing the amount of NaCl in an edible composition, such as a food product, a pharmaceutical composition or a consumer product. In another embodiment, the invention provides a method of reducing the amount of sodium lactate in an edible composition, such as a food product, a pharmaceutical composition or a consumer product. In some embodiments, the sodium salt is replaced with a non-sodium salt. In some embodiments, the non-sodium salt is a calcium salt, a magnesium salt, or a potassium salt. In some embodiments, the non-sodium salt is a potassium salt.

In some embodiments, the method comprises: (a) replacing an amount of a sodium salt present in an edible composition with an amount of a potassium salt; and (b) incorporating into the edible composition an effective amount of a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb), Formula (XVc), as described herein, or combinations thereof, or any one of Compounds 1-58, as described above, or combinations thereof. In some embodiments, the compound of the invention is added in the form of an edible composition comprising the compound of the invention.

In some embodiments, the method of reducing the amount of sodium in an edible composition comprises the steps of: (a) ingesting a first edible composition, in which an amount of a sodium salt has been replaced with an amount of a potassium salt; and (b) ingesting a second edible compound, which comprises a compound of the invention. In some embodiments, the first edible composition is ingested before the second edible composition. In some embodiments, the first edible composition is ingested after the second edible composition. In some embodiments, the first edible composition is ingested concurrently with the second edible composition.

In some embodiments, the edible composition is a food product. In some embodiments, the edible composition is a pharmaceutical composition. In some embodiments, the edible composition is a consumer product.

In some embodiments, the potassium salt is added to the edible composition prior to addition of an effective amount of a compound of the invention. In some embodiments, the potassium salt is added to the edible composition subsequent to addition of an effective amount of a compound of the invention. In some embodiments, the potassium sail is added to the edible composition concurrent with addition of an effective amount of a compound of the invention.

In some embodiments, the amount of sodium replaced in the edible composition in step (a) is an amount sufficient to maintain or restore the health of a subject. In some embodiments, the amount of sodium replaced in the edible composition is an amount sufficient to decrease hypertension in a subject. In some embodiments, the amount of sodium replaced by potassium in the edible composition is an amount to sufficient to change the texture or freezing point of the edible composition. In some embodiments, the amount of sodium replaced is up to 1%, 2,%, 3%, 4%, 5%, 6%, 7%, 8%, 9%, 10%, 15%, 20%, 25%, 30%, 35%, 40%, 45%, 50%, 55%, 60%, 65%, 70% 75%, 80%, 85%, 90%, 95% or 100%. These amounts are not meant to be limiting, and increments between the recited percentages are specifically envisioned as part of the invention.

In some embodiments, the amount of compound added in step (b) reduces the perception of bitter taste in the subject. The bitter taste is completely reduced or partially reduced. In some embodiments, the perception of salty taste is maintained.

In some embodiments, the amount of compound added in step (b) is sufficient to permit replacement of up to 1%, 2%, 3%, 4%, 5%, 6%, 7%, 8%, 9%, 10%, 15%, 20%, 25%, 30%, 35%, 40%, 45%, 50%, 55%, 60%, 65%, 70% 75%, 80%, 85%, 90%, 95% or 100% of the amount of sodium present in the edible composition with potassium. These amounts are not meant to be limiting, and increments between the recited percentages are specifically envisioned as part of the invention. In some embodiments, the amount of compound added in step (b) is sufficient to permit replacement of up to 25% of the amount of sodium present in the edible composition with potassium. In other embodiments, the amount of compound added in step (b) is sufficient to permit replacement of up to 50% of the amount of sodium present in the edible composition with potassium. In other embodiments, the amount of compound added in step (b) is sufficient to permit replacement of up to 75% of the amount of sodium present in the edible composition with potassium. In yet other embodiments, the amount of compound added in step (b) is sufficient to permit replacement of up to 100% of the amount of sodium present in the edible composition with potassium.

In some embodiments, the method of reducing the amount of sodium in an edible composition further comprises adding one or more additional components selected from the group consisting of preservatives, nutritives, flavorants or flavor modifiers, which may lack an inherent flavor.

In some embodiments, the method comprises: (a) replacing an amount of NaCl present in an edible composition with an amount of KCl; and (b) incorporating into the edible composition an effective amount of a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb). Formula (XIV), Formula (XVa), Formula (XVb) or Formula (XVc), as described herein, or combinations thereof, or any one of Compounds 1-58, as described above, or combinations thereof.

In some embodiments, the method of reducing the amount of sodium in an edible composition comprises the steps of: (a) ingesting a first edible composition, in which an amount of NaCl has been replaced with an amount of KCl; and (b) ingesting a second edible compound, which comprises a compound of the invention. In some embodiments, the first edible composition is ingested before the second edible composition. In some embodiments, the first edible composition is ingested after the second edible composition. In some embodiments, the first edible composition is ingested concurrently with the second edible composition.

In some embodiments, the edible composition is a food product. In some embodiments, the edible composition is a pharmaceutical composition. In some embodiments, the edible composition is a consumer product.

In some embodiments, the KCl is added to the edible composition prior to addition of an effective amount of a compound of the invention. In some embodiments, the KCl is added to the edible composition subsequent to addition of an effective amount of a compound of the invention. In some embodiments, the KCl is added to the edible composition concurrent with addition of an effective amount of a compound of the invention.

In some embodiments, the amount of NaCl replaced by KCl in the edible composition in step (a) is an amount sufficient to maintain or restore the health of a subject. In some embodiments, the amount of NaCl replaced by KCl in the edible composition is an amount sufficient to decrease hypertension in a subject. In some embodiments, the amount of NaCl replaced by KCl in the edible composition is an amount to sufficient to change the texture or freezing point of the edible composition. In some embodiments, the amount of NaCl replaced by KCl is up to 1%, 2%, 3%, 4%, 5%, 6%, 7%, 8%, 9%, 10%, 15%, 20%, 25%, 30%, 35%, 40%, 45%, 50%, 55%, 60%, 65%, 70% 75%, 80%, 85%, 90%, 95% or 100%. These amounts are not meant to be limiting, and increments between the recited percentages are specifically envisioned as part of the invention.

In some embodiments, the amount of compound added in step (b) reduces the perception of bitter taste in the subject. The bitter taste is completely reduced or partially reduced. In some embodiments, the perception of sally taste is maintained.

In some embodiments, the amount of compound added in step (b) is sufficient to permit replacement of up to 1%, 2%, 3%, 4%, 5%, 6%, 7%, 8%, 9%, 10%, 15%, 20%, 25%, 30%, 35%, 40%, 45%, 50%, 55%, 60%, 65%, 70% 75%, 80%, 85%, 90%, 95% or 100% of the amount of NaCl present in the edible composition with KCl. These amounts are not meant to be limiting, and increments between the recited percentages are specifically envisioned as part of the invention. In some embodiments, the amount of compound added in step (b) is sufficient to permit replacement of up to 25% of the amount of NaCl present in the edible composition with KCl. In other embodiments, the amount of compound added in step (b) is sufficient to permit replacement of up to 50% of the amount of NaCl present in the edible composition with KCl. In other embodiments, the amount of compound added in step (b) is sufficient to permit replacement of up to 75% of the amount of NaCl present in the edible composition with KCl. In yet other embodiments, the amount of compound added in step (b) is sufficient to permit replacement of up to 100% of the amount of NaCl present in the edible composition with KCl.

In some embodiments, the method of reducing the amount of NaCl in an edible composition or food product comprises maintaining a salty flavor.

In some embodiments, the method of reducing the amount of NaCl in an edible composition further comprises adding one or more additional components selected from the group consisting of preservatives, nutritives, flavorants or flavor modifiers, which may lack an inherent flavor.

In other embodiments, the method of reducing the amount of sodium in an edible composition or food product comprises: (a) replacing an amount of sodium lactate present in a food product with an amount of potassium lactate; and (b) incorporating into the edible composition an effective amount of a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described above, or combinations thereof.

In some embodiments, the method of reducing the amount of sodium in an edible composition comprises the steps of: (a) ingesting a first edible composition, in which an amount of sodium lactate has been replaced with an amount of potassium lactate; and (b) ingesting a second edible compound, which comprises a compound of the invention. In some embodiments, the first edible composition is ingested before the second edible composition. In some embodiments, the first edible composition is ingested after the second edible composition. In some embodiments, the first edible composition is ingested concurrently with the second edible composition.

In some embodiments, the edible composition is a food product. In some embodiments, the edible composition is a pharmaceutical composition. In some embodiments, the edible composition is a consumer product.

In some embodiments, the potassium lactate is added to the edible composition prior to addition of an effective amount of a compound of the invention. In some embodiments, the potassium lactate is added to the edible composition subsequent to addition of an effective amount of a compound of the invention. In some embodiments, the potassium lactate is added to the edible composition concurrent with addition of an effective amount of a compound of the invention.

In some embodiments, the amount of sodium lactate replaced by potassium lactate in the edible composition in step (a) is an amount sufficient to maintain or restore the health of a subject. In some embodiments, the amount of sodium lactate replaced by potassium lactate in the edible composition is an amount sufficient to decrease hypertension in a subject. In some embodiments, the amount of sodium lactate replaced by potassium lactate in the edible composition is an amount to sufficient to change the texture or freezing point of the edible composition. In some embodiments, the amount of sodium lactate replaced by potassium lactate is up to 1%, 2%, 3%, 4%, 5%, 6%, 7%, 8%, 9%, 10%, 15%, 20%, 25%, 30%, 35%, 40%, 45%, 50%, 55%, 60%, 65%, 70% 75%, 80%, 85%, 90%, 95% or 100%. These amounts are not meant to be limiting, and increments between the recited percentages are specifically envisioned as part of the invention.

In some embodiments, the amount of compound added in step (b) reduces the perception of bitter taste in the subject. The bitter taste is completely reduced or partially reduced. In some embodiments, the perception of salty taste is maintained.

In some embodiments, the amount of compound added in step (b) is sufficient to permit replacement of up to 1%, 2%, 3%, 4%, 5%, 6%, 7%, 8%, 9%, 10%, 15%, 20%, 25%, 30%, 35%, 40%, 45%, 50%, 55%, 60%, 65%, 70% 75%, 80%, 85%, 90%, 95% or 100% of the amount of sodium lactate present in the edible composition with potassium lactate. These amounts are not meant to be limiting, and increments between the recited percentages are specifically envisioned as part of the invention. In some embodiments, the amount of compound added in step (b) is sufficient to permit replacement of up to 25% of the amount of sodium lactate present in the edible composition with potassium lactate. In other embodiments, the amount of compound added in step (b) is sufficient to permit replacement of up to 50% of the amount of sodium lactate present in the edible composition with potassium lactate. In other embodiments, the amount of compound added in step (b) is sufficient to permit replacement of up to 75% of the amount of sodium lactate present in the edible composition with potassium lactate. In yet other embodiments, the amount of compound added in step (b) is sufficient to permit replacement of up to 100% of the amount of sodium lactate present in the edible composition with potassium lactate.

In some embodiments, the method of reducing the amount of sodium lactate in an edible composition or food product comprises maintaining the preservation of the food product.

In some embodiments, the method of reducing the amount of sodium lactate in an edible composition further comprises adding one or more additional components selected from the group consisting of preservatives, nutritives, flavorants or flavor modifiers, which may lack an inherent flavor.

### Method of Reducing the Amount of Sugar in an Edible Composition

According to another embodiment, the invention provides a method of reducing the amount of sugar in an edible composition. In some embodiments, the method comprises: (a) replacing an amount of sugar present in an edible composition with an amount of Acesulfame K; and (b) incorporating into the edible composition an effective amount of a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described above, or combinations thereof.

In some embodiments, the edible composition is a food product. In some embodiments, the edible composition is a pharmaceutical composition. In some embodiments, the edible composition is a consumer product.

In some embodiments, the Acesulfame K is added to the edible composition prior to addition of an effective amount of a compound of the invention. In some embodiments, the Acesulfame K is added to the edible composition subsequent to addition of an effective amount of a compound of the invention. In some embodiments, the Acesulfame K is added to the edible composition concurrent with addition of an effective amount of a compound of the invention.

In some embodiments, the amount of sugar replaced in the edible composition in (a) is an amount sufficient to maintain or restore the health of a subject. In some embodiments, the amount of sugar replaced in the edible composition is an amount sufficient to result in weight loss in a subject. In some embodiments, the amount of sugar replaced by Acesulfame K in the edible composition is an amount to sufficient to alleviate the effects of, or treat, a disease associated with sugar consumption or excessive weight of the subject (e.g., diabetes). In some embodiments, the amount of sugar replaced by Acesulfame K is up to 1%, 2%, 3%, 4%, 5%, 6%, 7%, 8%, 9%, 10%, 15%, 20%, 25%, 30%, 35%, 40%, 45%, 50%, 55%, 60%, 65%, 70% 75%, 80%, 85%, 90%, 95% or 100%. These amounts are not meant to be limiting, and increments between the recited percentages are specifically envisioned as part of the invention.

In some embodiments, the amount of compound added in (b) reduces the perception of bitter taste in the subject. The bitter taste is completely reduced or partially reduced. In some embodiments, the perception of sweet taste is maintained.

In some embodiments, the amount of compound added in step (b) is sufficient to permit replacement of up to 1%, 2%, 3%, 4%, 5%, 6%, 7%, 8%, 9%, 10%, 15%, 20%, 25%, 30%, 35%, 40%, 45%, 50%, 55%, 60%, 65%, 70% 75%, 80%, 85%, 90%, 95% or 100% of the amount of sugar present in the edible composition with Acesulfame K. These amounts are not meant to be limiting, and increments between the recited percentages are specifically envisioned as part of the invention. In some embodiments, the amount of compound added in step (b) is sufficient to permit replacement of up to 25% of the amount of sugar present in the edible composition with Acesulfame K. In other embodiments, the amount of compound added in step (b) is sufficient to permit replacement of up to 50% of the amount of sugar present in the edible composition with Acesulfame K. In other embodiments, the amount of compound added in step (b) is sufficient to permit replacement of up to 75% of the amount of sugar present in the edible composition with Acesulfame K. In yet other embodiments, the amount of compound added in step (b) is sufficient to permit replacement of up to 100% of the amount of sugar present in the edible composition with Acesulfame K.

In some embodiments, the method of reducing the amount of sugar in an edible composition comprises maintaining a sweet flavor.

In some embodiments, the method of reducing the amount of sugar in an edible composition or food product further comprises adding one or more additional components selected from the group consisting of preservatives, nutritives, flavorants or flavor modifiers, which may lack an inherent flavor.

### Method of Reducing Sodium Intake of a Subject

According to another embodiment, the invention provides a method of reducing sodium intake of a subject. In some embodiments, the method comprises the step of providing an edible composition of the present invention to the subject, wherein all or a portion of the sodium salts in the edible composition is replaced with one or more non-sodium salts, and wherein the edible composition comprises a compound of the present invention. In some embodiments, the non-sodium salt is a calcium salt, a magnesium salt, or a potassium salt. In some embodiments, the non-sodium salt is a potassium salt. In some embodiments, the edible composition is a food product. In some embodiments, the edible composition is a pharmaceutical composition. In some embodiments, the edible composition is a consumer product. In some embodiments the sodium salt is NaCl and the potassium salt is KCl. In some embodiments, the sodium salt is sodium lactate and the potassium salt is potassium lactate.

In some embodiments, the methods of reducing sodium intake of a subject further comprise the step of identifying a subject in need thereof. The skilled worker would be able to identify a subject in need of reducing sodium intake. Non-limiting examples of such subjects include subjects that suffer from any one or more of the following disorders: hypernatremia, hypertension, cardiovascular disease, edema, seizures due to cerebral edema, dehydration (due to excess sweating, diarrhea, urinary tract disorders or diuretics), diabetes insipidus, Conn's syndrome, and Cushing's syndrome.

In some embodiments, the amount of the sodium salt replaced by a potassium salt in the edible composition is an amount sufficient to maintain or restore the health of a subject. In some embodiments, the amount of the sodium salt replaced by a potassium salt in the edible composition is an amount sufficient to decrease hypertension in a subject. In some embodiments, the amount of the sodium salt replaced by a potassium salt in the edible composition is up to 1%, 2%, 3%, 4%, 5%, 6%, 7%, 8%, 9%, 10%, 15%, 20%, 25%, 30%, 35%, 40%, 45%, 50%, 55%, 60%, 65%, 70% 75%, 80%, 85%, 90%, 95% or 100%. These amounts are not meant to be limiting, and increments between the recited percentages are specifically envisioned as part of the invention. In some embodiments, a subject's daily sodium intake is less than 2500 mg/day, less than 2000 mg/day, less than 1500 mg/day, less than 1000 mg/day, or less than 500 mg/day, where desirable.

In some embodiments, the amount of the compound of the invention added to the edible composition is sufficient to reduce a subject's sodium intake by up to 1%, 2%, 3%, 4%, 5%, 6%, 7%, 8%, 9%, 10%, 15%, 20%, 25%, 30%, 35%, 40%, 45%, 50%, 55%, 60%, 65%, 70% 75%, 80%, 85%, 90%, 95% or 100%. These amounts are not meant to be limiting, and increments between the recited percentages are specifically envisioned as part of the invention. In some embodiments, the amount of compound of the invention added to the edible composition is sufficient to permit reduction of the subject's sodium intake by up to 25%. In other embodiments, the amount of compound of the invention added to the edible composition is sufficient to permit reduction of the subject's sodium intake by up to 50%. In other embodiments, the amount of compound of the invention added to the edible composition is sufficient to permit reduction of the subject's sodium intake by up to 75%. In yet other embodiments, the amount of compound of the invention added to the edible composition is sufficient to permit reduction of the subject's sodium intake by up to 100%.

In some embodiments, the method of reducing sodium intake of a subject further comprises adding one or more additional components selected from the group consisting of preservatives, nutritives, flavorants or flavor modifiers, which may lack an inherent flavor.

### Method of Reducing Sugar Intake of a Subject

According to another embodiment, the invention provides a method of reducing sugar intake of a subject. In some embodiments, the method comprises the step of providing an edible composition of the present invention to the subject, wherein all or a portion of the sugar in the edible composition is replaced with Acesulfame K, and wherein the edible composition comprises a compound of the present invention. In some embodiments, the edible composition is a food product. In some embodiments, the edible composition is a pharmaceutical composition. In some embodiments, the edible composition is a consumer product.

In some embodiments, the methods of reducing sugar intake of a subject further comprise the step of identifying a subject in need thereof. The skilled worker would be able to identify a subject in need of reducing sugar intake. Non-limiting examples of such subjects include subjects that suffer from any one or more of the following disorders: diabetes, pre-diabetes, insulin resistance, obesity, excessive weight, and hyperglycemia.

In some embodiments, the amount of sugar replaced by Acesulfame K in the edible composition is an amount sufficient to maintain or restore the health of a subject. In some embodiments, the amount of sugar replaced by Acesulfame K in the edible composition is an amount sufficient to result in weight loss in a subject. In some embodiments, the amount of sugar replaced by Acesulfame K in the edible composition is an amount to sufficient to alleviate the effects of, or treat, a disease associated with sugar consumption or excessive weight of the subject (e.g., diabetes). In some embodiments, the amount of sugar replaced by Acesulfame K in the edible composition is up to 1%, 2%, 3%, 4%, 5%, 6%, 7%, 8%, 9%, 10%, 15% 20%, 25%, 30%, 35%, 40%, 45%, 50%, 55%, 60%, 65%, 70% 75%, 80%, 85%, 90%, 95% or 100%. These amounts are not meant to be limiting, and increments between the recited percentages are specifically envisioned as part of the invention. In some embodiments, the subject's daily sugar intake is less than 250 g/day, less than 200 g/day, less than 175 g/day, less than 150 g/day, less than 125 g/day, less than 100 g/day, less than 75 g/day, less than 50 g/day or less than 25 g/day.

In some embodiments, the amount of compound of the invention added to the edible composition is sufficient to permit reduction of a subject's sugar intake by up to 1%, 2%, 3%, 4%, 5%, 6%, 7%, 8%, 9%, 10%, 15%, 20%, 25%, 30%, 35%, 40%, 45%, 50%, 55%, 60%, 65% 70% 75%, 80%, 85%, 90%, 95% or 100%. These amounts are not meant to be limiting, and increments between the recited percentages are specifically envisioned as part of the invention. In some embodiments, the amount of compound of the invention added to the edible composition is sufficient to permit reduction of a subject's sugar intake by up to 25%. In other embodiments, the amount of compound of the invention added to the edible composition is sufficient to permit reduction of a subject's sugar intake by up to 50%. In other embodiments, the amount of compound of the invention added to the edible composition is sufficient to permit reduction of a subject's sugar intake by up to 75%. In yet other embodiments, the amount of compound of the invention added to the edible composition is sufficient to permit reduction of a subject's sugar intake by up to 100%.

In some embodiments, the method of method of reducing sugar intake of a subject further comprises adding one or more additional components selected from the group consisting of preservatives, nutritives, flavorants or flavor modifiers, which may lack an inherent flavor.

### Method of Reducing Bitter Taste of an Edible Composition

According to another embodiment, the invention provides methods of reducing the bitter taste in an edible composition. In some embodiments, the edible composition is a food product. In some embodiments, the edible composition is a pharmaceutical composition. In some embodiments, the edible composition is a consumer product.

In one embodiment, the method comprises: (a) adding an effective amount of a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described above, or combinations thereof, to an edible composition such that bitter taste is reduced.

In alternate embodiments, the method comprises: (a) ingesting an effective amount of a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb). Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described above, or combinations thereof, before, along with, or after the edible composition such that bitter taste is reduced.

In some embodiments, the bitter tastant is a bitter tasting salt. In some embodiments, the bitter tastant is a potassium salt, a magnesium salt, or a calcium salt. In some embodiments, the bitter tastant is a potassium salt. In some embodiments, the bitter tastant is KCl. In other embodiments, the bitter tastant is potassium lactate. In some embodiments, the bitter tastant is inherent in the edible composition, such as in an inherently bitter foodstuff.

In some embodiments, the bitter taste is reduced by up to 1%, 2%, 3%, 4%, 5%, 6%, 7%, 8%, 9%, 10%, 15%, 20%, 25%, 30%, 35%, 40%, 45%, 50%, 55%, 60%, 65%, 70% 75%, 80%, 85%, 90%, 95% or 100%. These amounts are not meant to be limiting, and increments between the recited percentages are specifically envisioned as part of the invention. In some embodiments, the bitter taste is reduced by up to 25%. In other embodiments, the bitter taste is reduced by up to 50%. In other embodiments, the bitter taste is reduced by up to 75%. In other embodiments, the bitter taste is reduced by up to 100%.

In some embodiments, the method of reducing the bitter taste attributed to a bitter tastant in an edible composition further comprises adding one or more additional components selected from the group consisting of preservatives, nutritives, flavorants or flavor modifiers, which may lack an inherent flavor.

### Method of Preserving an Edible Composition

According to another embodiment, the invention provides a method of preserving an edible composition or extending the shelf life of an edible composition comprising:
(a) providing an edible composition; and
(b) combining with the edible composition of (a) a preservative and an effective amount of compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof.

In another embodiment, the method of preserving or extending the shelf life of an edible composition comprises:
(a) providing an edible composition; and
(b) combining with the edible composition of (a) a preservative and an effective amount of any one of Compounds 1-58, or combinations thereof.

According to the invention, the preservative can be any bitter-tasting preservative. In some embodiments, the preservative in (a) is a potassium salt. In some embodiments, the preservative in (a) is potassium lactate.

In some embodiments, the edible composition is a food product. In some embodiments, the edible composition is a pharmaceutical composition. In some embodiments, the edible composition is a consumer product.

In some embodiments, the method of preserving an edible composition further comprises adding one or more additional components selected from the group consisting of preservatives, nutritives, flavorants or flavor modifiers, which may lack an inherent flavor.

Method of Reducing the Amount of Sodium in an Edible Composition while Preserving the Edible Composition

According to another embodiment, the invention provides a method of reducing the amount of sodium in an edible composition while preserving the edible composition. In some embodiments, the method comprises replacing an amount of sodium containing preservative present in an edible composition with an amount of potassium containing preservative and adding an effective amount of a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described above, or combinations thereof.

In some embodiments, the method comprises replacing an amount of sodium lactate present in an edible composition with an amount of potassium lactate and adding an effective amount of a compound of Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described above, or combinations thereof.

In some embodiments, the edible composition is a food product. In some embodiments, the edible composition is a pharmaceutical composition. In some embodiments, the edible composition is a consumer product.

In some embodiments, the effective amount of the compound is sufficient to permit reduction of the amount of sodium lactate typically present in an edible composition by up to 1%, 2%, 3%, 4%, 5%, 6%, 7%, 8%, 9%, 10%, 15%, 20%, 25%, 30%, 35%, 40%, 45%, 50%, 55%, 60%, 65%, 70% 75%, 80%, 85%, 90%, 95% or 100%. These amounts are not meant to be limiting, and increments between the recited percentages are specifically envisioned as part of the invention. In some embodiments, the effective amount of the compound is sufficient to permit reduction of the amount of sodium lactate typically present in an edible composition by up to 25%. In other embodiments, the effective amount of the compound is sufficient to permit reduction of the amount of sodium lactate typically present in an edible composition by up to 50%. In other embodiments, the effective amount of the compound is sufficient to permit reduction of the amount of sodium lactate typically present in an edible composition by up to 75%. In yet other embodiments, the effective amount of the compound is sufficient to permit reduction of the amount of sodium lactate typically present in an edible composition by up to 100%.

In some embodiments, the method of reducing the bitter taste attributed to a bitter tastant in an edible composition further comprises adding one or more additional components selected from the group consisting of preservatives, nutritives, flavorants or flavor modifiers, which may lack an inherent flavor. In some embodiments, the method of reducing the amount of sodium lactate in an edible composition while preserving the food product further comprises adding one or more additional flavor modifiers.

### Method of Inhibiting a Bitter Taste Receptor

According to another embodiment, the invention provides a method of inhibiting or reducing activation and/or signaling of a bitter taste receptor. In some embodiments, the method comprises contacting a bitter taste receptor with a compound according to Formula (I), Formula (IIa), Formula (IIb), Formula (IIIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof, or any one of Compounds 1-58, as described above, or combinations thereof.

In some embodiments, the method comprises contacting a bitter taste receptor with an edible composition comprising a compound according to Formula (I), Formula (IIa), Formula (IIb), Formula (IIb), Formula (IIIb′), Formula (IIIb″), Formula (IV), Formula (Va), Formula (Vb), Formula (VIa), Formula (VIb), Formula (VIIa), Formula (VIIb), Formula (VIII), Formula (IX), Formula (X), Formula (XI), Formula (XIIa), Formula (XIIb), Formula (XIIIa), Formula (XIIIb), Formula (XIV), Formula (XVa), Formula (XVb) or Formula (XVc), as described herein, or combinations thereof or any one of Compounds 1-58, as described above, or combinations thereof.

In some embodiments, the edible composition is a food product. In some embodiments, the edible composition is a pharmaceutical composition. In some embodiments, the edible composition is a consumer product.

In some embodiments, the bitter taste receptor is an ex viva receptor present in, for example, an assay. In some embodiments, the bitter taste receptor is an in vitro receptor present in, for example, an assay. In other embodiments, the bitter taste receptor is an in viva receptor present in a subject. In some embodiments, the bitter taste receptor is present in the oral cavity or gastrointestinal tract of a subject. In some embodiments, the bitter receptor is in the oral cavity of a human. In some embodiments, the bitter receptor is in the oral cavity of a non-human animal. In some embodiments, the bitter receptor is in the oral cavity of an animal model.

### Preparation of the Compounds of the Invention

In some embodiments, one or more of the compounds of Formula (I), Formula (IIa), Formula (IIb), Formula (IIIb), Formula (IIIb′), or Formula (IIIb″), as described herein, is commercially available, for example from commercial sources such as ChemBridge Corporation of San Diego, Calif., USA; Sigma-Aldrich® of St., Louis, Mo., USA; TCI America, Portland, Oreg., USA; and Acros Organics, Geel, Belgium; among others.

In other embodiments, one or more of the compounds of Formula (I), Formula (IIa), Formula (IIb), Formula (IIIb), Formula (IIIb′), or Formula (IIIb″) is prepared from commercially available reagents by routine methods in synthetic organic chemistry.

In one embodiment, one or more compounds of Formula (I) or Formula (IIa), wherein X is O, is prepared by nucleophilic displacement of leaving group LG of A2 with the phenoxide anion of A1, generated under basic conditions, to give ether product P1 (Scheme I):

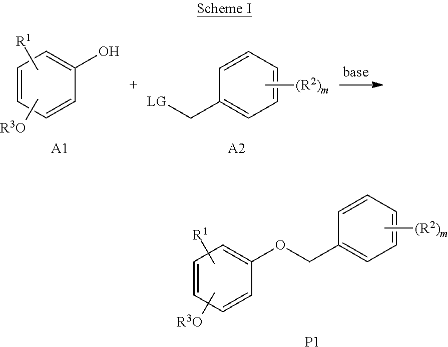

Suitable leaving groups include those recognized in the art, such as halide (e.g., chloro, bromo, iodo), triflate, mesylate, tosylate, and the like. Suitable bases include those recognized in the art for such reactions, and include but are not limited to alkaline and alkaline earth metal hydroxides (such as NaOH, LiOH, etc.), carbonates (such as Na₂CO₃, K₂CO₃, CaCO₃, etc.), and bicarbonates (such as NaHCO₃, KHCO₃, etc.). Other suitable bases include amine bases, such as ammonia, ammonium hydroxide, triethylamine, pyridine, piperidine, pyrrolidine, 2,6-lutidine, 1,8-diazabicycloundec-7-ene (DBU), 4-(dimethylamino)-pyridine, etc.

In another embodiment, one or more compounds of Formula (I) or Formula (IIa), wherein X is NR^{a} and R^{a} is absent, is prepared by imine formation between phenylamine A3 and aldehyde A4, under conditions known in the art to give product P2, for example, conditions employing dehydrating agents, such as molecular sieves (Scheme II):

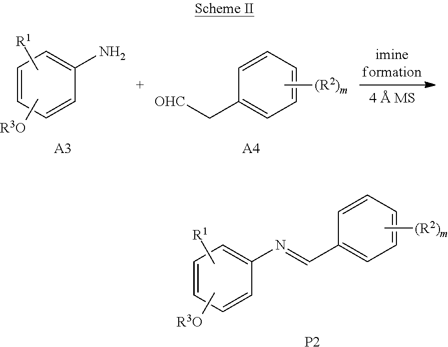

In certain embodiments, one or more compounds of Formula (I) or Formula (IIb) is prepared by reduction of imine P2 to generate amine P3 (Scheme III):

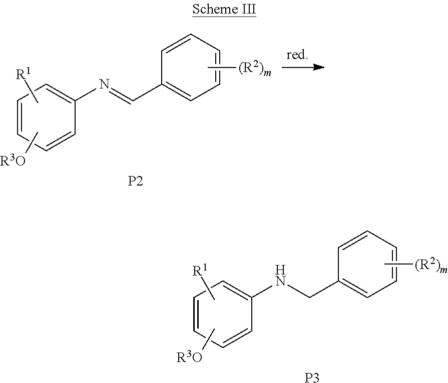

Suitable reducing conditions include those known in the art for reducing imines and iminimum ions, such as hydrogenolysis with hydrogen and palladium, such as palladium on carbon. Another suitable source of hydrogen includes formic acid.

In further embodiments, one or more compounds of Formula (I), wherein X is NR^{a} and R^{a} is not absent, is prepared by reductive alkylation of amine P3 in the presence of the corresponding aldehyde RCHO to form product P4, wherein R^{a} is —CH₂R (Scheme IV):

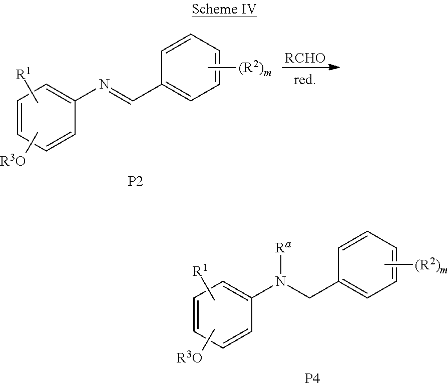

Suitable reductive alkylation conditions include those known in the art for reducing imines and iminimum ions, such as hydrogenolysis with hydrogen and palladium, such as palladium on carbon. Another suitable source of hydrogen includes formic acid.

In some embodiments, one or more of the compounds of Formula (IV), Formula (Va), Formula (VIa), Formula (VIIa), Formula (Vb), Formula (VIb), or Formula (VIIb), as described herein, is commercially available, for example from commercial sources such as ChemBridge Corporation of San Diego, Calif., USA; Sigma-Aldrich® of St. Louis, Mo., USA; TCI America, Portland, Oreg., USA; and Acros Organics, Geel, Belgium; among others.

In other embodiments, one or more of the compounds of Formula (IV) is prepared from commercially available reagents by routine methods in synthetic organic chemistry.

In one embodiment, one or more compounds of Formula (IV), is prepared by acylation of amine A12 with acyl compound A11 bearing leaving group LG to afford amide P11 (Scheme V):

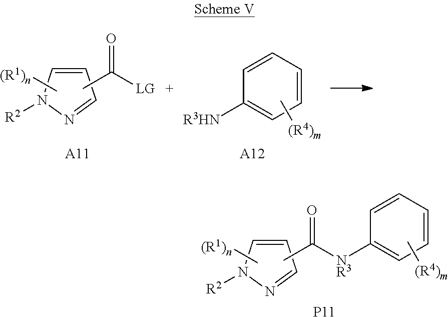

Suitable leaving groups include those recognized in the art for acylation reactions, such as halide (e.g., chloro, bromo, iodo), alkoxy, aryloxy, activated leaving groups, and the like. In some embodiments, acylation conditions also employ an inorganic or organic base. Suitable bases include those recognized in the art for such reactions, and include but are not limited to alkaline and alkaline earth metal carbonates (such as Na₂CO₃, K₂CO₃, CaCO₃, etc.) and bicarbonates (such as NaHCO₃, KHCO₃, etc.). Other suitable bases include aprotic amine bases, such as triethylamine, pyridine, 2,6-lutidine, 1,8-diazabicyclounudec-7-ene (DBU), 4-(dimethylamino)-pyridine, etc.

In one particular embodiment, compound A11 is an acid halide, such as an acid chloride or bromide, and the acylation reaction proceeds in the presence of an aprotic amine base, such as triethylamine, pyridine, 2,6-lutidine, 1,8-diazabicycloundec-7-ene (DBU), 4-(dimethylamino)-pyridine.

Compound A11 can be prepared from the corresponding carboxylic acid using routine methods known in the art.

In some embodiments, one or more of the compounds of Formula (VIII), Formula (IX), or Formula (X), as described herein, is commercially available, for example from commercial sources such as ChemBridge Corporation of San Diego, Calif., USA; Sigma-Aldrich® of St, Louis, Mo., USA; TCI America, Portland, Oreg., USA; and Acros Organics, Geel, Belgium; among others.

In other embodiments, one or more of the compounds of Formula (VIII), Formula (IX), or Formula (X) is prepared from commercially available reagents by routine methods in synthetic organic chemistry.

In one embodiment, one or more compounds of Formula (VIII), Formula (IX), or Formula (X) is prepared by a multi-step sequence beginning with condensing amine A21 and aryl or heteroaryl aldehyde A22 to afford imine (when R^{a} is H in A21) or iminium ion (when R^{a} is not H in A21) P21, which then undergoes [4+2]cycloaddition with cyclic alkene A23 followed by rearomatization to afford fused tricyclic system P22 (Scheme VI):

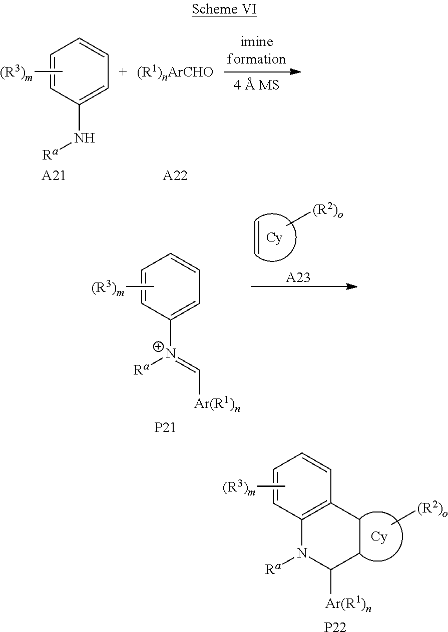

Suitable conditions for imine or iminium ion formation may employ dehydrating agents, such as molecular sieves.

Suitable cycloaddition conditions may include heating, for example, up to at least about 50, 75, 100, 120, 150° C. or greater. In some embodiments, cyclyoaddition conditions include the use of Lewis acids, for example boron compounds (e.g., Bu₂BOTf or BF₃.Et₂O), titanium compounds (e.g., TiCl₄ or titanium alkoxides), aluminum compounds (e.g., AlCl₃ or aluminum alkoxides), silicon compounds (e.g., trialkylsilyl triflates, such as TMS-OTf, trialkylsilyl halides, etc.), and the like, particularly if ring Cy includes an electron withdrawing group (e.g., esters, ketones, aldehydes, cyano, nitro, etc.) in conjugation with the olefin of Cy.

In certain instances, rearomatization is assisted by the use of a base. Suitable bases include those recognized in the art for such reactions, and include but are not limited to alkaline and alkaline earth metal hydroxides (such as NaOH, LiOH, etc.), carbonates (such as Na₂CO₃, K₂CO₃, CaCO₃, etc.), and bicarbonates (such as NaHCO₃, KHCO₃, etc.). Other suitable bases include amine bases, such as ammonia, ammonium hydroxide, triethylamine, pyridine, piperidine, pyrrolidine, 2,6-lutidine, 1,8-diazabicycloundec-7-ene (DBU), 4-(dimethylamino)-pyridine, etc.

In one particular embodiment, one or more compounds of Formula (VIII), Formula (IX), or Formula (X) is prepared first by formation of imine or iminium ion P21, as noted above, followed by [4+2]cycloaddition with cyclopentadiene A24 and then rearomatization, as noted above, to afford fused tricyclic system P23 (Scheme VII):

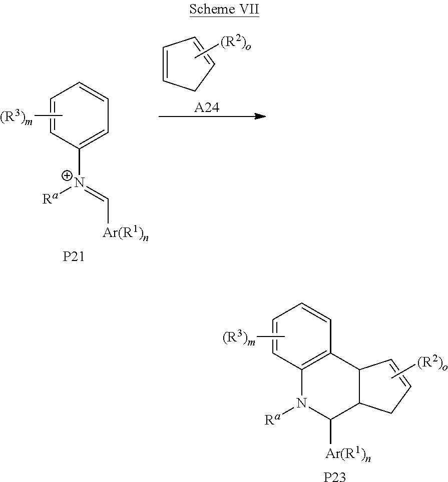

In some embodiments, one or more of the compounds of Formula (XI), Formula (XIIa), Formula (XIIb), Formula (XIIIa), or Formula (XIIb), as described herein, is commercially available, for example from commercial sources such as ChemBridge Corporation of San Diego, Calif., USA; Sigma-Aldrich® of St. Louis, Mo., USA; TCI America, Portland, Oreg., USA; and Acros Organics, Geel, Belgium; among others.

In other embodiments, one or more of the compounds of Formula (XI), Formula (XIIa), Formula (XIIb), Formula (XIIIa), or Formula (XIIIb) is prepared from commercially available reagents by routine methods in synthetic organic chemistry.

In one embodiment, one or more compounds of Formula (XI), Formula (XIIa), or Formula (XIIIa) is prepared by displacement of the leaving group LG of arylamine A31 with a nucleophilic group of hetererocyclic compound A32 to afford product P31 (Scheme VIII):

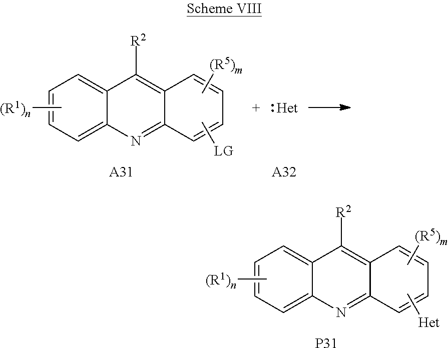

Suitable leaving groups include those recognized in the art for such displacement reactions, such as halide (e.g., fluoro, chloro, bromo, iodo), alkoxy, aryloxy, triflate, mesylate, tosylate, and the like. In some embodiments, the displacement conditions also employ an inorganic or organic base. Suitable bases include those recognized in the art for such reactions, and include but are not limited to alkaline and alkaline earth metal hydroxides (such as NaOH, LiOH, etc.), carbonates (such as Na₂CO₃, K₂CO₃, CaCO₃, etc.), and bicarbonates (such as NaHCO₃, KHCO₃, etc.). Other suitable bases include amine bases, such as ammonia, ammonium hydroxide, triethylamine, pyridine, piperidine, pyrrolidine, 2,6-lutidine, 1,8-diazabicycloundec-7-ene (DBU), 4-(dimethylamino)-pyridine, etc. In some instances, suitable bases include strong bases such as alkoxides (such as sodium or potassium tert-butoxide), lithium diisopropyl amide (LDA), lithium bis(trimethylsilyl)amide (LiIHMDS), sodium bis(trimethylsilyl)amide (NaHMDS), and the like.

In one particular embodiment, one or more compounds of Formula (XI), Formula (XIIa), or Formula (XIIIa) is prepared by displacement of the leaving group LG of arylamine A33 with an amine of hetererocyclic compound A34 under strongly basic conditions to afford product P32 (Scheme IX):

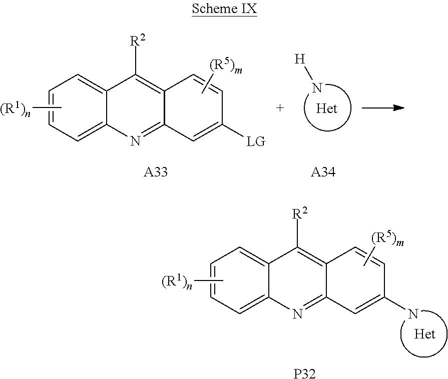

Suitable leaving groups include those recognized in the art for such displacement reactions, such as halide (e.g., fluoro, chloro, bromo, iodo), alkoxy, aryloxy, triflate, mesylate, tosylate, and the like. In some embodiments, suitable basic conditions employ an inorganic or organic base. Suitable strong bases include alkoxides (such as sodium or potassium tert-butoxide), lithium diisopropyl amide (LDA), lithium bis(trimethylsilyl)amide (LiHMDS), sodium bis(trimethylsilyl)amide (NaHMDS), and the like.

In another embodiment, one or more compounds of Formula (XI), Formula (XIIb), or Formula (XIIIb) is prepared by displacement of the leaving group LG of arylamine A35 with a nucleophilic group of hetererocyclic compound A32 to afford product P33 (Scheme X):

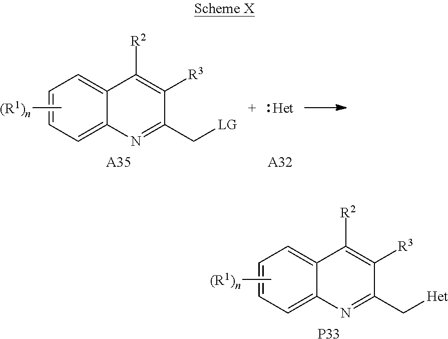

Suitable leaving groups include those recognized in the art for such displacement reactions, such as halide (e.g., chloro, bromo, iodo), alkoxy, aryloxy, triflate, mesylate, tosylate, and the like. In some embodiments, the displacement conditions also employ an inorganic or organic base. Suitable bases include those recognized in the art for such reactions, and include but are not limited to alkaline and alkaline earth metal hydroxides (such as NaOH, LiOH, etc.), carbonates (such as Na₂CO₃, K₂CO₃, CaCO₃, etc.), and bicarbonates (such as NaHCO₃, KHCO₃, etc.). Other suitable bases include amine bases, such as ammonia, ammonium hydroxide, triethylamine, pyridine, piperidine, pyrrolidine, 2,6-lutidine, 1,8-diazabicycloundec-7-ene (DBU), 4-(dimethylamino)-pyridine, etc. In some instances, suitable bases include strong bases such as alkoxides (such as sodium or potassium tert-butoxide), lithium diisopropyl amide (LDA), lithium bis(trimethylsilyl)amide (LiHMDS), sodium bis(trimethylsilyl)amide (NaHMDS), and the like.

In a particular embodiment, one or more compounds of Formula (XI), Formula (XIIb), or Formula (XIIIb) is prepared by displacement of the leaving group LG of arylamine A35 with an amine of hetererocyclic compound A36 under basic conditions to afford product P34 (Scheme XI):

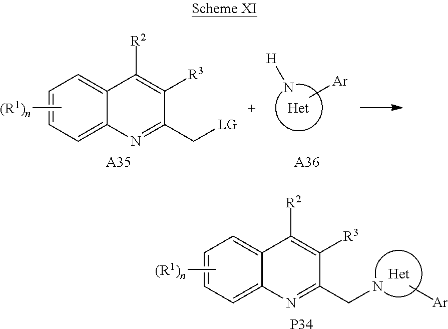

Suitable leaving groups include those recognized in the art for such displacement reactions, such as halide (e.g., chloro, bromo, iodo), alkoxy, aryloxy, triflate, mesylate, tosylate, and the like. In some embodiments, suitable basic conditions employ an inorganic or organic base. Suitable bases include those recognized in the art for such reactions, and include but are not limited to alkaline and alkaline earth metal hydroxides (such as NaOH, LiOH, etc.), carbonates (such as Na₂CO₃, K₂CO₃, CaCO₃, etc.), and bicarbonates (such as NaHCO₃, KHCO₃, etc.). Other suitable bases include amine bases, such as ammonia, ammonium hydroxide, triethylamine, pyridine, piperidine, pyrrolidine, 2,6-lutidine, 1,8-diazabicycloundec-7-ene (DBU), 4-(dimethylamino)-pyridine, etc. In some instances, suitable bases include strong bases such as alkoxides (such as sodium or potassium tert-butoxide), lithium diisopropyl amide (LDA), lithium bis(trimethylsilyl)amide (LiHMDS), sodium bis(trimethylsilyl)amide (NaHMDS), and the like.

In some embodiments, one or more compounds of Formula (XI), Formula (XIIa), Formula (XIIb), Formula (XIIIa), Formula (XIIIb), or even compounds A31, A33, or A35 is prepared by a transition metal-catalyzed coupling reaction of compound A37, bearing leaving group LG, with metallo- or boro-compound A38, wherein M is a metal or boron group, under the appropriate coupling conditions known in the art, to afford coupling product P35 (Scheme XII):

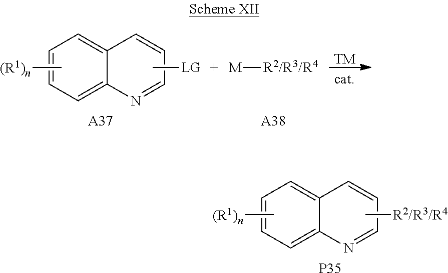

Alternatively, one or more compounds of Formula (XI), Formula (XIIa), Formula (XIIb), Formula (XIIIa), Formula (XIIIb), or even compounds A31, A33, or A35 is prepared by a transition metal-catalyzed coupling reaction of metallo- or boro-compound A39, wherein M is a metal or boron group, with compound A310, bearing leaving group LG, under the appropriate coupling conditions known in the art, to afford coupling product P35 (Scheme XIII):

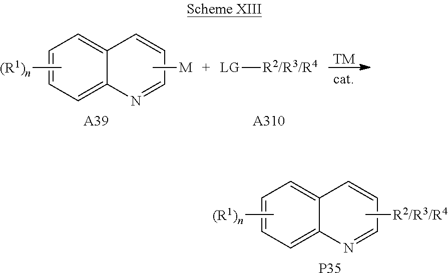

Suitable transition metal catalysts include those derived from palladium, such as Pd(PPh₃)₄, or other noble transition metals. Suitable leaving groups include those recognized in the art, such as halides (e.g., chloro, bromo, iodo), triflates, mesylates, tosylates, and the like. Suitable metal groups include tin, zinc, magnesium, copper, or other metals known to undergo transmetallation with palladium or other noble transition metals.

In certain embodiments, substituted quinoline compounds, such as A35, A37, A39, P33, P34, and P35 maybe prepared by methods known in the art, for example, such as those described in U.S. Pat. No. 6,297,258, which is incorporated by reference herein.

In some embodiments, one or more of the compounds of Formula (XIV), Formula (XVa), Formula (XVb), or Formula (XVc), as described herein, is commercially available, for example from commercial sources such as ChemBridge Corporation of San Diego, Calif., USA; Sigma-Aldrich® of St. Louis, Mo., USA; TCI America, Portland, Oreg., USA; and Acros Organics, Geel, Belgium; among others.

In other embodiments, one or more of the compounds of Formula (XIV), Formula (XVa), Formula (XVb), or Formula (XVc) is prepared from commercially available reagents by routine methods in synthetic organic chemistry.

In one embodiment, one or more compounds of Formula (XIV) is prepared by acylation of amine A41 with acyl compound A42 bearing leaving group LG to afford product P41 (Scheme I):

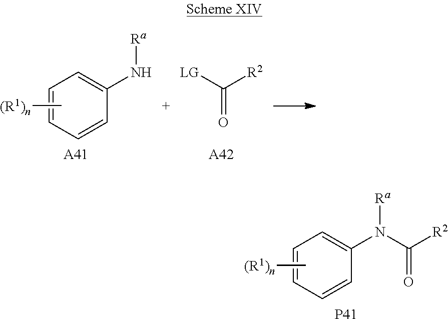

Suitable leaving groups include those recognized in the art for acylation reactions, such as halide (e.g., chloro, bromo, iodo), alkoxy, aryloxy, leaving groups associated with activated esters (e.g., N-succinamide), and the like. In certain embodiments, acyl compound A42 is an acid anhydride; that is LG is —OC(O)R². In some embodiments, acylation conditions also employ an inorganic or organic base. Suitable bases include those recognized in the art for such reactions, and include but are not limited to alkaline and alkaline earth metal hydroxides (such as NaOH, LiOH, etc.), carbonates (such as Na₂CO₃, K₂CO₃, CaCO₃, etc.), and bicarbonates (such as NaHCO₃, KHCO₃, etc.). Other suitable bases include amine bases, such as ammonia, ammonium hydroxide, triethylamine, pyridine, piperidine, pyrrolidine, 2,6-lutidine, 1,8-diazabicycloundec-7-ene (DBU), 4-(dimethylamino)-pyridine, etc.

In one particular embodiment, compound A42 is an acid halide, such as an acid chloride or bromide, and the acylation reaction proceeds in the presence of an amine base, such as triethylamine, pyridine, piperidine, pyrrolidine, 2,6-lutidine, 1,8-diazabicycloundec-7-ene (DBU), 4-(dimethylamino)-pyridine, etc.

In another embodiment, compound A42 is an activated ester and acylation proceeds under mild conditions that do not result in the generation of strong acids.

Compound A42 can be prepared from the corresponding carboxylic acid using routine methods known in the art.

In certain embodiments, compound A41, wherein R^{a} is —CH₂R, is prepared in a two-step sequence, first by imine formation between amine A43 and aldehyde A44 to give imine P42, second by reduction of imine P42 to give A41 (Scheme XV):

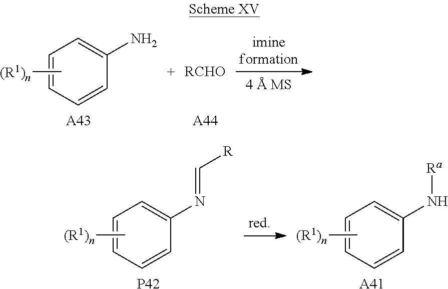

Suitable conditions for imine formation may employ dehydrating agents, such as molecular sieves. Suitable reducing conditions include those known in the art for reducing imines and iminimum ions, such as hydrogenolysis with hydrogen and palladium, such as palladium on carbon. Another suitable source of hydrogen includes formic acid.

The skilled artisan will appreciate that aryl and/or heteroaryl, alkenyl, alkynyl, aralkyl, heteroaralkyl, allyl, and propargyl moieties herein may be readily coupled directly using Stille, Suzuki, Heck, Negishi, Sonongashira, Kumada, Glaser, or other related reactions, such as palladium-mediated cross-coupling reactions. Aryl and/or heteroaryl moieties herein may also be readily coupled through a heteroatom, e.g., using reactions such as the Ullmann reaction, any of various palladium-mediated reactions developed by S. Buchwald and others, by nucleophilic aromatic substitution, or other such reactions. Similarly, amines, alcohols, thiols, and other such heteroatom-bearing compounds herein may be coupled to aryl and/or heteroaryl moieties using palladium-mediated reactions developed by S. Buchwald and others, nucleophilic aromatic substitution, etc. Aryl and/or heteroaryl moieties linked by substituted or unsubstituted hydrocarbon chains herein may also be prepared by Stille, Suzuki, Heck, Friedel-Crafts, and other reactions as will be apparent to those of skill in the art.

It will be understood that the various substituents on the compounds in the above syntheses can be protected from the reaction conditions as necessary using the proper protecting groups, such as those disclosed in Greene, T. W.; Wuts, P. G. M. Greene's Protective Groups in Organic Synthesis, 4th ed.; Wiley-Interscience: New York, 2006.

### EXAMPLES

In order that this invention be more fully understood, the following examples are set forth. These examples are for the purpose of illustration only and are not to be construed as limiting the scope of the invention in any way.

The test compounds used in the following examples were obtained from commercial vendors for synthetic and natural compounds, including VitasM, ChemDiv, ChemBridge, Chromadex, Sigma Aldrich, Penta, Spectrum Chemical, Vigon, and Indofine.

The taste test panelists used in the following examples were screened based upon and selected for their ability to perceive the bitter taste associated with potassium chloride. Only panelists capable of perceiving bitter taste participated in the following taste tests.

Due to the complex nature of taste perception in subjects and the inherently subjective nature of the following experiments, individual taste test trials may yield different results for a given compound. The data presented in the following Examples is illustrative of the taste testing results observed. It is noted that the data presented in the Figures represents a subset of the data presented in the Examples below.

The taste testing experiments below were conducted with panels of varying size (i.e., panels comprising varying numbers of panelists).

### Example 1

### Generation of KCl Test Solutions

Edible KCl solution compositions (“KCl test solutions”) were prepared by first dissolving varying amounts of the test compounds in an amount of ethanol or water (depending on the solubility of the compound) to create a 5 mg/mL stock compound solution. An amount of this stock compound solution is then added to an aqueous KCl solution. Enough EtOH is then added to the resulting stock compound/KCl solution so that the final KCl test solution contains 1% EtOH. KCl solution standards were similarly prepared by dissolving various amounts of KCl in water and ethanol without adding any test compound. NaCl solution standards were similarly prepared by dissolving various amounts of NaCl in water and ethanol without adding any test compound (NaCl solution standards did not contain any KCl).

TABLE 1
KCl Taste Test Solutions

| | | | | Conc. Where |
|---|---|---|---|---|
| | | | Conc. Where | Decrease in |
| | | Conc. of | Decrease in | Bitter Taste |
| Com- | Conc. | Compound | Bitter Taste | Discerned and |
| pound | Of | Tested | Was Discerned | p ≦ 0.1 |
| No. | KCl | (ppm) | (ppm) | (ppm) |
| 1 | 4.85 g/L | 1; 10 | 1; 10 | 1; 10 |
| 2 | 4.85 g/L | 1; 10 | 1; 10 | 1 |
| 3 | 4.85 g/L | 2 | 2 | 2 |
| 4 | 4.85 g/L | 1; 10 | 1; 10 | 10 |
| 5 | 4.85 g/L | 1; 10 | 10 | — |
| 6 | 4.85 g/L | 1; 10 | 1 | — |
| 7 | 4.85 g/L | 1; 10 | 1 | 1 |
| 8 | 4.85 g/L | 1; 10 | 1 | — |
| 9 | 4.85 g/L | 1; 10 | 1; 10 | — |
| 10 | 4.85 g/L | 1; 10 | 1; 10 | 1 |
| 11 | 4.85 g/L | 1; 10 | — | — |
| 12 | 4.85 g/L | 1; 10 | 1; 10 | 10 |
| 13 | 4.85 g/L | 1; 10 | — | — |
| 14 | 4.85 g/L | 1; 10 | 1; 10 | 1 |
| 15 | 4.85 g/L | 1; 10 | 1; 10 | 10 |
| 16 | 4.85 g/L | 1; 10 | 1; 10 | — |
| 17 | 4.85 g/L | 1; 10 | 1 | — |
| 18 | 4.85 g/L | 1; 10 | 1; 10 | — |
| 19 | 4.85 g/L | 1; 10 | 1 | 1 |
| 20 | 4.85 g/L | 1; 10 | 1; 10 | 1 |
| 21 | 4.85 g/L | 1; 10 | — | — |
| 22 | 4.85 g/L | 1; 10 | 1; 10 | 1 |
| 23 | 4.85 g/L | 40 | 40 | 40 |
| 24 | 4.85 g/L | 1; 10; 30 | 1; 10 | — |
| 25 | 4.85 g/L | 1; 10; 30 | 1; 10; 30 | 1; 10 |
| 26 | 4.85 g/L | 1; 10; 30 | 1; 30 | — |
| 27 | 4.85 g/L | 1; 10; 30 | 1; 10; 30 | — |
| 28 | 4.85 g/L | 1; 10 | 1; 10 | 1; 10 |
| 29 | 4.85 g/L | 1; 10 | 1; 10 | 10 |
| 30 | 4.85 g/L | 1; 10 | 1; 10 | — |
| 31 | 4.85 g/L | 1; 10 | 1; 10 | — |
| 32 | 4.85 g/L | 1; 10 | 1; 10 | — |
| 33 | 4.85 g/L | 1; 10 | 1 | — |
| 34 | 4.85 g/L | 1; 10 | 1; 10 | — |
| 35 | 4.85 g/L | 1; 10 | 1 | 1 |
| 36 | 4.85 g/L | 1; 10 | 1; 10 | 10 |
| 37 | 4.85 g/L | 1 | 1 | 1 |
| 38 | 4.85 g/L | 1 | 1 | 1 |
| 39 | 4.85 g/L | 1; 10; 30 | 1; 10; 30 | 1; 10; 30 |
| 40 | 4.85 g/L | 1; 10; 30 | 1; 10; 30 | 10; 30 |
| 41 | 4.85 g/L | 1; 10 | 1; 10 | — |
| 42 | 4.85 g/L | 1; 10 | 1; 10 | — |
| 43 | 4.85 g/L | 1; 10 | 1 | — |
| 44 | 4.85 g/L | 10 | 10 | 10 |
| 45 | 4.85 g/L | 0.1; 1; 10 | 0.1; 1 | 0.1 |
| 46 | 4.85 g/L | 1; 10; 30 | 1; 10; 30 | 1 |
| 47 | 4.85 g/L | 1; 10 | 1; 10 | 1; 10 |
| 48 | 4.85 g/L | 1; 10 | 1; 10 | 1 |
| 49 | 4.85 g/L | 1; 10; 30 | 30 | 30 |
| 50 | 4.85 g/L | 1; 10; 30 | 1 | — |
| 51 | 4.85 g/L | 1; 10; 15; 30 | 15 | 15 |
| 52 | 4.85 g/L | 1; 10; 30 | 10; 30 | 10 |
| 53 | 4.85 g/L | 25 | 25 | 25 |
| 54 | 4.85 g/L | 1; 10; 30 | 1; 30 | — |
| 55 | 4.85 g/L | 1; 10; 30 | 1; 10; 30 | 10; 30 |
| 56 | 4.85 g/L | 1; 10; 30 | 1; 10; 30 | 1 |
| Stan- | 4.85 g/L | — | | |
| dard | | | | |

### Example 2

### Effect of Test Compounds on the Perception of Bitter Taste of Aqueous KCl Solutions in Humans

The effect of the test compounds on the perception of the bitter taste of an aqueous solution of KCl in humans was evaluated using a “sip and Spit” test as follows.

A set of KCl solutions standards was developed and each standard solution was assigned a bitterness taste score of 0-15 (corresponding to aqueous KCl concentrations of 0 mM-120 mM). Panelists were trained to recognize these standards. In addition, before each day of testing, panelists were tested to see if they could determine differences in taste between the standard solutions, if a panelist was unable to recognize a change in KCl concentration, they were excluded from the panel for that day.

In a blind taste test, panelists were asked to compare the bitter taste of a small quantity (e.g. 8 ml) of each of the KCl Test Solutions to the taste of a KCl solution standard, without swallowing (see, e.g., Table 1). Specifically, panelists were asked to rate the bitterness of each KCl Test Solution on a scale of 0-15 using the same scale developed for the KCl solution standards. Each sample was tested in 2-4 discrete taste test experiments. Panelists were asked to rinse with water, eat a cracker, and wait approximately 10 minutes between samples.

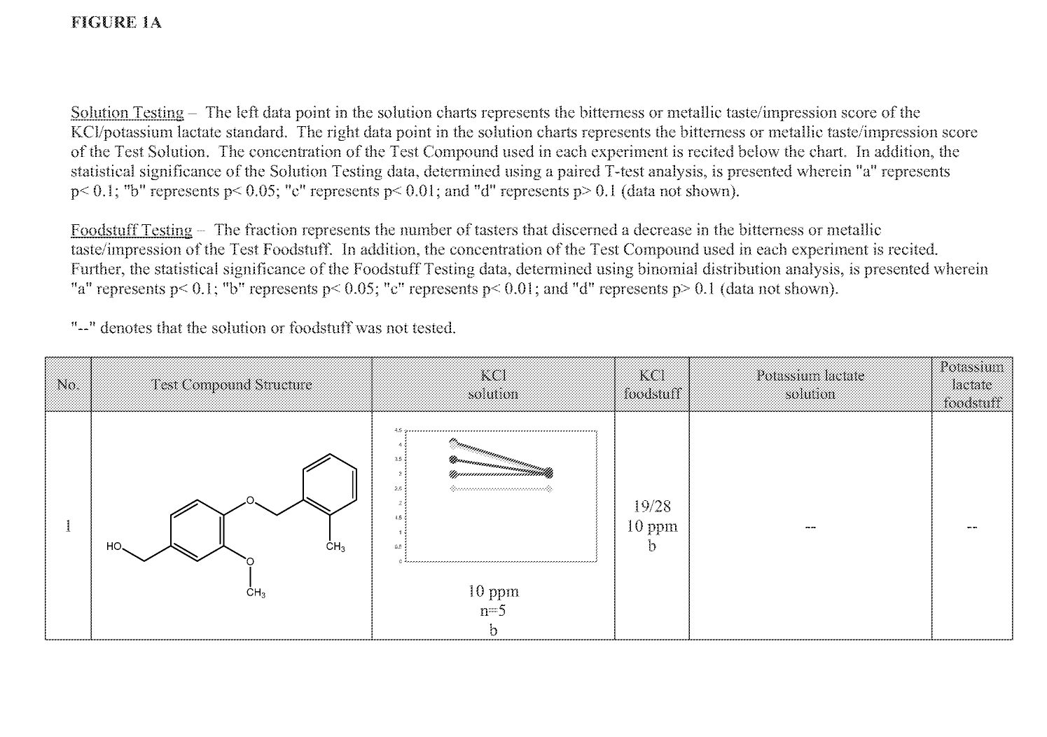

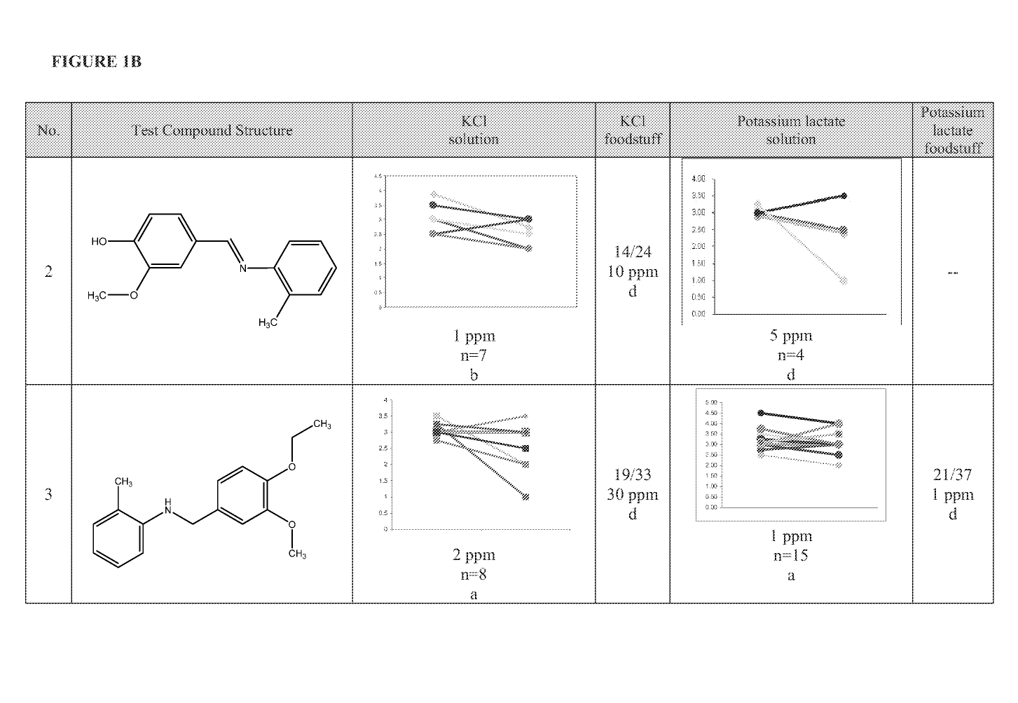

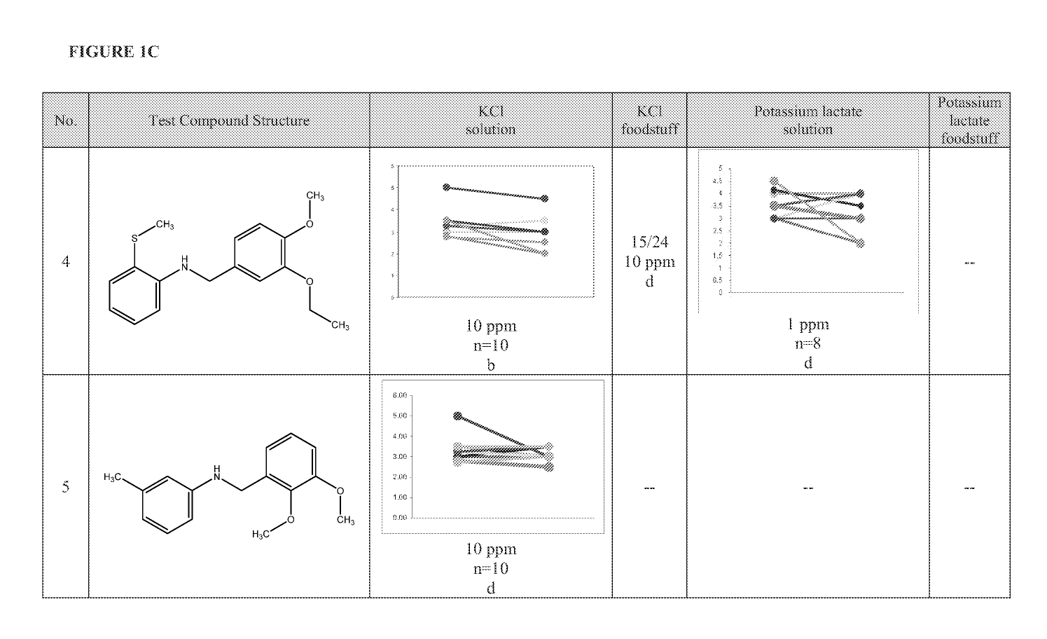

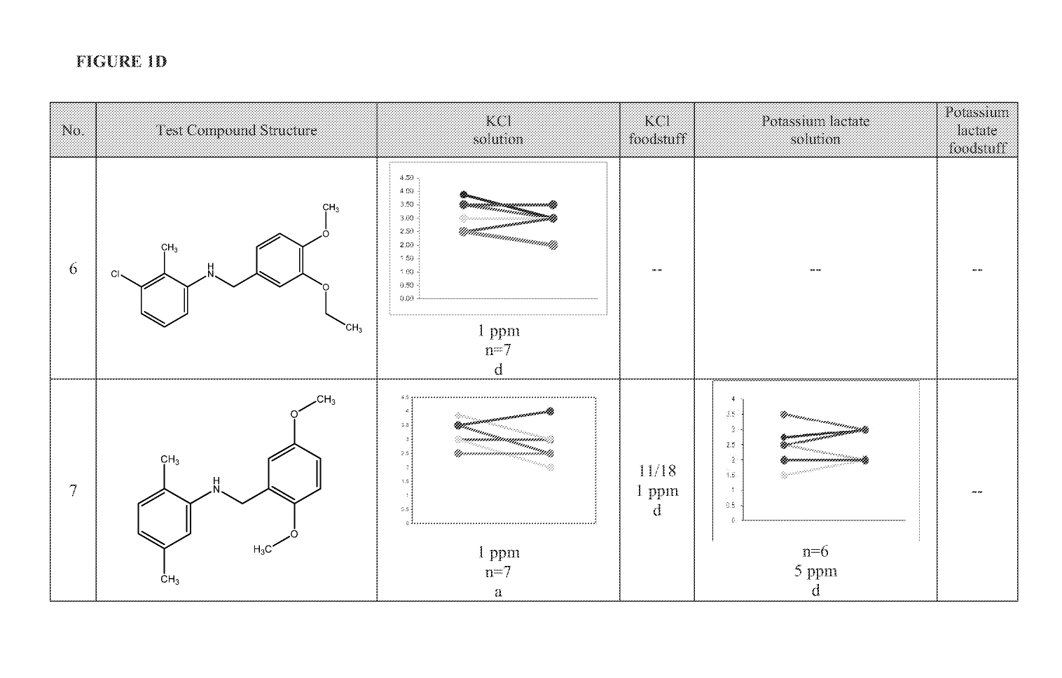

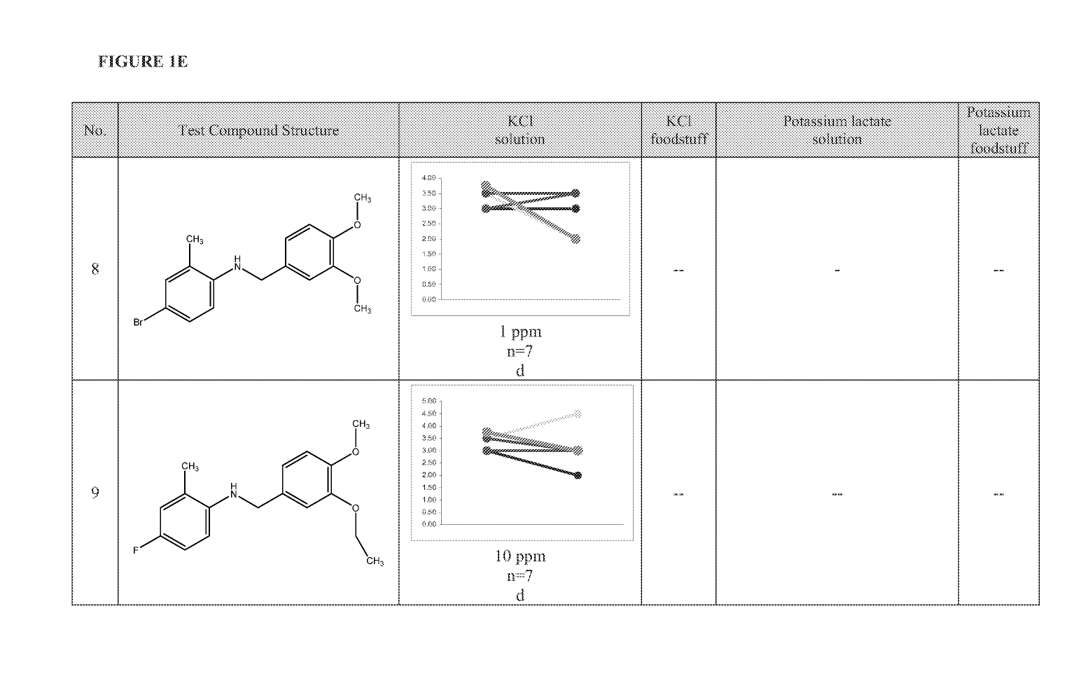

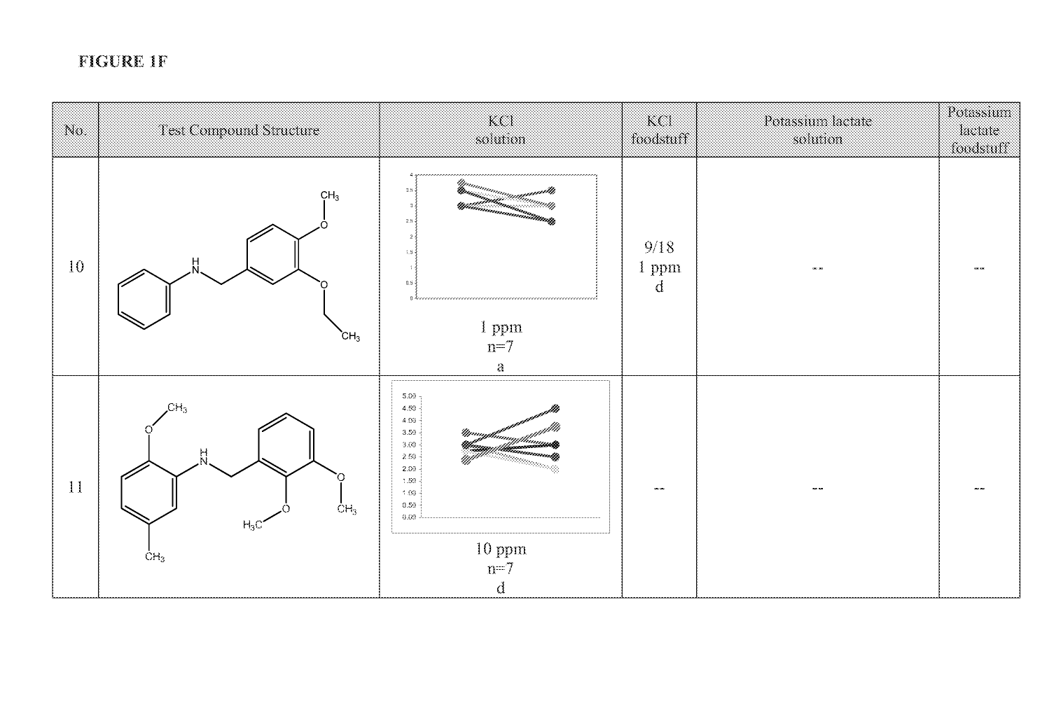

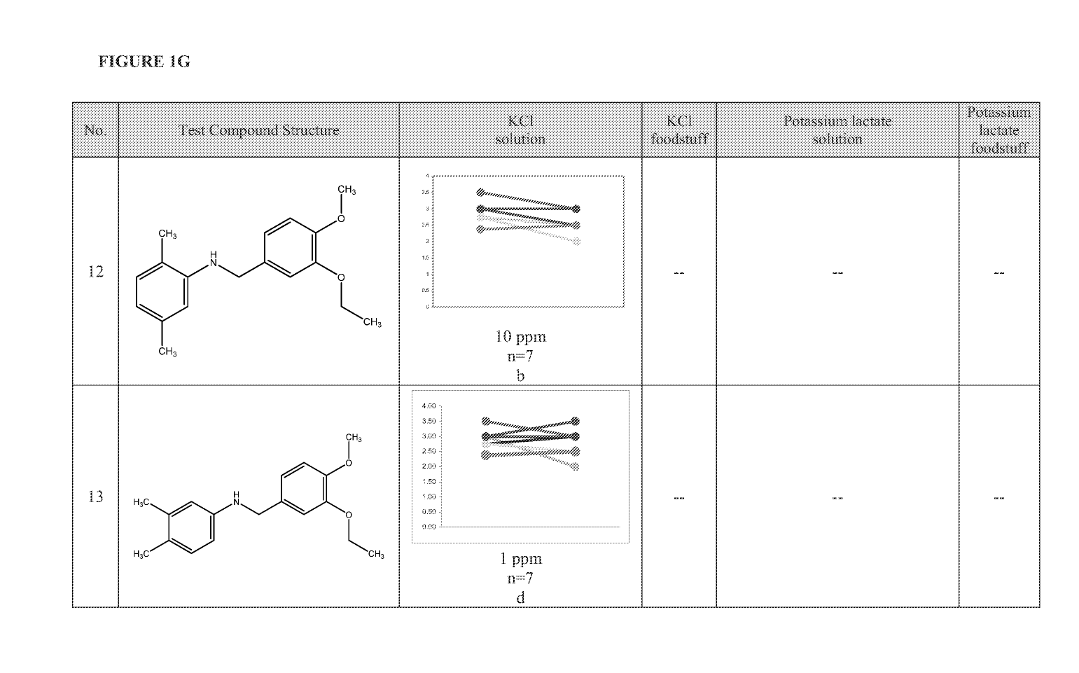

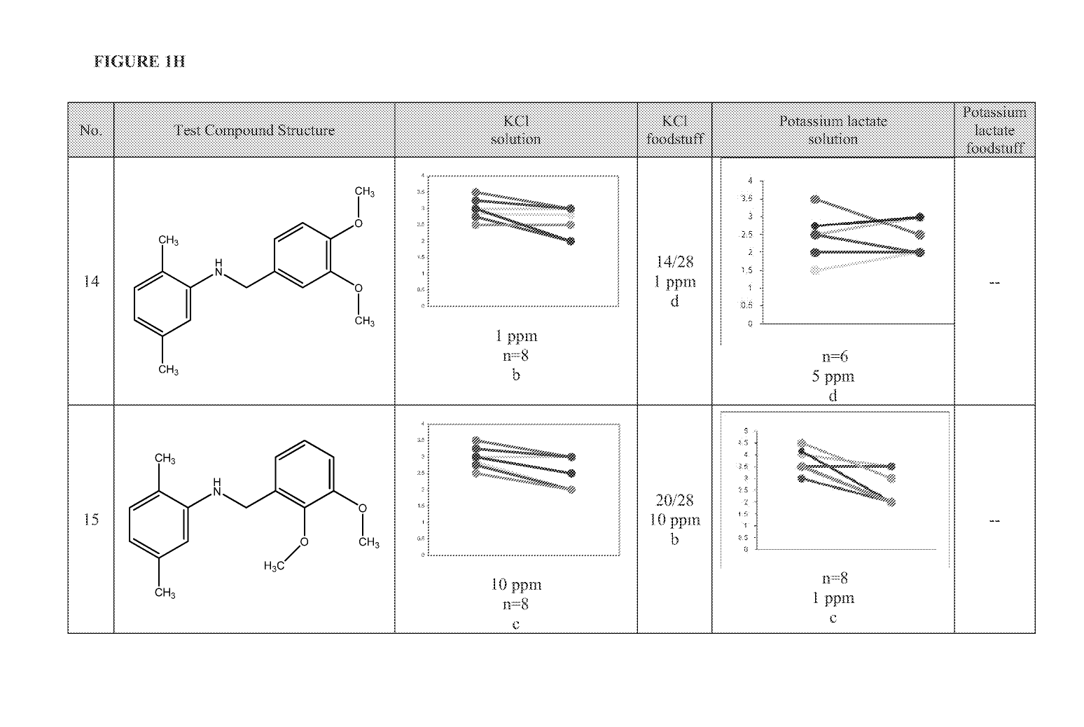

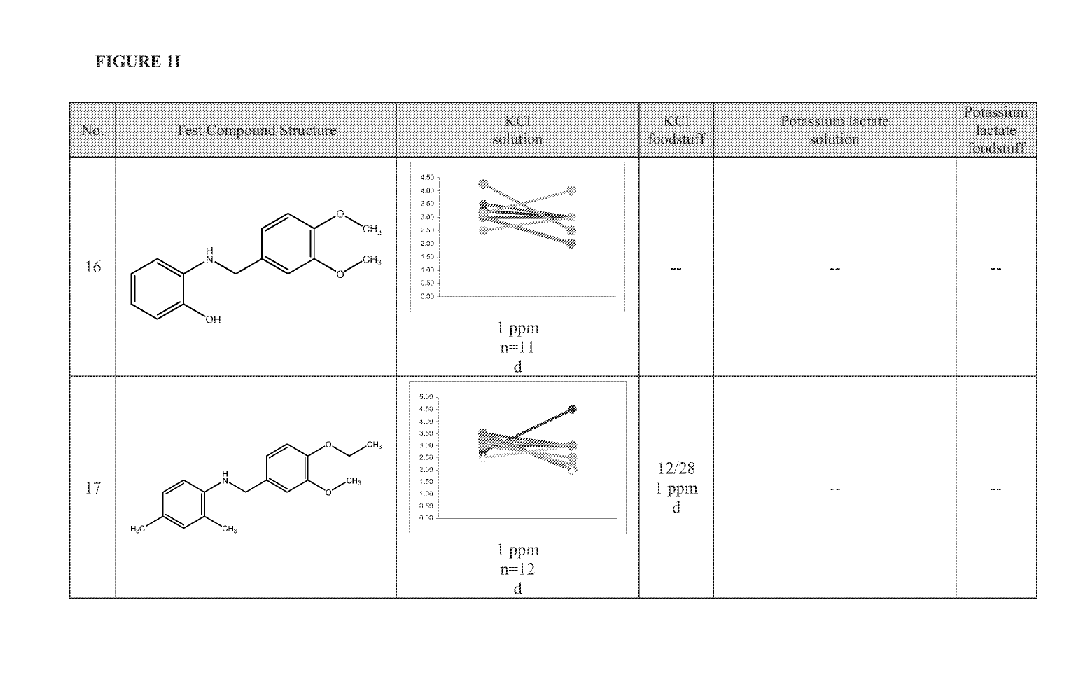

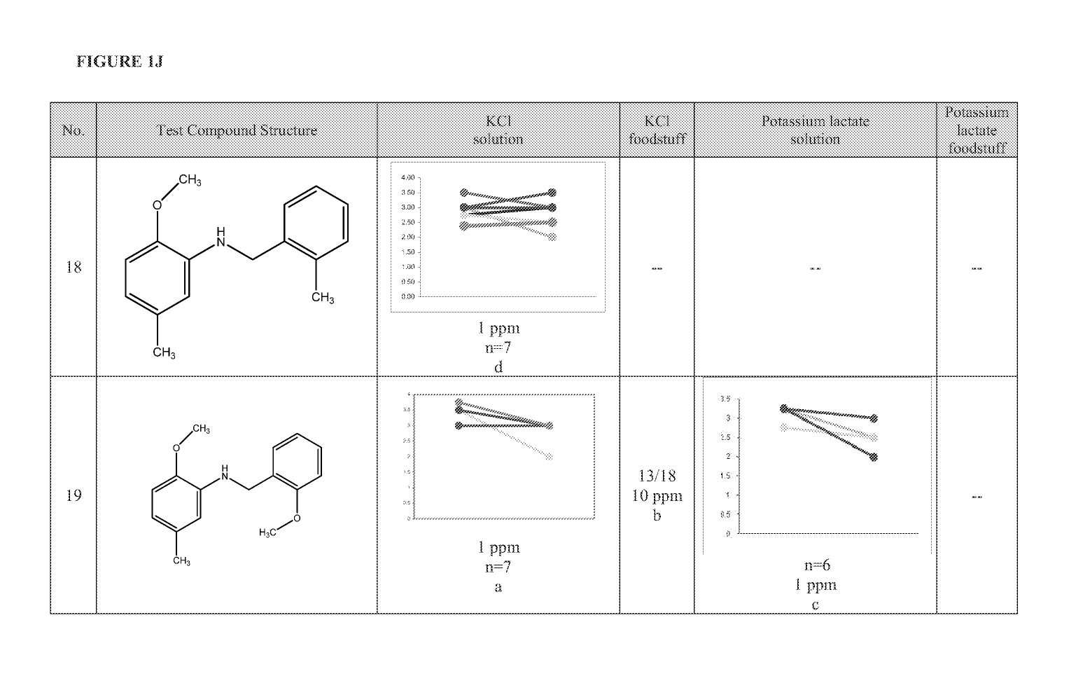

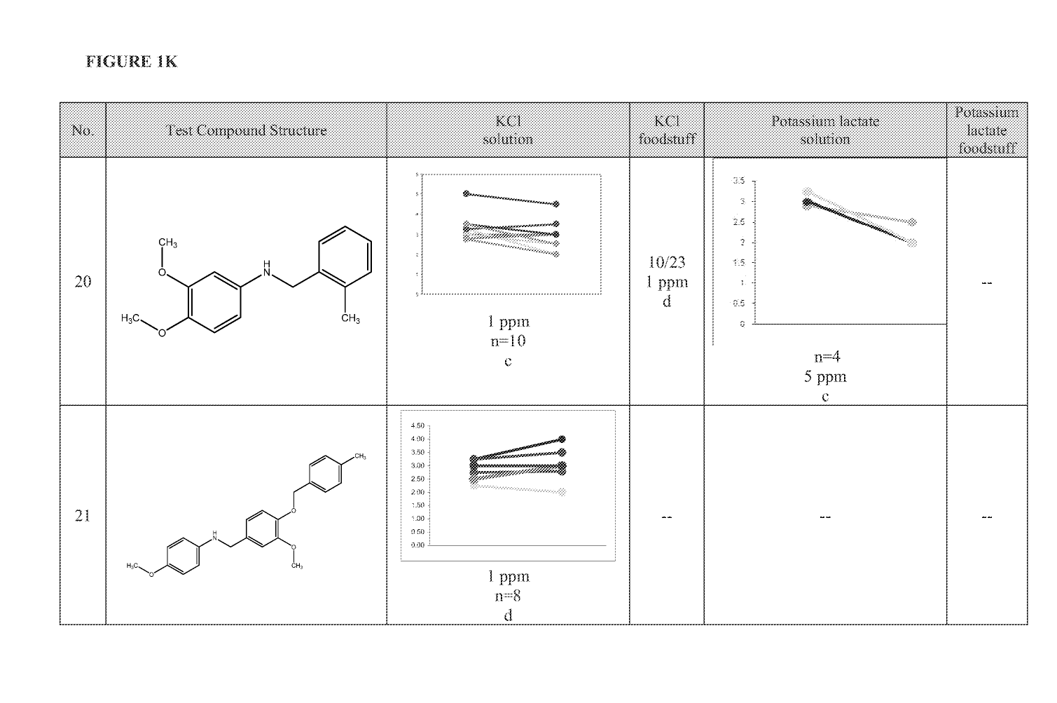

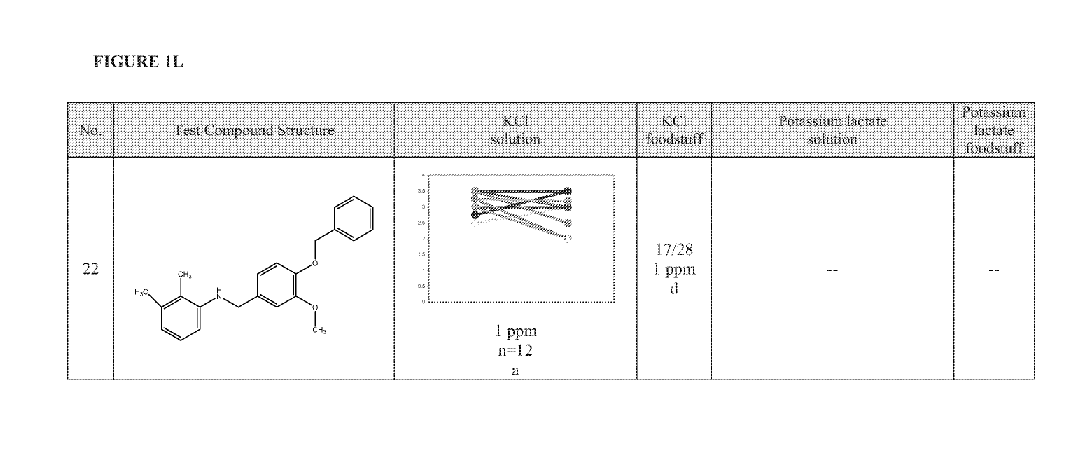

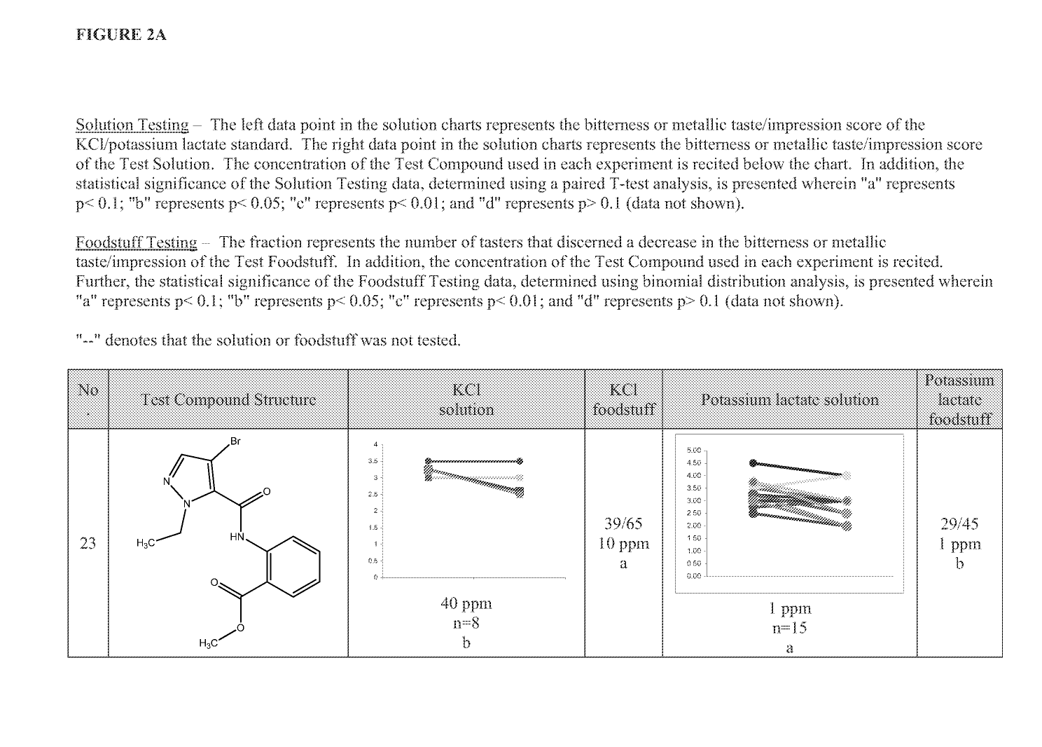

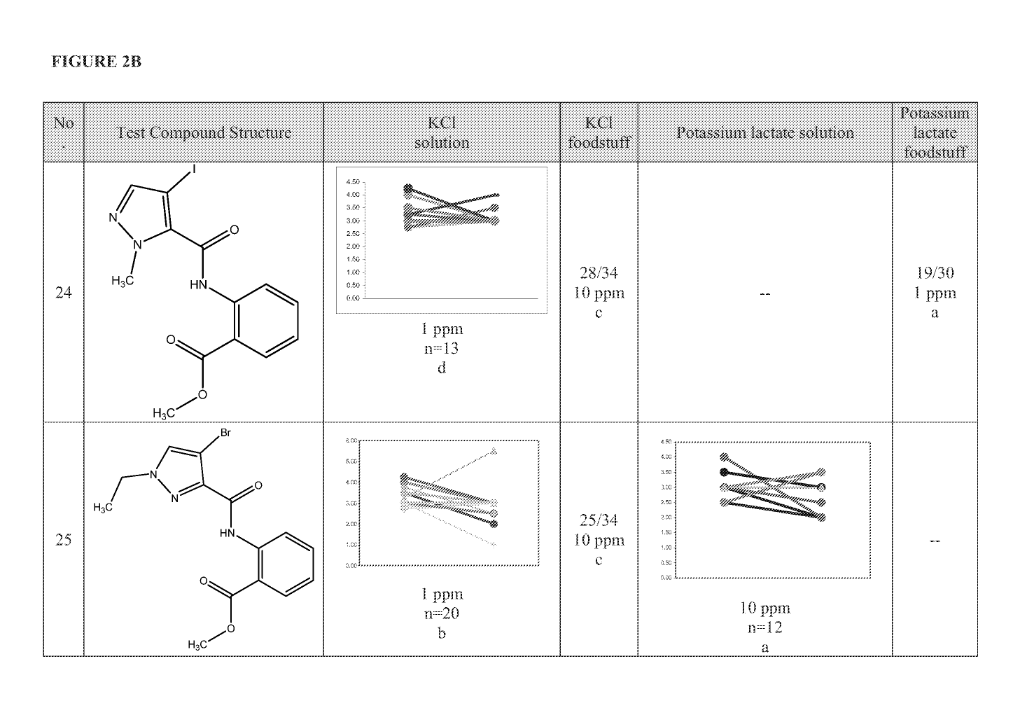

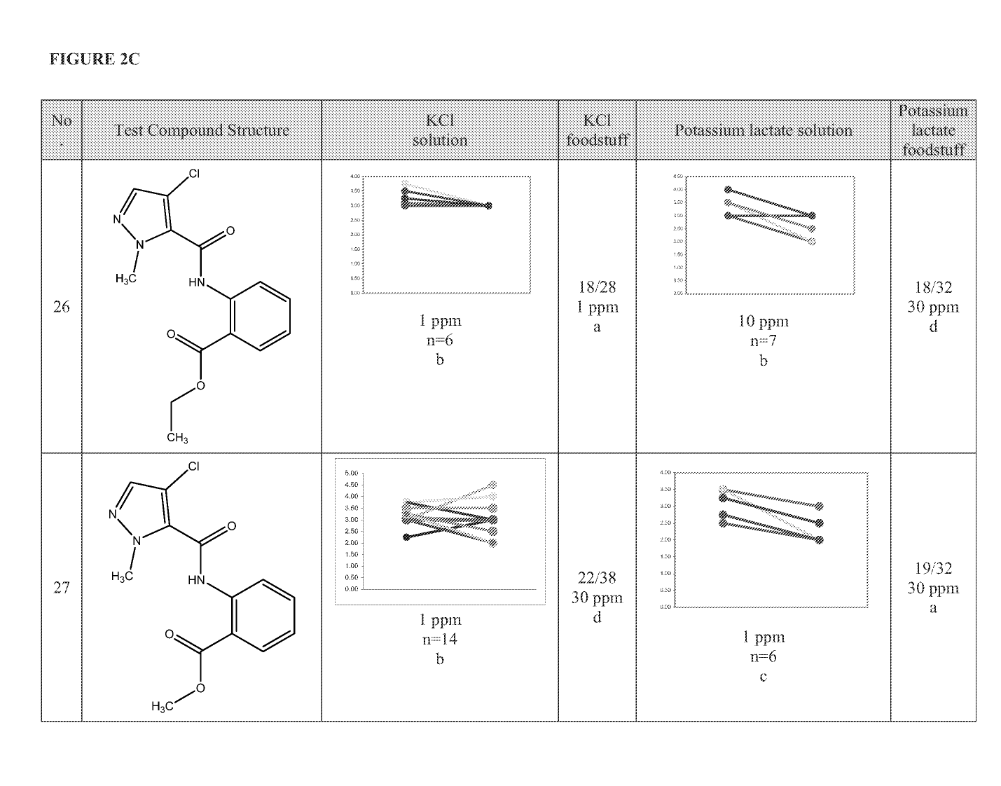

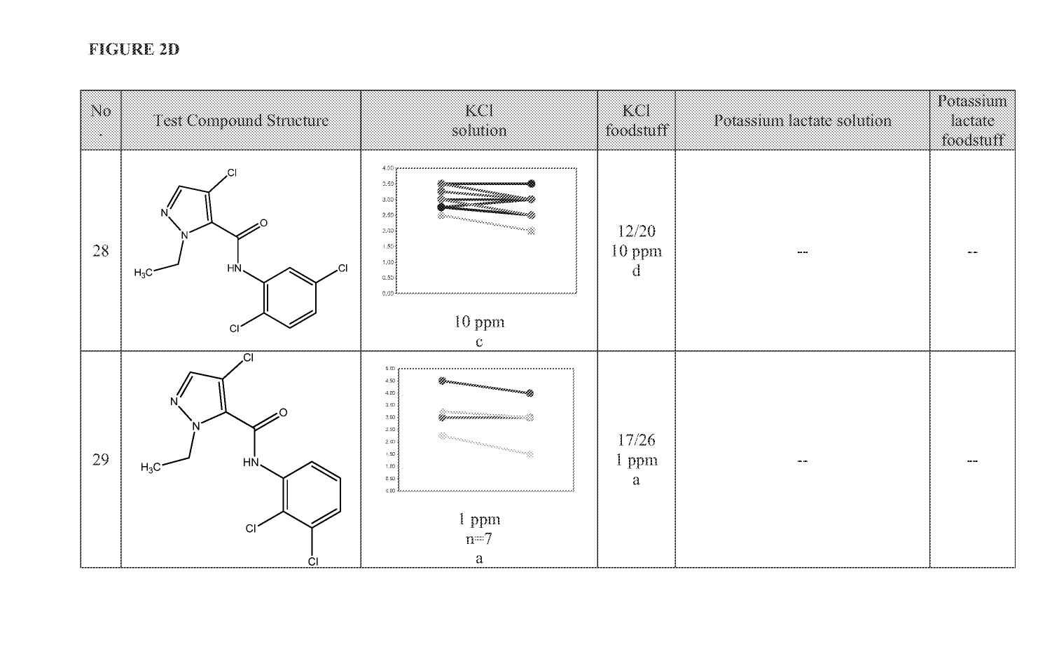

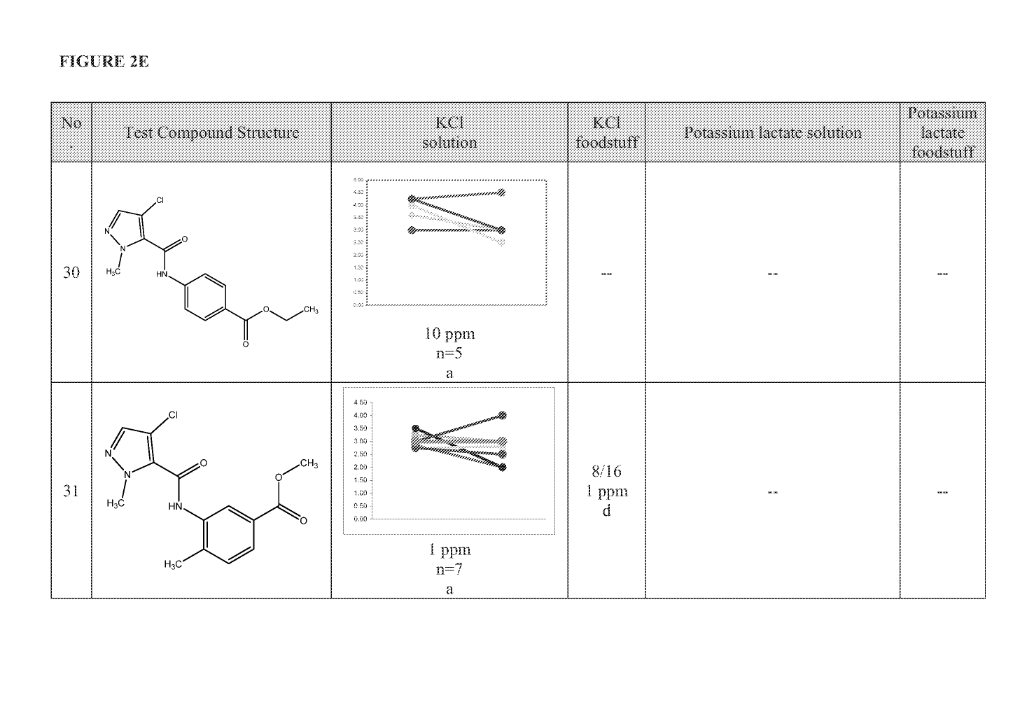

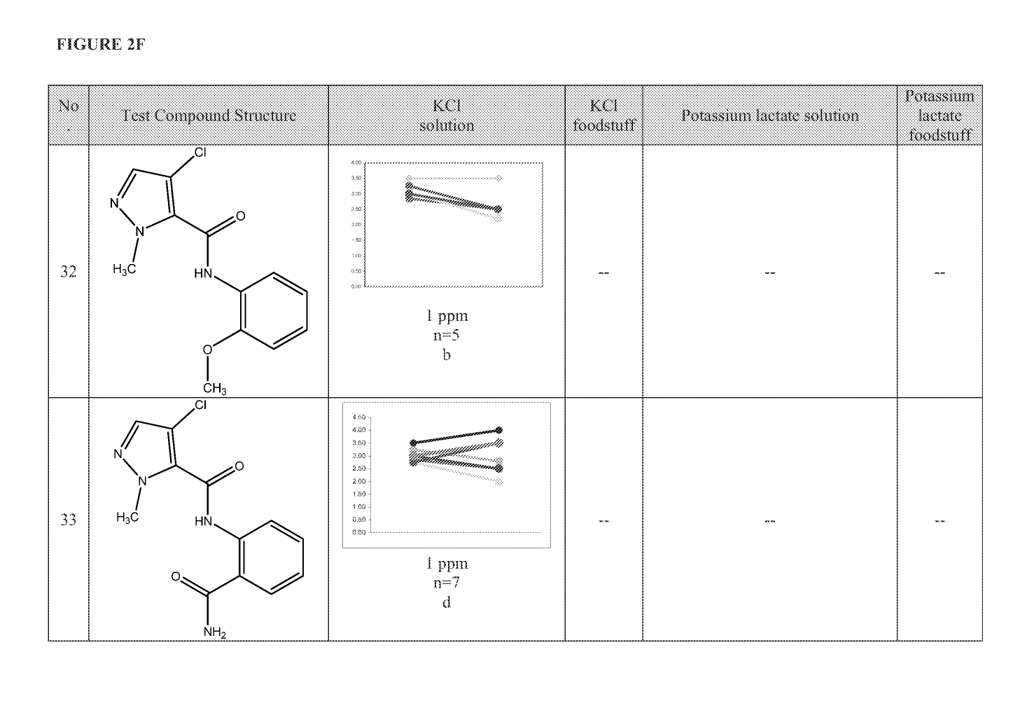

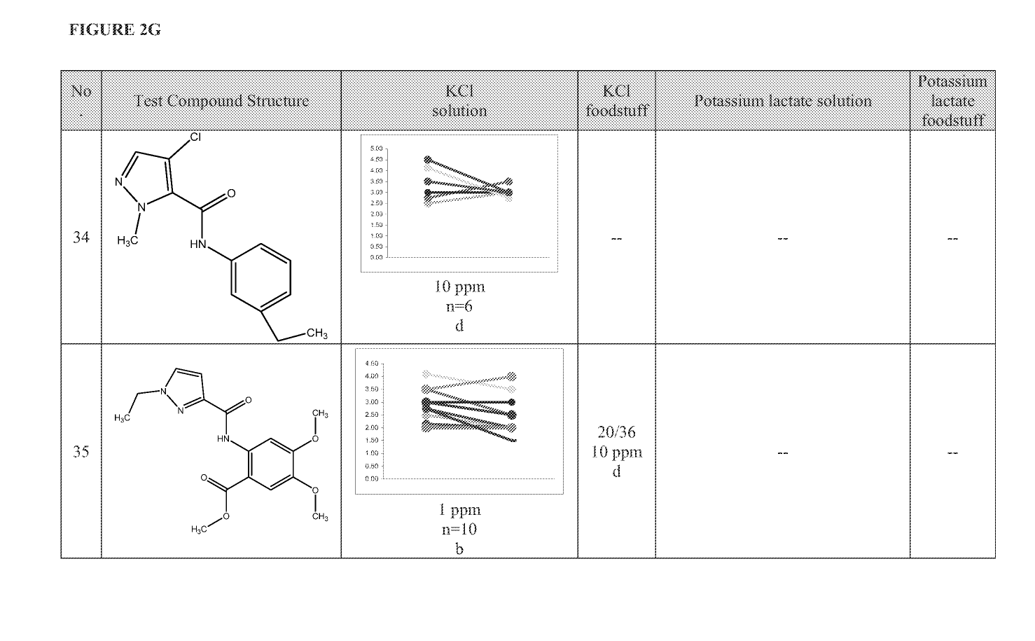

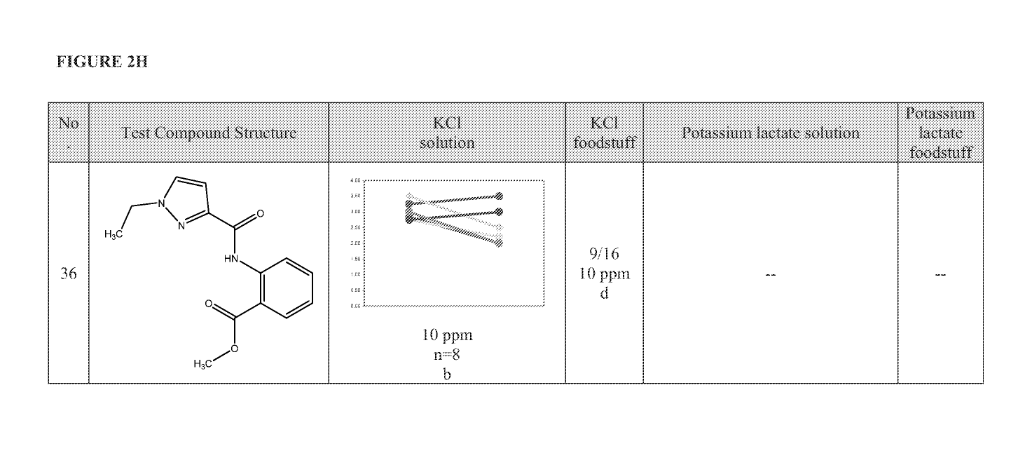

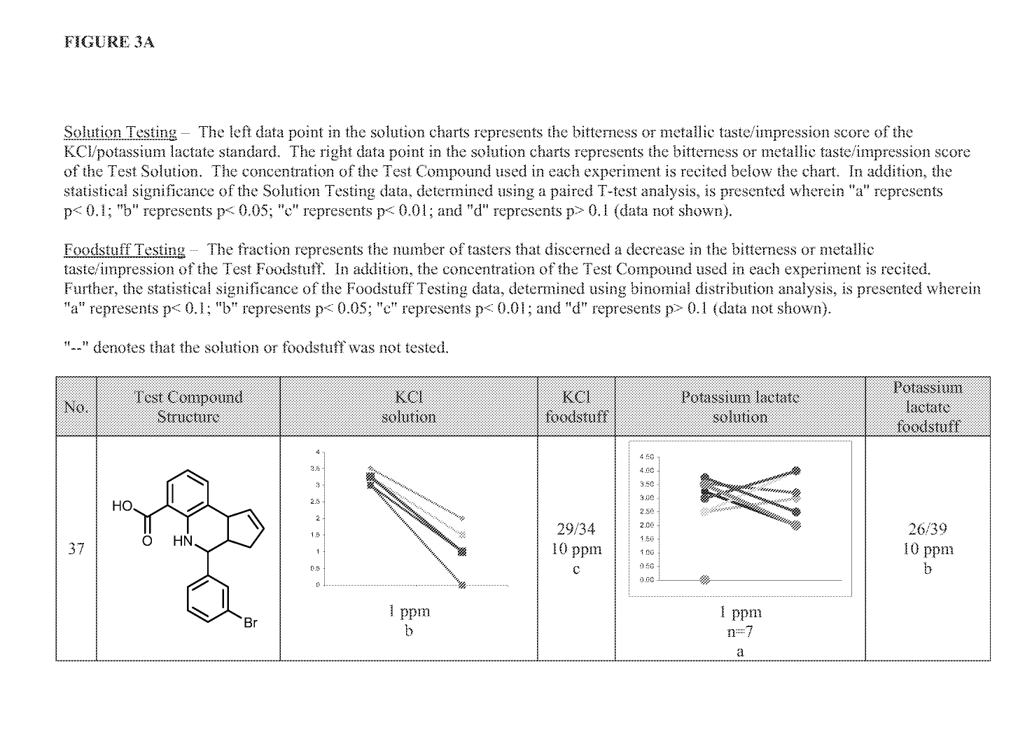

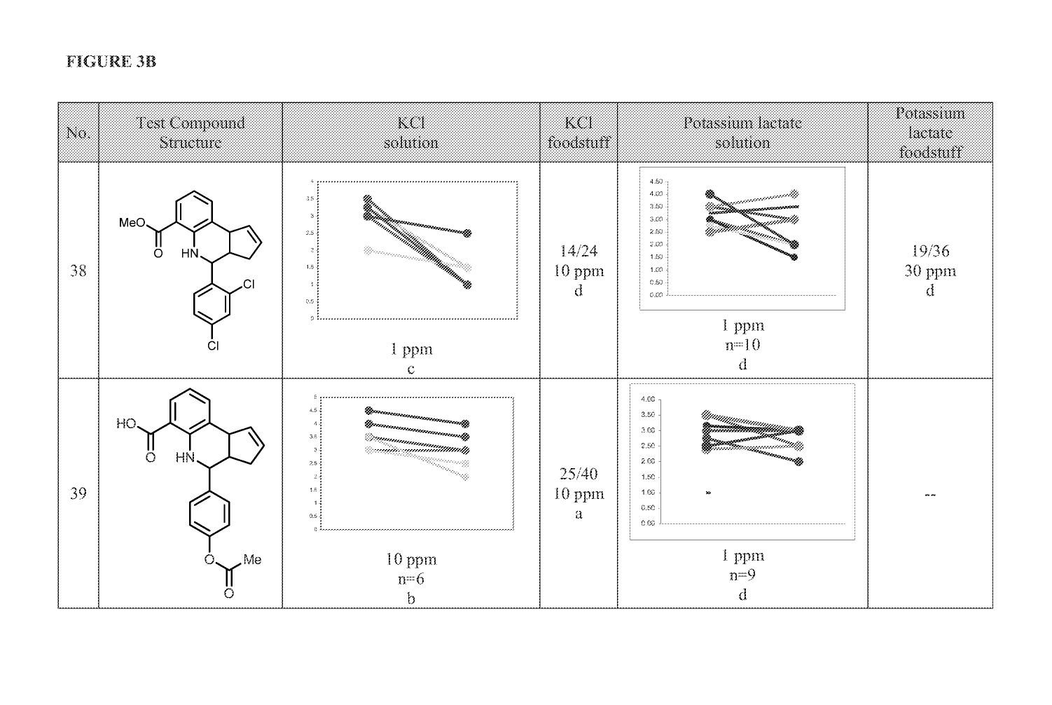

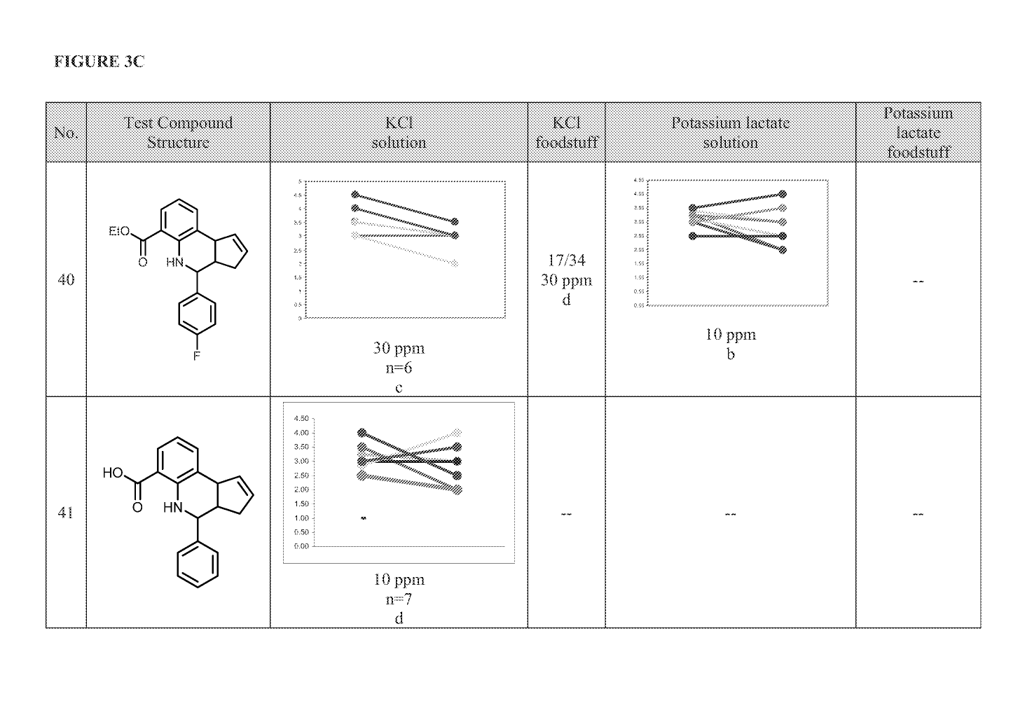

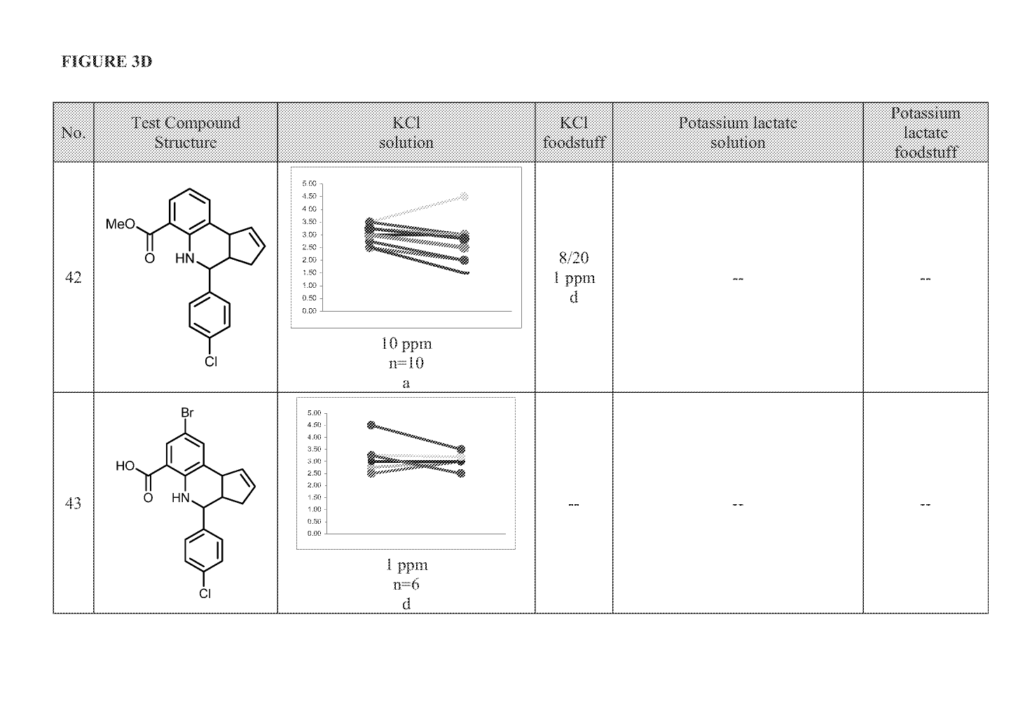

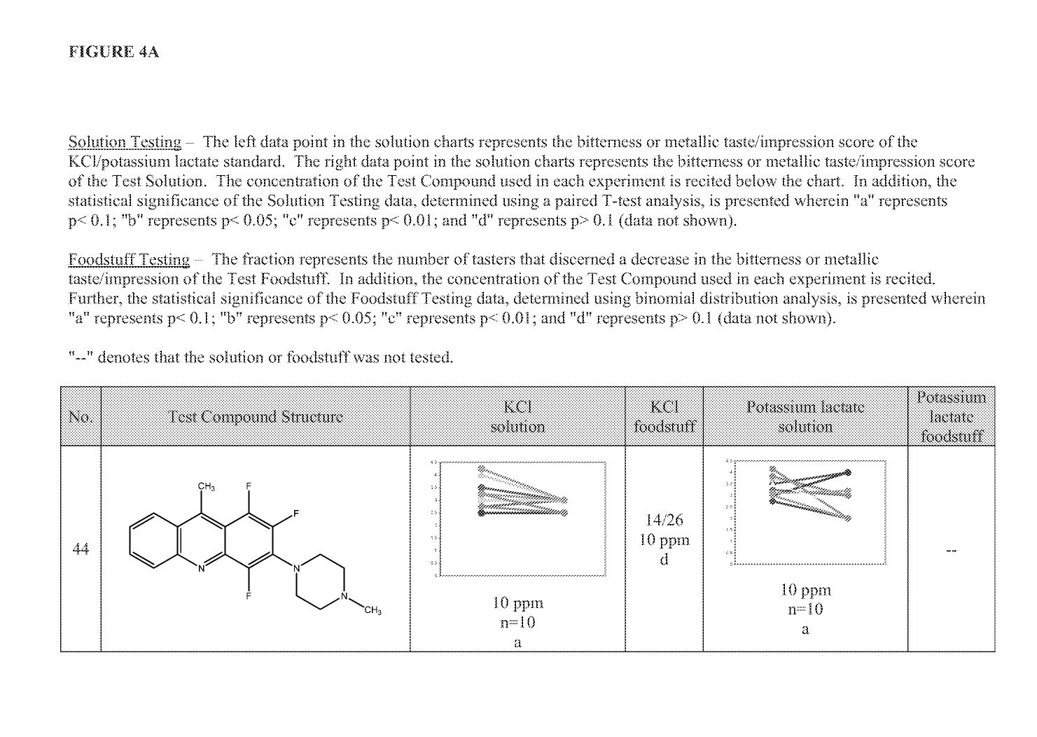

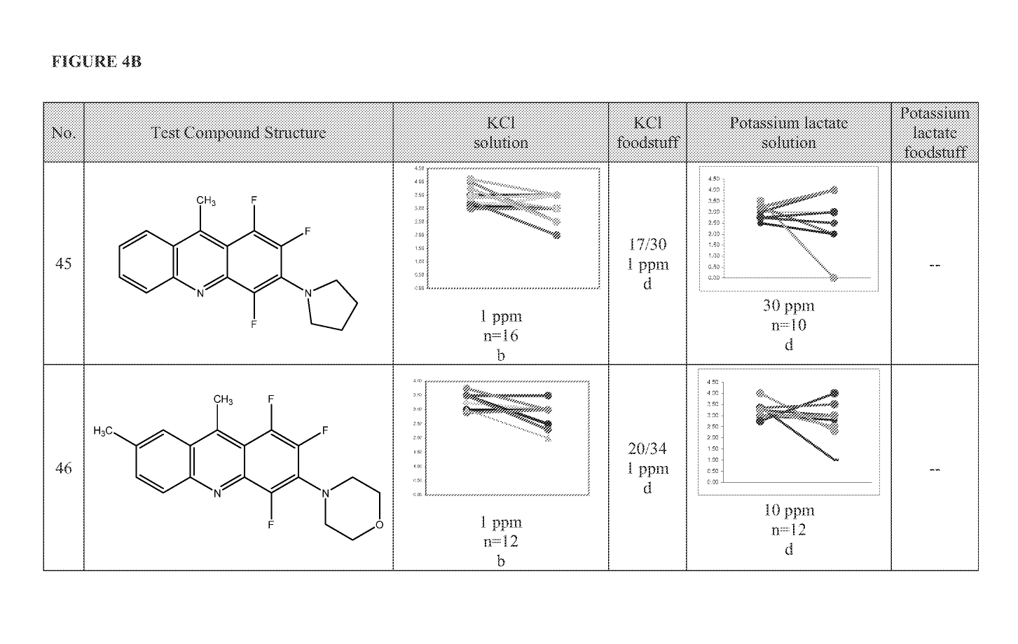

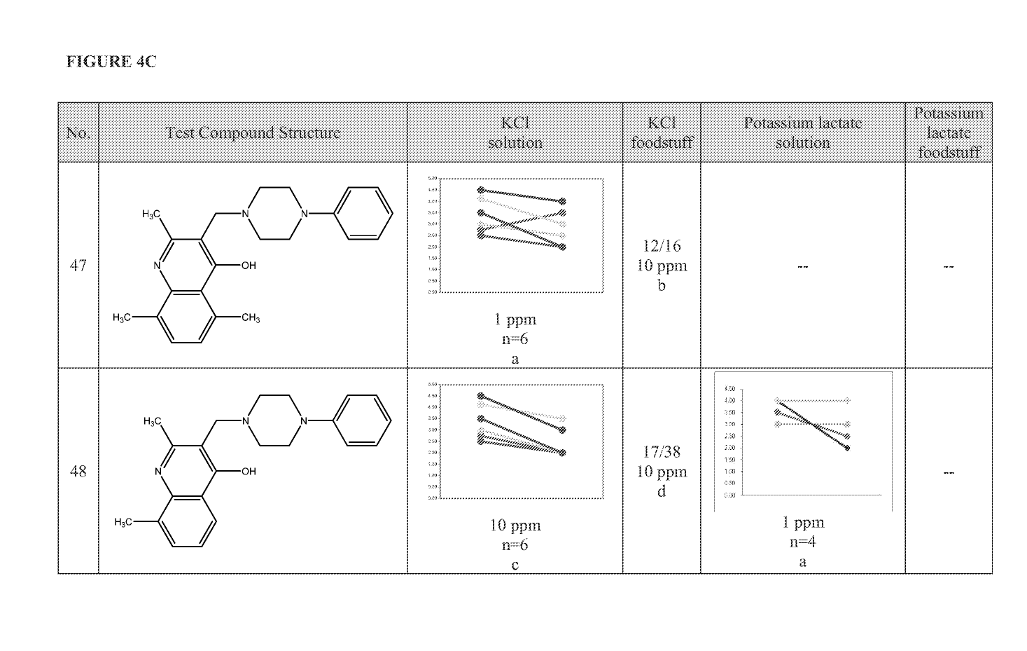

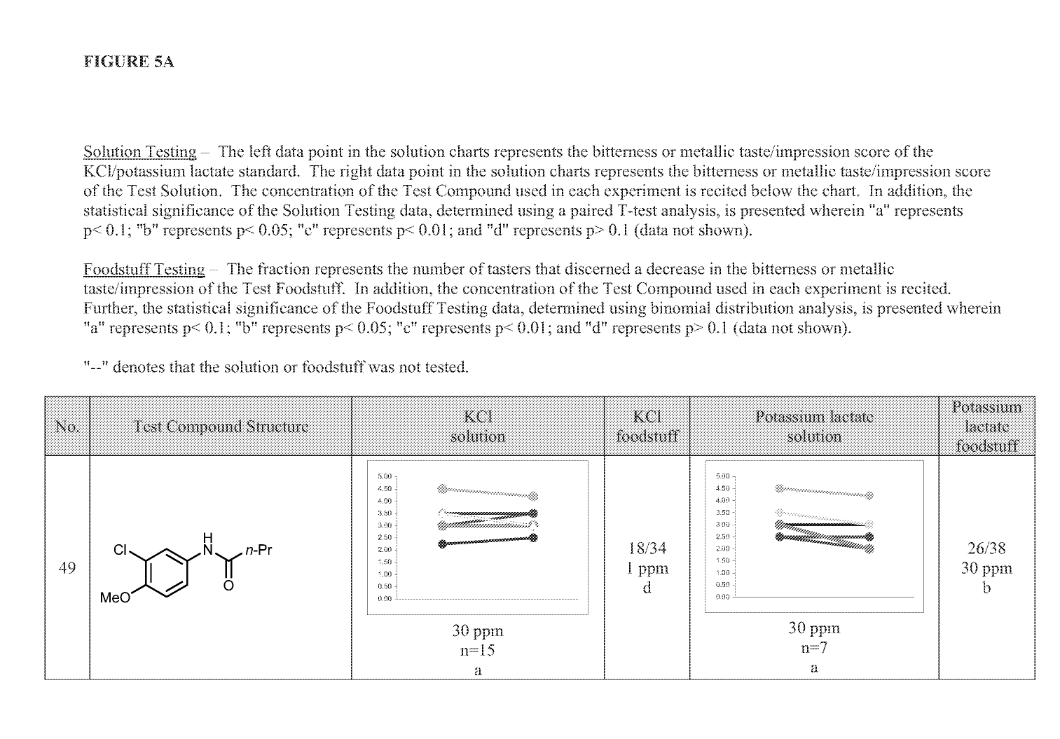

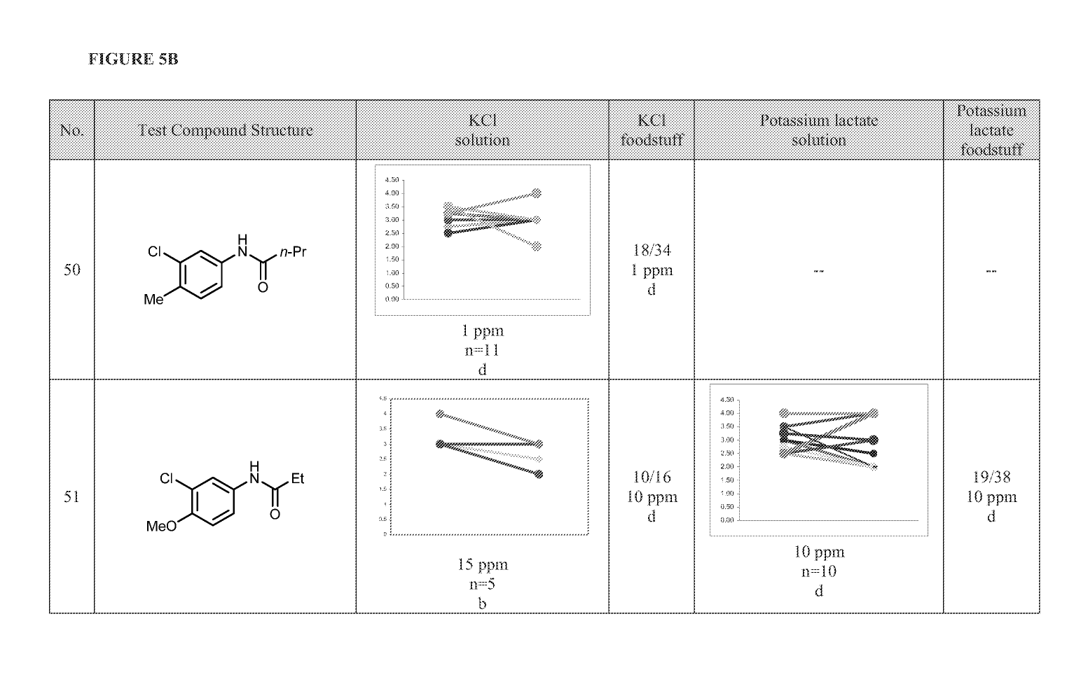

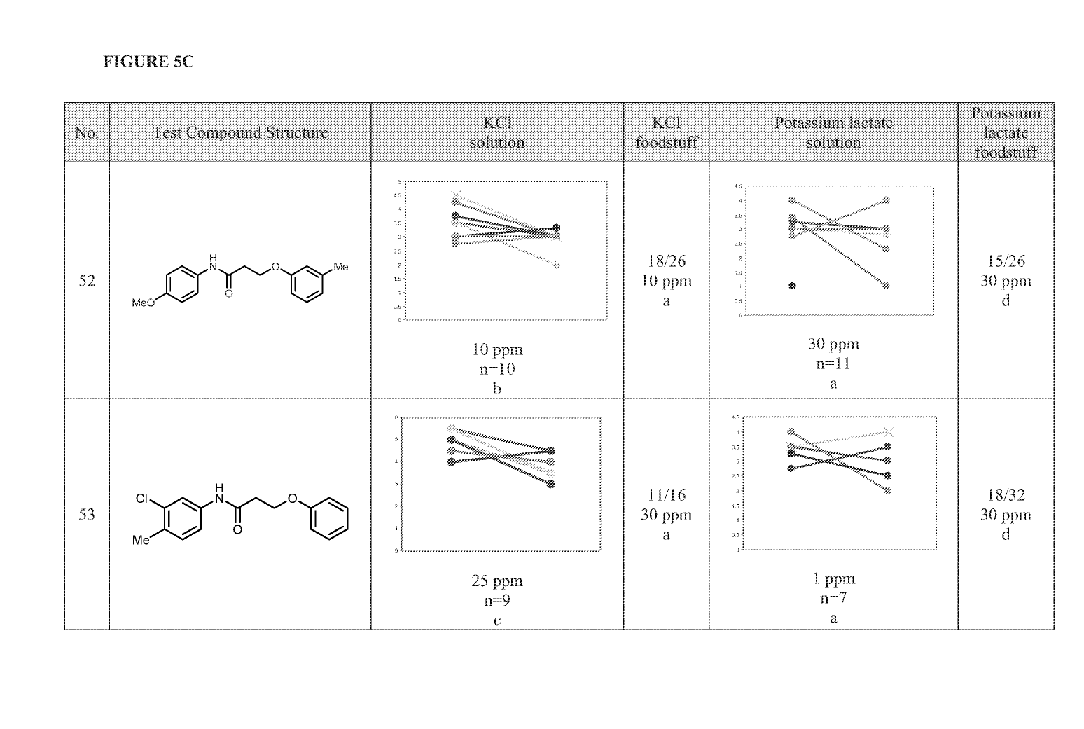

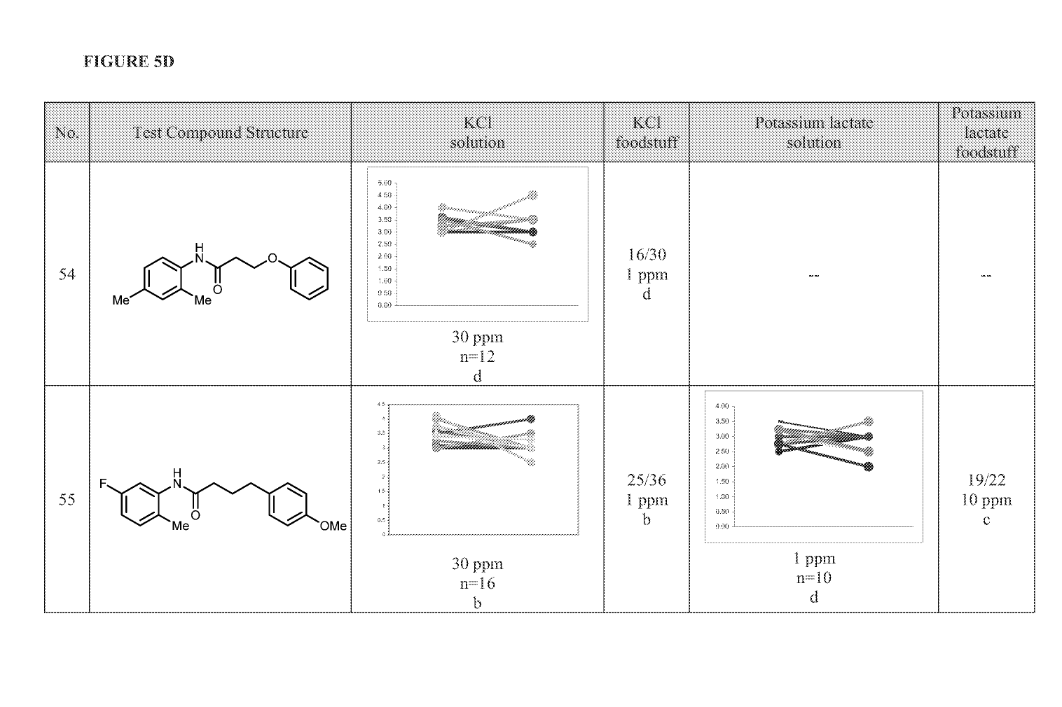

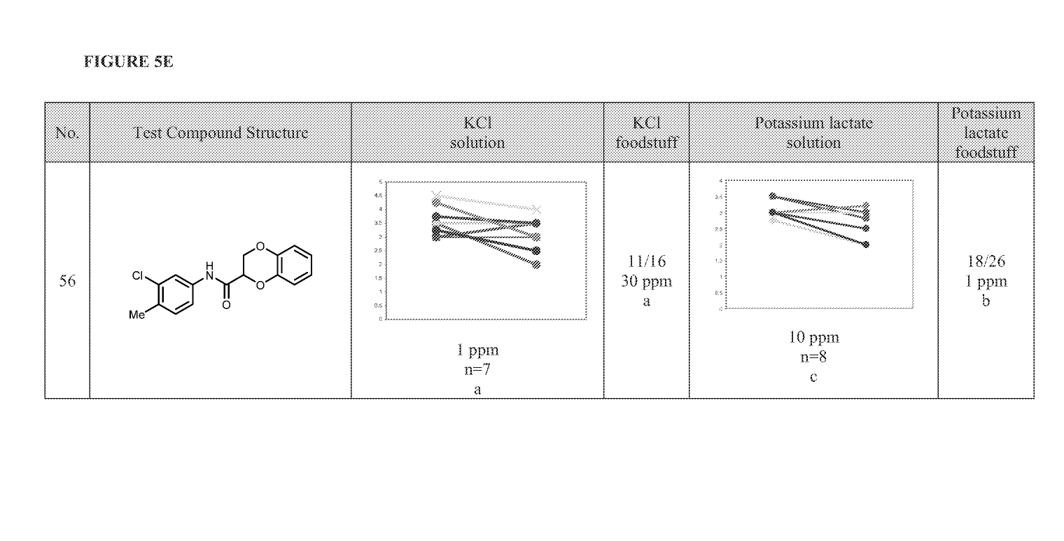

Illustrative results of the aqueous solution testing are presented in FIGS. 1-5 and Table 1.

### Example 3

### Generation of Potassium Lactate Test Solutions

Edible potassium lactate solution compositions (“potassium lactate test solutions”) were prepared by first dissolving varying amounts of the test compounds in an amount of ethanol or water (depending on the solubility of the compound) to create a 5 mg/mL stock compound solution. An amount of this stock compound solution is then added to an aqueous potassium lactate solution. Enough EtOH is then added to the resulting stock compound/potassium lactate solution so that the final potassium lactate test solution contains 1% EtOH. Potassium lactate solution standards were similarly prepared by dissolving various amounts of potassium lactate in water and ethanol without adding any test compound. Sodium lactate solution standards were similarly prepared by dissolving various amounts of sodium lactate in water and ethanol without adding any test compound (sodium lactate solution standards did not contain any potassium lactate).

TABLE 2
Potassium Lactate Taste Test Solutions

| | | | | Conc. Where |
|---|---|---|---|---|
| | | | Conc. Where | Decrease in |
| | | Conc. Of | Decrease in | Bitter Taste |
| Com- | Conc. | Compound | Bitter Taste | Discerned and |
| pound | Of | Tested | Was Discerned | p ≦ 0.1 |
| No. | KLac | (ppm) | (ppm) | (ppm) |
| 2 | 41 g/L | 1; 5 | 1; 5 | — |
| 3 | 41 g/L | 1; 10; 30 | 1; 10; 30 | 1 |
| 4 | 41 g/L | 1; 5 | 1; 5 | — |
| 7 | 41 g/L | 1; 5 | — | — |
| 10 | 41 g/L | 1; 10 | — | — |
| 14 | 41 g/L | 1; 5 | 5 | — |
| 15 | 41 g/L | 1; 10 | 1; 10 | 1; 10 |
| 17 | 41 g/L | 1; 10 | 1 | — |
| 19 | 41 g/L | 1; 10 | 1; 10 | 1; 10 |
| 20 | 41 g/L | 1; 5 | 1; 5 | 5 |
| 22 | 41 g/L | 1; 10 | — | — |
| 23 | 41 g/L | 1; 10; 30 | 1; 10; 30 | 1; 10 |
| 25 | 41 g/L | 1; 10; 30 | 10 | 10 |
| 26 | 41 g/L | 1; 10; 30 | 1; 30 | 1 |
| 27 | 41 g/L | 1; 10; 30 | 1; 10; 30 | 1; 10; 30 |
| 29 | 41 g/L | 1; 5 | 5 | — |
| 37 | 41 g/L | 0.1; 1; 10 | 0.1; 1; 10 | — |
| 38 | 41 g/L | 1; 10; 30 | 1; 10; 30 | — |
| 39 | 41 g/L | 1; 10; 30 | 1; 30 | — |
| 40 | 41 g/L | 1; 10; 30 | 1; 10; 30 | 1; 10 |
| 44 | 41 g/L | 1; 10; 30 | 1; 10; 30 | 10 |
| 45 | 41 g/L | 1; 10; 30 | 10; 30 | — |
| 46 | 41 g/L | 1; 10; 30 | 30 | — |
| 47 | 41 g/L | 1; 10 | 1; 10 | — |
| 48 | 41 g/L | 1 | 1 | 1 |
| 49 | 41 g/L | 30 | 30 | 30 |
| 51 | 41 g/L | 1; 10; 30 | 1; 10 | — |
| 52 | 41 g/L | 1; 10; 30 | 1; 30 | 30 |
| 53 | 41 g/L | 1 | 1 | 1 |
| 55 | 41 g/L | 1; 10; 30 | 1 | — |
| 56 | 41 g/L | 1; 10; 30 | 1; 10; 30 | 10 |
| Stan- | 41 g/L | — | | |
| dard | | | | |

### Example 4

### Effect of Test Compounds on the Perception of Bitter Taste of Aqueous Potassium Lactate Solutions in Humans

The effect of the test compounds on the perception of the bitter taste of an aqueous solution of potassium lactate in humans was evaluated using the “sip and spit” test described in Example 2.

Illustrative results of the aqueous solution testing are presented in FIGS. 1-5 and in Table 2.

### Example 5

### Generation of KCl Test Foodstuff Slurries

Edible KCl food compositions (“KCl test foodstuff slurries”) were prepared as follows. Dehydrated, salt-free turkey powder was weighed and mixed with various amounts of KCl and/or NaCl and then solubilized with boiling water to create a homogenized solubilized turkey slurry. Varying amounts of the test compounds were dissolved in an amount of ethanol or water (depending on the solubility of the compound) to create a 5 mg/mL stock compound solution. An amount of this stock compound solution was then added to the turkey slurry. Enough EtOH is then added to the resulting stock compound/turkey slurry so that the slurry contains 1% EtOH. The slurry was again homogenized by boiling and mixing and allowed to cool to yield the final KCl test foodstuff slurry for taste testing. KCl foodstuff slurry standards were similarly prepared without any test compound. NaCl foodstuff slurry standards were similarly prepared without adding any test compound (NaCl foodstuff slurry standards did not contain any KCl).

TABLE 3
KCl Foodstuff Slurry Compositions

| | | | | Conc. at |
|---|---|---|---|---|
| | | | Conc. at | Which At Least |
| | | | Which At Least | 50% of Panelist |
| | | Conc. of | 50% of Panelist | Discerned Decrease |
| Com- | Conc. | Compound | Discerned Decrease | in Bitter Taste |
| pound | Of | Tested | in Bitter Taste | and p ≦ 0.1 |
| No. | KCl | (ppm) | (ppm) | (ppm) |
| 1 | 1.6% | 1 | 1 | — |
| 2 | 1.6% | 1; 10 | 10 | — |
| 3 | 1.6% | 1; 10; 30 | 1; 10; 30 | — |
| 4 | 1.6% | 1; 10 | 1; 10 | — |
| 7 | 1.6% | 1; 10 | 1 | — |
| 10 | 1.6% | 1; 10 | — | — |
| 12 | 1.6% | 1; 10 | 10 | — |
| 14 | 1.6% | 1; 10 | — | — |
| 15 | 1.6% | 1; 10 | 10 | 10 |
| 17 | 1.6% | 1; 10 | — | — |
| 19 | 1.6% | 1; 10 | 1; 10 | 10 |
| 20 | 1.6% | 1; 10 | — | — |
| 22 | 1.6% | 1; 10 | 1; 10 | — |
| 23 | 1.6% | 1; 10; 30 | 1; 10 | 1; 10 |
| 24 | 1.6% | 1; 10; 30 | 1; 10 | 10 |
| 25 | 1.6% | 1; 10; 30 | 10; 30 | 10; 30 |
| 26 | 1.6% | 1; 10; 30 | 1; 10 | 1 |
| 27 | 1.6% | 1; 10; 30 | 30 | — |
| 28 | 1.6% | 1; 10 | 10 | — |
| 29 | 1.6% | 1 | 1 | 1 |
| 31 | 1.6% | 1 | 1 | — |
| 35 | 1.6% | 1; 10; 30 | 10 | — |
| 36 | 1.6% | 10 | 10 | — |
| 37 | 1.6% | 1; 10; 30 | 1; 10; 30 | 10; 30 |
| 38 | 1.6% | 1; 10; 30 | 1; 10; 30 | — |
| 39 | 1.6% | 1; 10; 30 | 1; 10 | 10 |
| 40 | 1.6% | 1; 10; 30 | | — |
| 42 | 1.6% | 1; 10; 30 | 1 | — |
| 44 | 1.6% | 10 | 10 | — |
| 45 | 1.6% | 0.1; 1; 10 | 1 | — |
| 46 | 1.6% | 1; 10; 30 | 1 | — |
| 47 | 1.6% | 10 | 10 | 10 |
| 48 | 1.6% | 10 | 10 | — |
| 49 | 1.6% | 1 | 1 | 1 |
| 50 | 1.6% | 1; 10; 30 | 1; 30 | — |
| 51 | 1.6% | 1; 10; 30 | 1; 10 | — |
| 52 | 1.6% | 1; 10; 30 | 10; 30 | 10 |
| 53 | 1.6% | 1; 10; 30 | 30 | 30 |
| 54 | 1.6% | 1; 10; 30 | 1 | — |
| 55 | 1.6% | 1; 10; 30 | 1; 10 | 1; 10 |
| 56 | 1.6% | 1; 10; 30 | 10; 30 | — |
| Stan- | 1.6% | — | | |
| dard | | | | |

### Example 6

### Effect of Test Compounds on the Perception of Bitter Taste of KCl Foodstuff Slurries in Humans Using a Two-Alternative Forced Choice Method (2AFC)

The effect of the test compounds on the perception of the bitter taste of KCl foodstuff slurries in humans was evaluated using a two-alternative-forced-choice “sip and spit” test as follows.

In a blind taste test, panelists received two portions of turkey slurry—one portion being the KCl foodstuff slurry standard and the other being one of the KCl test foodstuff slurries (each prepared as described in Example 5). The panelists tasted each of the portions by sipping and spitting. Each sample was tested in 2-4 discrete taste test experiments. Panelists were asked to rinse with water, eat a cracker, and wait about 10 minutes between samples. In each case, the panelists were asked to compare the bitter taste of the two turkey samples to each other (i.e. panelists were asked to indicate which sample was less bitter).

Illustrative results of the foodstuff testing are presented in FIGS. 1-5 and in Table 3.

### Example 7

### Generation of Potassium Lactate Test Foodstuff Slurries

Edible potassium lactate food compositions (“potassium lactate test foodstuff slurries”) were prepared as follows, Dehydrated, salt-free turkey powder was weighed and mixed with various amounts of potassium lactate and/or sodium lactate and then solubilized with boiling water to create a homogenized solubilized turkey slurry. Varying amounts of the test compounds were dissolved in an amount of ethanol or water (depending on the solubility of the compound) to create a 5 mg/mL stock compound solution. An amount of this stock compound solution was then added to the turkey slurry. Enough EtOH is then added to the resulting stock compound/turkey slurry so that the final slurry contains 1% EtOH. The final slurry was again homogenized by boiling and mixing and allowed to cool to yield the final slurry for taste testing. Potassium lactate foodstuff slurry standards were similarly prepared without any test compound. Sodium lactate foodstuff slurry standards were similarly prepared without adding any test compound (sodium lactate foodstuff slurry standards did not contain any potassium lactate).

TABLE 4
Potassium Lactate Foodstuff Slurry Compositions

| | | | | Conc. at |
|---|---|---|---|---|
| | | | Conc. at | Which At Least |
| | | | Which At Least | 50% of Panelist |
| | | Conc. of | 50% of Panelist | Discerned Decrease |
| Com- | Conc. | Compound | Discerned Decrease | in Bitter Taste |
| pound | Of | Tested | in Bitter Taste | and p ≦ 0.1 |
| No. | KLac | (ppm) | (ppm) | (ppm) |
| 3 | 4.5% | 1; 10; 30 | 1; 10; 30 | — |
| 23 | 4.5% | 1; 10; 30 | 1; 30 | 1 |
| 24 | 4.5% | 1; 10; 30 | 1; 10; 30 | 30 |
| 25 | 4.5% | 1; 10; 30 | 1 | — |
| 26 | 4.5% | 1; 10; 30 | 30 | — |
| 27 | 4.5% | 1; 10; 30 | 1; 30 | — |
| 37 | 4.5% | 1; 10; 30 | 1; 10; 30 | 10 |
| 38 | 4.5% | 1; 10; 30 | 30 | — |
| 49 | 4.5% | 30 | 30 | 30 |
| 51 | 4.5% | 1; 10; 30 | 10 | — |
| 52 | 4.5% | 1; 10; 30 | 10; 30 | — |
| 53 | 4.5% | 30 | 30 | — |
| 55 | 4.5% | 1; 10; 30 | 10 | — |
| 56 | 4.5% | 1; 10; 30 | 1 | 1 |
| Stan- | 4.5% | — | | |
| dard | | | | |

### Example 8

### Effect of Test Compounds on the Perception of Bitter Taste of Potassium Lactate Foodstuff Slurries in Humans Using a Two-Alternative Forced Choice Method (2AFC)

The effect of the test compounds on the perception of the bitter taste of potassium lactate foodstuffs in humans was evaluated using the two-alter native-forced-choice “sip and spit” test described in Example 6.

Illustrative results of the foodstuff testing are presented in FIGS. 1-5 and in Table 4.

1. A composition comprising a compound according to a formula selected from the group consisting of:
(a) Formula (I): 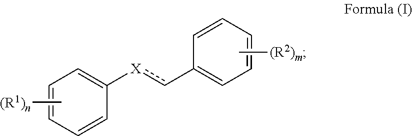
or a comestibly or biologically acceptable salt or derivative thereof,
wherein, as valence and stability permit:
R¹, independently for each occurrence, is selected from the group consisting of halo; hydroxyl; C₁₋₆alkyl; C₁₋₆haloalkyl, C₁₋₆hydroxylalkyl, or C₁₋₆acyloxy-C₁₋₆alkyl; C₂₋₆alkenyl; C₂₋₆alkynyl; C₁₋₆alkoxy; C₁₋₆alkylthio; and C₆₋₁₀aryl-C₁₋₆alkyloxy optionally substituted with halo, hydroxyl, C₁₋₆alkyl, C₁₋₆alkoxy, or C₁₋₆acyloxy;
R², independently for each occurrence, is selected from the group consisting of halo; hydroxyl; C₁₋₆alkyl; C₁₋₆haloalkyl, C₁₋₆hydroxylalkyl, or C₁₋₆acyloxy-C₁₋₆alkyl; C₂₋₆alkenyl; C₂₋₆alkynyl; C₁₋₆alkoxy; C₁₋₆alkylthio; and C₆₋₁₀aryl-C₁₋₆alkyloxy optionally substituted with halo, hydroxyl, C₁₋₆alkyl, C₁₋₆alkoxy, or C₁₋₆acyloxy;
X is O or NR^{a}, wherein R^{a} is absent or is selected from the group consisting of hydrogen and C₁₋₆alkyl;
wherein any of R¹, R², and R^{a}, independently and independently for each occurrence, is optionally substituted with 1-3 substituents selected from the group consisting of C₁₋₁₀alkyl, C₁₋₁₀haloalkyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl; and wherein heterocyclic or heteroaromatic rings, independently for each occurrence, comprise 1-4 heteroatoms selected from N, O, and S;
m is 1-3;
n is 0-3; and
wherein the composition is edible and capable of reducing bitter taste of a bitter tastant
(b) Formula (IV): 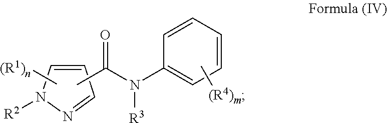
or a comestibly or biologically acceptable salt or derivative thereof,
wherein, as valence and stability permit:
R¹, independently for each occurrence, is selected from the group consisting of halo, C₁₋₆alkyl, C₂₋₆alkenyl, and C₂₋₆alkynyl;
R² is selected from the group consisting of hydrogen, C₁₋₆alkyl, C₂₋₆alkenyl, C₂₋₆alkynyl, and C₁₋₆acyl;
R³ is selected from the group consisting of hydrogen, C₁₋₆alkyl, C₂₋₆alkenyl, and C₂₋₆alkynyl;
R⁴, independently for each occurrence, is selected from the group consisting of halo, C₁₋₆alkyl, C₂₋₆alkenyl, C₂₋₆alkynyl, C₁₋₆alkoxy, —C(O)—O—R⁵, and —C(O)—N(R⁵)₂;
R⁵, independently for each occurrence, is selected from the group consisting of hydrogen, C₁₋₆alkyl, C₂₋₆alkenyl, and C₂₋₆alkynyl;
wherein any of R¹, R², R³, and R⁴, independently and independently for each occurrence, is optionally substituted with 1-3 substituents selected from the group consisting of C₁₋₁₀alkyl, C₁₋₁₀haloalkyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl; and wherein heterocyclic or heteroaromatic rings, independently for each occurrence, comprise 1-4 heteroatoms selected from N, O, and S;
n is 0-2;
m is 0-3; and
wherein the composition is edible and capable of reducing bitter taste of a bitter tastant;
(c) Formula (VIII): 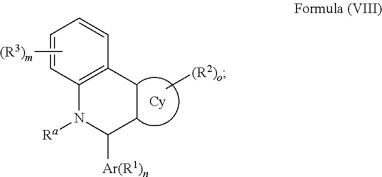
or a comestibly or biologically acceptable salt or derivative thereof,
wherein, as valence and stability permit:
R¹, independently for each occurrence, is selected from the group consisting of halo, hydroxyl, C₁₋₆alkyl, C₁₋₆haloalkyl, C₂₋₆alkenyl, C₂₋₆alkynyl, C₁₋₆alkoxy, and C₁₋₆acyloxy;
R², independently for each occurrence, is C₁₋₆alkyl;
R³, independently for each occurrence, is selected from the group consisting of halo, C₁₋₆alkyl, C₂₋₆alkenyl, C₂₋₆alkynyl, C(O)—O—R⁴, and C(O)—N(R⁴)₂;
R⁴, independently for each occurrence, is selected from the group consisting of hydrogen, C₁₋₆alkyl, C₂₋₆alkenyl, and C₂₋₆alkynyl;
R^{a} is selected from the group consisting of hydrogen, C₁₋₆alkyl, C₂₋₆alkenyl, and C₂₋₆alkynyl;
Ar is selected from the group consisting of C₆₋₁₀aryl and C₃₋₉heteroaryl;
Cy is a 5 to 7-membered carbocyclic or heterocyclic ring, optionally including one or two carbon-carbon or carbon-nitrogen double bonds in the ring;
wherein any of R¹, R², R³, and R^{a}, independently and independently for each occurrence, is optionally substituted with 1-3 substituents selected from the group consisting of C₁₋₁₀alkyl, C₁₋₁₀haloalkyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl; and wherein heterocyclic or heteroaromatic rings, independently for each occurrence, comprise 1-4 heteroatoms selected from N, O, and S;
m is 1-3;
n is 0-3;
o is 0-3; and
wherein the composition is edible and capable of reducing bitter taste of a bitter tastant;
(d) Formula (XI): 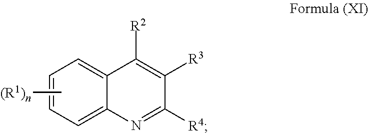
or a comestibly or biologically acceptable salt or derivative thereof,
wherein, as valence and stability permit:
R¹, independently for each occurrence, is selected from the group consisting of halo, hydroxyl, C₁₋₆alkyl, C₁₋₆haloalkyl, C₂₋₆alkenyl, C₂₋₆alkynyl, and C₁₋₆alkoxy;
R² is selected from the group consisting of hydrogen, halo, hydroxyl, C₁₋₆alkyl, C₁₋₆haloalkyl, Het-C₁₋₆alkyl, C₂₋₆alkenyl, C₂₋₆alkynyl, and C₁₋₆alkoxy;
R³ is selected from the group consisting of hydrogen, halo, hydroxyl, C₁₋₆alkyl, C₁₋₆haloalkyl, Het-C₁₋₆alkyl, C₂₋₆alkenyl, C₂₋₆alkynyl, and C₁₋₆alkoxy;
R⁴ is selected from the group consisting of hydrogen, halo, hydroxyl, C₁₋₆alkyl, C₁₋₆haloalkyl, Het-C₁₋₆alkyl, C₂₋₆alkenyl, C₂₋₆alkynyl, and C₁₋₆alkoxy;
or R³ and R⁴ together with the atoms to which they are attached form a 5 to 6-membered aryl ring optionally substituted with 1 to 4 groups selected from the group consisting of halo, hydroxyl, C₁₋₆alkyl, C₁₋₆haloalkyl, C₂₋₆alkenyl, C₂₋₆alkynyl, C₁₋₆alkoxy, and Het;
Het is a C₂₋₆heterocyclyl including 1-3 heteroatoms in the ring selected from oxygen, sulfur, and nitrogen and is optionally substituted with one or more groups selected from the group consisting of halo, hydroxyl, C₁₋₆alkyl, C₁₋₆haloalkyl, C₂₋₆alkenyl, C₂₋₆alkynyl, C₁₋₆alkoxy, and C₆₋₁₀aryl optionally substituted with C₁₋₆alkyl;
wherein any of R¹, R², R³, R⁴, and Het, independently and independently for each occurrence, is optionally further substituted with 1-3 substituents selected from the group consisting of C₁₋₁₀alkyl, C₁₋₁₀haloalkyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, phenyloxy, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, C₆₋₁₀aryl, C₁₋₆alkyl-C₆₋₁₀aryl, C₆₋₁₀aryl-C₁₋₆alkyl, C₁₋₅heteroaryl, and C₁₋₅heteroaryl-C₁₋₆alkyl; and wherein heterocyclic or heteroaromatic rings, independently for each occurrence, comprise 1-4 heteroatoms selected from N, O, and S; and
n is 0-4; and
wherein the composition is edible and capable of reducing bitter taste of a bitter tastant; and
(e) Formula (XIV): 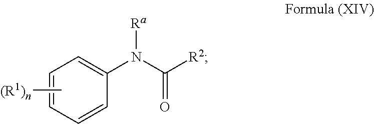
or a comestibly or biologically acceptable salt or derivative thereof,
wherein, as valence and stability permit:
R¹, independently for each occurrence, is selected from the group consisting of halo, hydroxyl, C₁₋₆alkyl, C₁₋₆haloalkyl, C₂₋₆alkenyl, C₂₋₆alkynyl, C₁₋₆alkoxy, and C₁₋₆acyloxy;
R² is selected from the group consisting of C₁₋₆alkyl, C₁₋₆alkoxy-substituted C₁₋₆alkyl, C₆₋₁₀aryloxy-substituted C₁₋₆alkyl, C₂₋₆alkenyl, C₂₋₆alkynyl, C₆₋₁₀aryl-C₁₋₆alkyl, and —((CH₂)ₘX)ₚ—Ar, wherein aryl groups of R² are optionally substituted with one or more halo, hydroxyl, C₁₋₆alkyl, C₁₋₆haloalkyl, C₁₋₆alkoxy, or C₁₋₆acyloxy;
R^{a} is selected from the group consisting of hydrogen, C₁₋₆alkyl, C₂₋₆alkenyl, and C₂₋₆alkynyl;
X is selected from the group consisting of O, NH, and CH₂;
Ar is selected from the group consisting of C₆₋₁₀aryl, C₄₋₉heteroaryl, C₅₋₁₀carbocyclyl, and C₄₋₉heterocyclyl, including fused bicyclic groups, wherein Ar is optionally substituted with one or more halo, hydroxyl, C₁₋₆alkyl, C₁₋₆haloalkyl, C₁₋₆alkoxy, or C₁₋₆acyloxy;
wherein any of R¹, R², and R^{a}, independently and independently for each occurrence, is optionally further substituted with 1-3 substituents selected from the group consisting of C₁₋₁₀alkyl, C₁₋₁₀haloalkyl, halo, hydroxyl, carboxyl, C₁₋₁₀alkoxycarbonyl, C₂₋₁₀alkenyloxycarbonyl, C₂₋₁₀alkynyloxycarbonyl, C₁₋₁₀acyl, C₁₋₁₀acylamino, C₁₋₁₀acyloxy, C₁₋₁₀carbonate, C₁₋₁₀alkoxy, C₆₋₁₀aryloxy, C₆₋₁₀arylamino, phosphoryl, phosphate, phosphonate, phosphinate, amino, diC₁₋₁₀alkylamino, monoC₁₋₁₀alkylamino, C₁₋₁₃amido, C₁₋₁₀imino, C₁₋₁₀carbamate, C₁₋₁₀urea, cyano, nitro, azido, sulfhydryl, C₁₋₁₀alkylthio, sulfate, sulfonate, sulfamoyl, sulfonamido, sulfonyl, C₃₋₇carbocyclyl, C₃₋₇carbocyclyl-C₁₋₆alkyl, C₁₋₆heterocyclyl, C₁₋₆heterocyclyl-C₁₋₆alkyl, phenyl, phenyl-C₁₋₆alkyl, C₁₋₅heteroaryl, and C₁₋₅heteroaryl-C₁₋₆alkyl; and wherein heterocyclic or heteroaromatic rings, independently for each occurrence, comprise 1-4 heteroatoms selected from N, O, and S;
m is 1-3;
n is 0-3;
p is 0 or 1; and
wherein the composition is edible and capable of reducing bitter taste of a bitter tastant.

2. The composition according to claim 1, wherein said compound according to Formula (I) is a compound selected from:
(a) a compound of Formula (IIa): 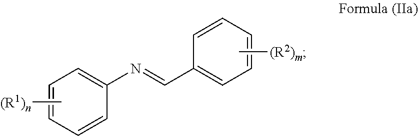
or a comestibly or biologically acceptable salt or derivative thereof,
wherein, as valence and stability permit, R¹, R², m, and n are as defined for the compound of Formula (I) in claim 1; or
(b) a compound of Formula (IIb): 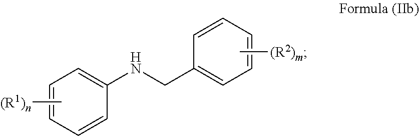
or a comestibly or biologically acceptable salt or derivative thereof,
wherein, as valence and stability permit, R¹, R², m, and n are as defined for the compound of Formula (I) in claim 1.

3. The composition according to claim 1, wherein said compound according to Formula (I) is a compound selected from:
(a) a compound of Formula (IIIb): 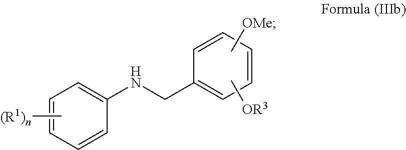
or a comestibly or biologically acceptable salt or derivative thereof,
wherein, as valence and stability permit:
R¹ and n are as defined for the compound of Formula (I) in claim 1; and
R³ is selected from the group consisting of methyl and ethyl;
(b) a compound of Formula (IIIb′): 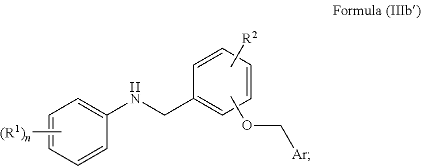
or a comestibly or biologically acceptable salt or derivative thereof,
wherein, as valence and stability permit:
R¹, R², and n are as defined for the compound of Formula (I) in claim 1; and
Ar is C₆₋₁₀aryl optionally substituted with halo, hydroxyl, C₁₋₆alkyl, C₁₋₆alkoxy, or C₁₋₆acyloxy; or
(c) a compound of Formula (IIIb″): 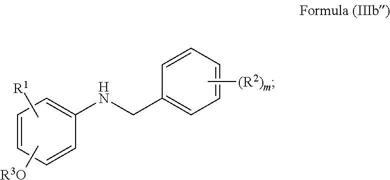
or a comestibly or biologically acceptable salt or derivative thereof,
wherein, as valence and stability permit:
R¹, R², and m are as defined for the compound of Formula (I) in claim 1; and
R³ is C₁₋₆alkyl.

4. The composition according to claim 1, wherein said compound according to Formula (IV) is a compound selected from:
(a) a compound of Formula (Va): 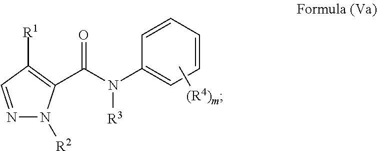
or a comestibly or biologically acceptable salt or derivative thereof,
wherein, as valence and stability permit, R¹, R², R³, R⁴, and m are as defined for the compound of Formula (IV) in claim 1; or
(b) a compound of Formula (Vb): 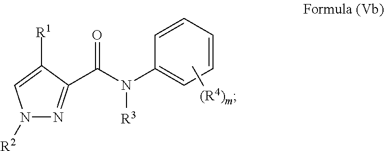
or a comestibly or biologically acceptable salt or derivative thereof,
wherein, as valence and stability permit, R¹, R², R³, R⁴, and m are as defined for the compound of Formula (IV) in claim 1.

5. The composition according to claim 1, wherein said compound according to Formula (IV) is a compound selected from:
(a) a compound of Formula (VIa): 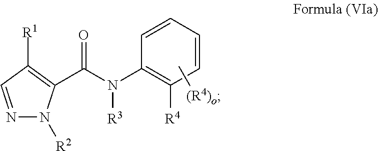
or a comestibly or biologically acceptable salt or derivative thereof,
wherein, as valence and stability permit:
R¹, R², R³, R⁴, are as defined for the compound of Formula (IV) in claim 1; and
o is 0-2; or
(b) a compound of Formula (VIb): 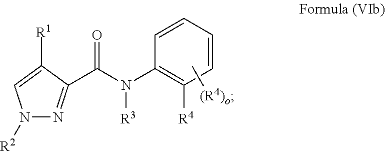
or a comestibly or biologically acceptable salt or derivative thereof,
wherein, as valence and stability permit:
R¹, R², R³, R⁴, are as defined for the compound of Formula (IV) in claim 1; and
o is 0-2.

6. The composition according to claim 1, wherein said compound according to Formula (IV) is a compound selected from:
(a) a compound of Formula (VIIa): 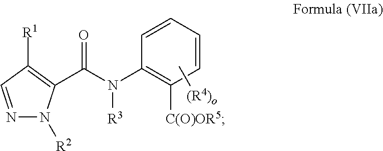
or a comestibly or biologically acceptable salt or derivative thereof,
wherein, as valence and stability permit:
R¹, R², R³, R⁴ and R⁵ are as defined for the compound of Formula (IV) in claim 1; and
o is 0-2; or
(b) a compound of Formula (VIIb): 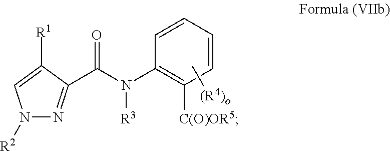
and comestibly or biologically acceptable derivatives thereof,
wherein, as valence and stability permit, R¹, R², R³, R⁴ and R⁵ are as defined for the compound of Formula (IV) in claim 1; and
o is 0-2.

7.-10. (canceled)

11. The composition according to claim 1, wherein said compound according to Formula (XIV) is a compound selected from:
(a) a compound of Formula (XVa): 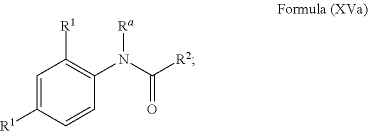
or a comestibly or biologically acceptable salt or derivative thereof,
wherein, as valence and stability permit:
R¹ and R^{a} are as defined for the compound of Formula (XIV) in claim 1; and
R² is C₁₋₆alkyl;
(b) a compound of Formula (XVb): 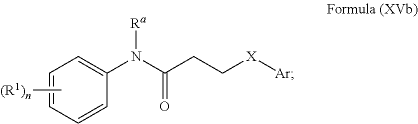
or a comestibly or biologically acceptable salt or derivative thereof,
wherein, as valence and stability permit:
R¹, R^{a}, X, Ar, and n are as defined for the compound of Formula (XIV) in claim 1; or
(c) a compound of Formula (XVc): 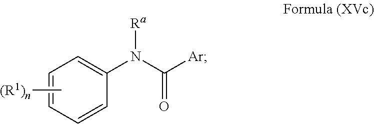
or a comestibly or biologically acceptable salt or derivative thereof, wherein, as valence and stability permit, R¹, R^{a}, Ar, and n are as defined for the compound of Formula (XIV) in claim 1.

12. The composition according to claim 1, wherein the composition comprises a compound selected from the group consisting of Compounds 1-58 or mixtures of compounds 1-58: 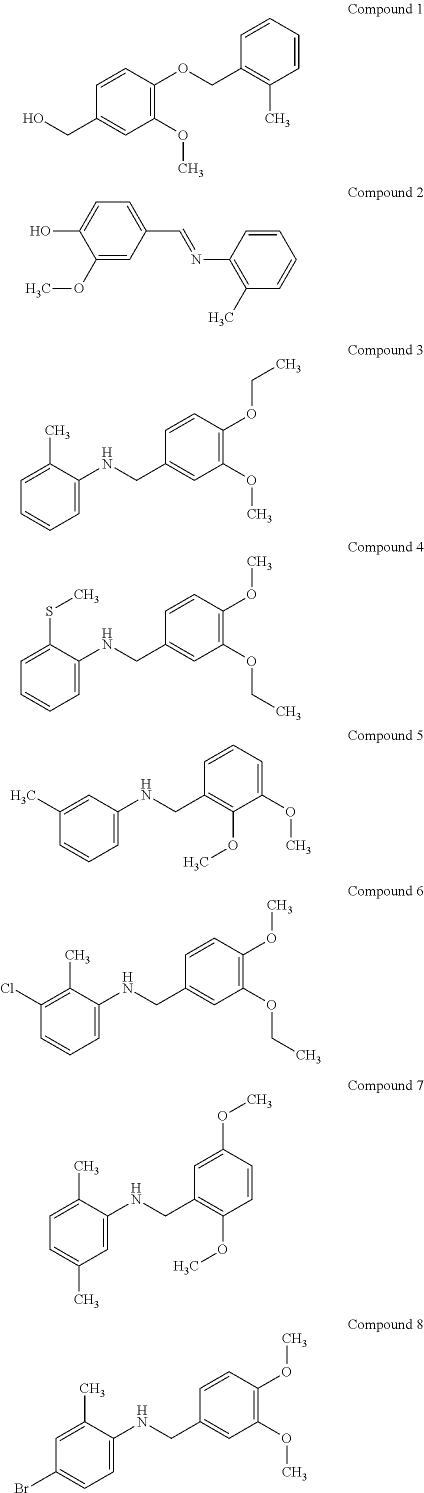 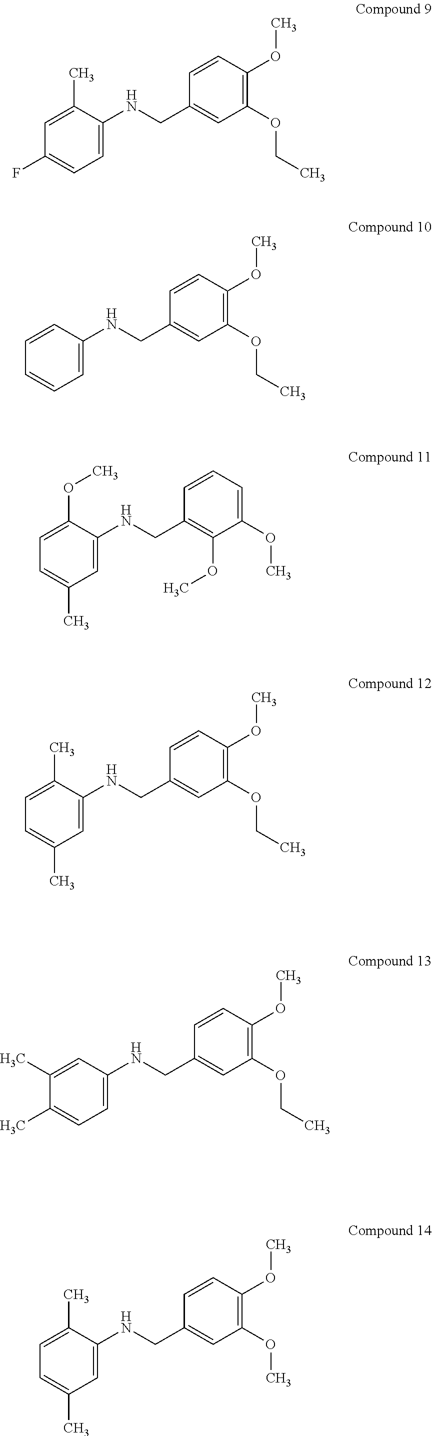 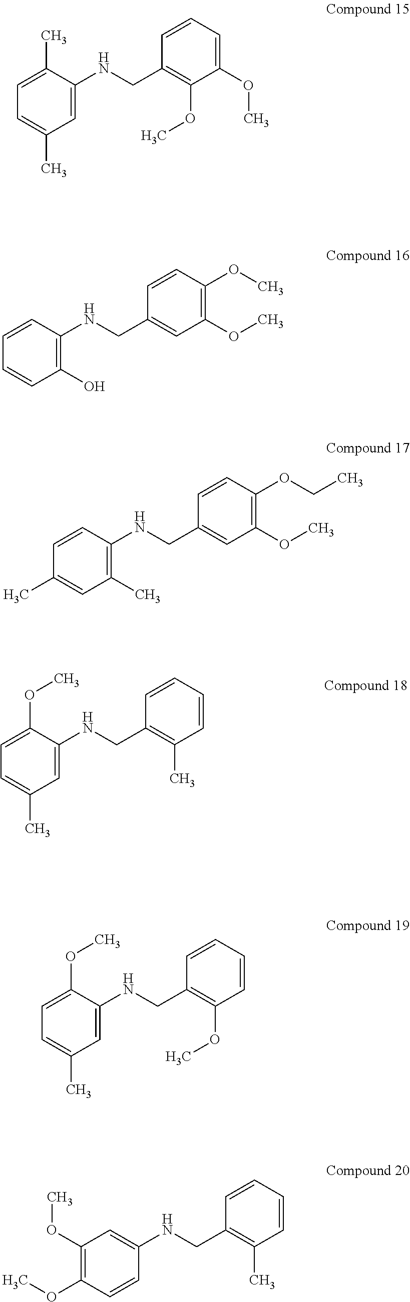 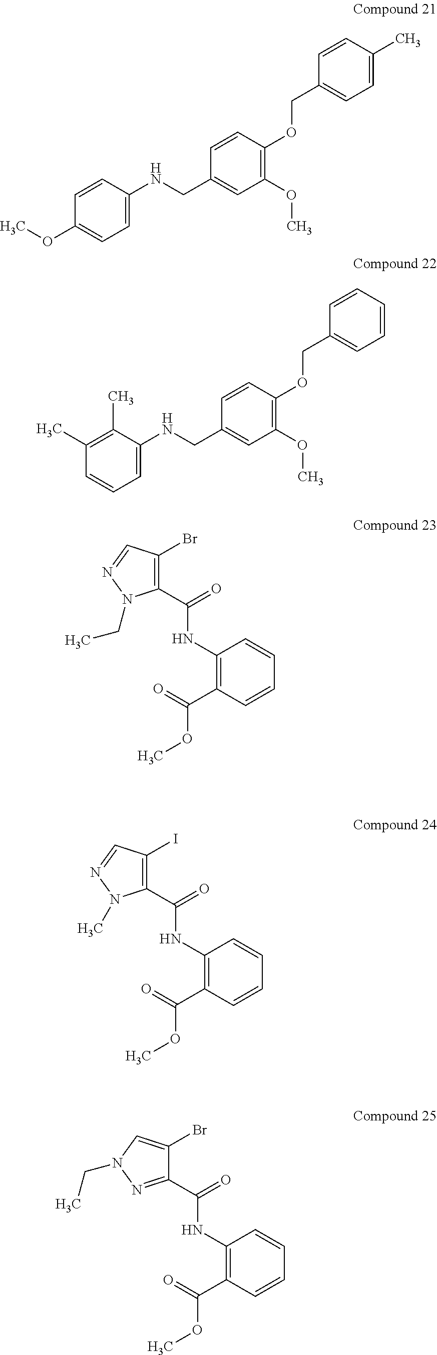 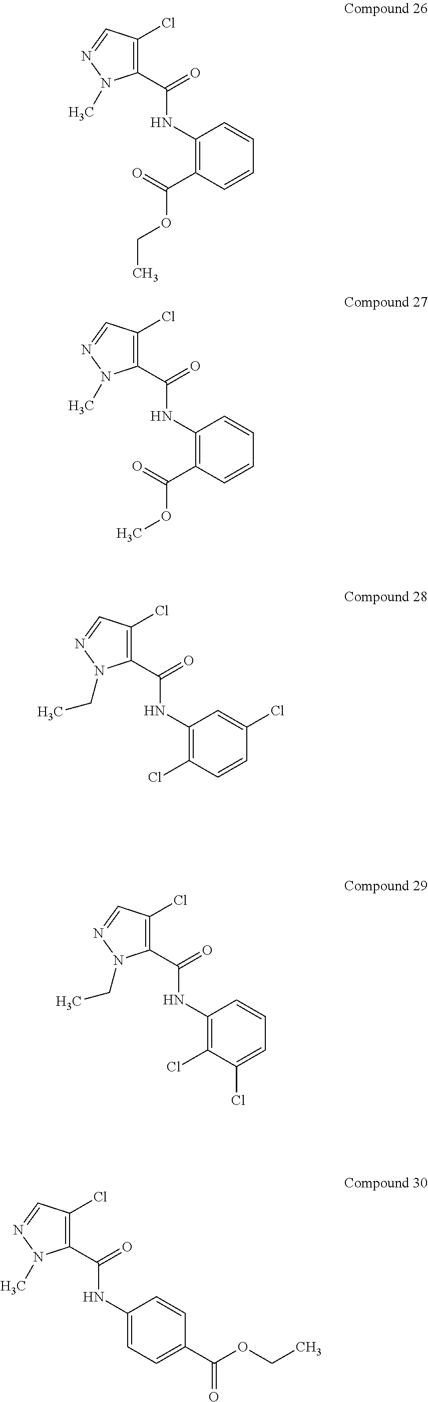 [SMILES: CCOC(=O)c1ccccc1NC(=O)c1c(Cl)cnn1C ; COC(=O)c1ccccc1NC(=O)c1c(Cl)cnn1C ; CCn1ncc(Cl)c1C(=O)Nc1cc(Cl)ccc1Cl ; CCn1ncc(Cl)c1C(=O)Nc1cccc(Cl)c1Cl ; CCOC(=O)c1ccc(NC(=O)c2c(Cl)cnn2C)cc1] 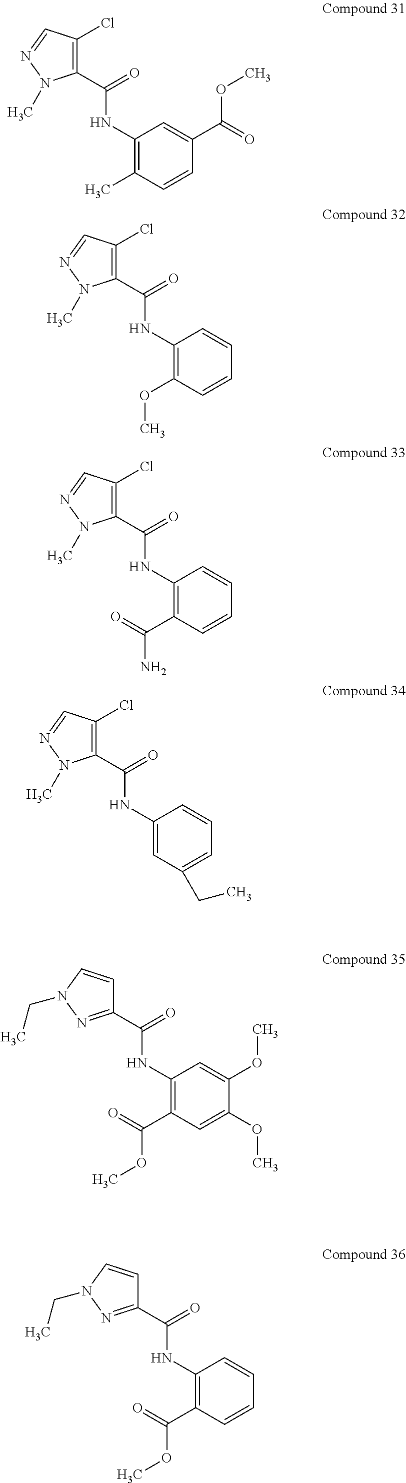 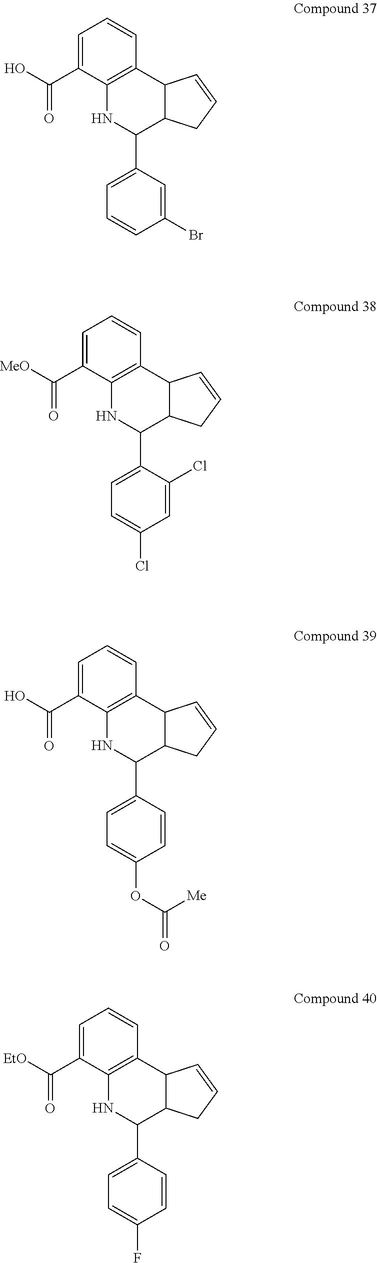 [SMILES: O=C(O)c1cccc2c1NC(c1cccc(Br)c1)C1CC=CC21 ; COC(=O)c1cccc2c1NC(c1ccc(Cl)cc1Cl)C1CC=CC21 ; CC(=O)Oc1ccc(C2Nc3c(C(=O)O)cccc3C3C=CCC32)cc1 ; CCOC(=O)c1cccc2c1NC(c1ccc(F)cc1)C1CC=CC21] 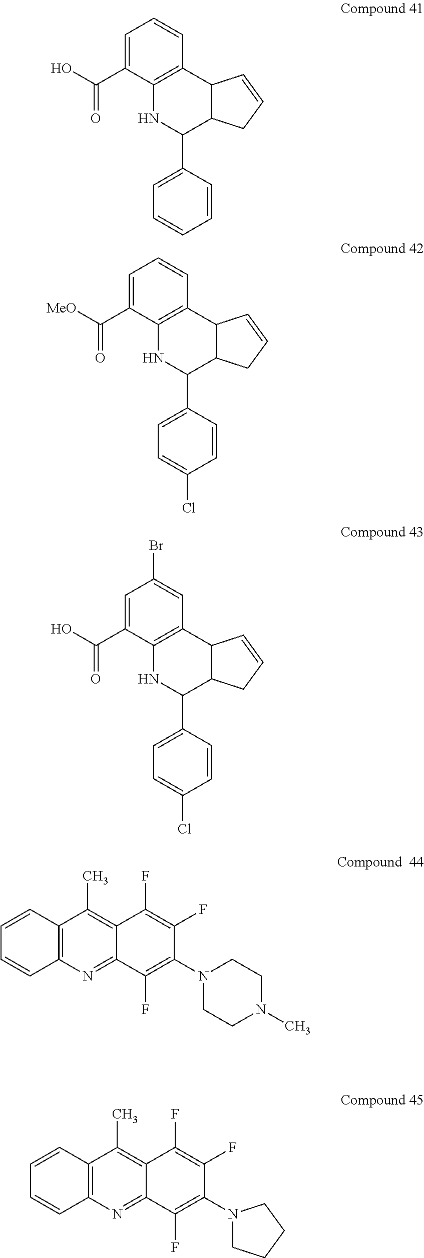 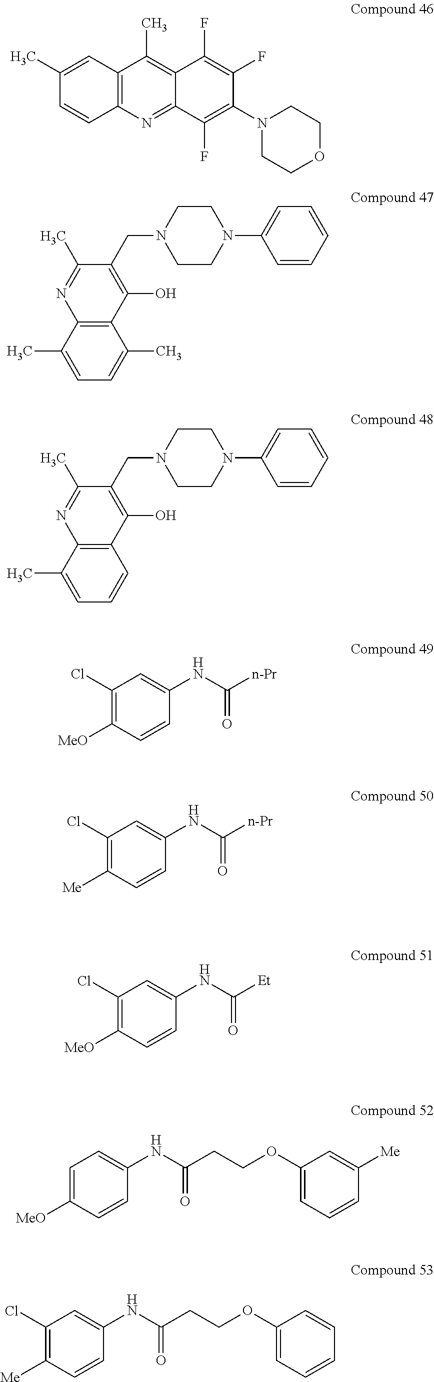 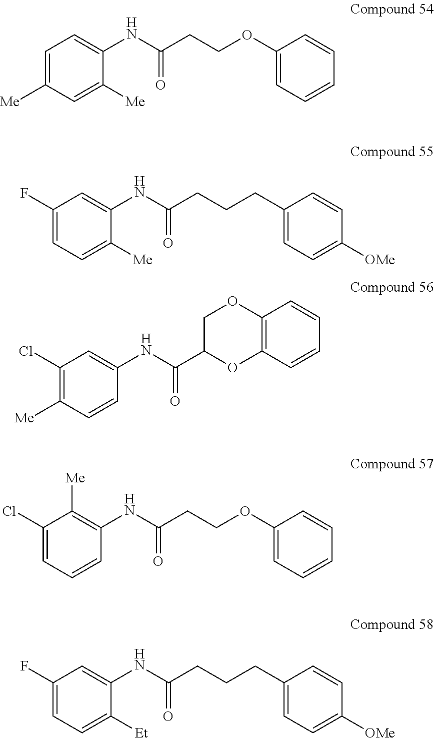 [SMILES: Cc1ccc(NC(=O)CCOc2ccccc2)c(C)c1 ; COc1ccc(CCCC(=O)Nc2cc(F)ccc2C)cc1 ; Cc1ccc(NC(=O)C2COc3ccccc3O2)cc1Cl ; Cc1c(Cl)cccc1NC(=O)CCOc1ccccc1 ; CCc1ccc(F)cc1NC(=O)CCCc1ccc(OC)cc1]
or comestibly or biologically acceptable derivatives thereof.

13.-55. (canceled)

56. The composition of any one of claims 1-6, 11 or 12, further comprising a bitter tastant.

57. The composition of any one of claims 1-6, 11, 12 or 56, wherein the composition further comprises one or more components selected from the group consisting of: NaCl, sodium lactate and sugar.

58. The composition of any one of claims 1-6, 11, 12, 56 or 57, wherein the composition is found in a food product or a consumer product.

59. The composition of any one of claims 1-6, 11 or 12, wherein the composition further comprises a pharmaceutically active ingredient, and optionally, a bitter tastant.

60. The composition of claim 59, wherein the pharmaceutically active ingredient is bitter.

61. The composition of claim 56 or 59, wherein the pharmaceutically active ingredient or bitter tastant is a potassium salt.

62. The composition of any one of claims 56, 58 or 61, wherein the bitter tastant is a potassium salt, and wherein the potassium salt is potassium chloride or potassium lactate.

63. A method of use of the composition of any one of claims 1-6, 11 or 12, wherein the method comprises:
(a) providing a comestibly acceptable carrier; and
(b) adding to the comestibly acceptable carrier, a composition as described in any of claims 1-8 or combinations thereof.

64. The method of claim 63, wherein the method further comprises adding a bitter tastant to the comestibly acceptable carrier.

65. The method of claim 63 or 64 wherein the comestibly acceptable carrier:
i) is inherently bitter; or
ii) is a potassium salt.

66. A method of use of the composition of any one of claims 1-6, 11 or 12, wherein the method comprises use of an effective amount of the composition of any one of claims 1-8 to:
i) reduce or replace one or more sodium salts with one or more potassium salts to reduce the amount of sodium salt in an edible composition; or
ii) reduce or replace the amount of sugar in an edible composition containing a bitter tastant or a potassium salt; or
iii) preserve an edible composition in the presence of potassium lactate; or
iv) reduce or replace the amount of sodium in an edible composition containing a bitter tastant or a potassium salt while preserving the edible composition.

67. The method of claim 66(i) or claim 66(iv), wherein the effective amount of the composition in the edible composition is sufficient to permit reduction or replacement of the amount of sodium salt present in the edible composition by up to 25%, 50%, 75% or 100%.

68. The method of claim 66 or 67, wherein perception of a bitter taste in the oral cavity of a subject is inhibited, reduced or eliminated.

69. The method of claim 68, wherein the composition of any one of claims 1-8 is ingested, in an effective amount, before, together with, or after the ingestion of a bitter tastant, to reduce the bitter taste of the bitter tastant.

70. The method of any of claims 66-69, wherein the edible composition with reduced salt retains a salty flavor.

71. The method of any of claims 66-69, wherein the edible composition with reduced sugar maintains a sweet flavor.

72. The method of any of claims 66-69, wherein the edible composition is preserved by
replacing an amount of sodium lactate used in preparing the edible composition with an amount of potassium lactate, and wherein the edible composition that is preserved has the same shelf life as the edible composition with the original amount of sodium lactate.

73. The method of claim 66 or 68, wherein the amount of sugar in an edible composition is replaced with an amount of Acesulfame K.

74. The method of claim 66 or 68, wherein the amount of sodium chloride in an edible composition is replaced with an amount of potassium chloride.

75. The method of any one of claims 66-69, 73 or 74, wherein the edible composition is selected from the group consisting of a food product, a consumer product, and a pharmaceutical composition.
